(19) United States
(12) Patent Application Publication
(10) Pub. No.: US 2024/0066109 A1
(43) Pub. Date: Feb. 29, 2024

(54) Klebsiella Pneumoniae O-Antigen Glycosylated Proteins and Methods of Making and Uses Thereof

(71) Applicant: VaxNewMo LLC, St. Louis, MO (US)

(72) Inventors: Christian Michael Harding, St. Louis, MO (US); Cory James Knoot, St. Louis, MO (US)

(21) Appl. No.: 18/270,043

(22) PCT Filed: Dec. 29, 2021
(86) PCT No.: PCT/US2021/065533

§ 371 Date: Jun. 28, 2023

Related U.S. Application Data
(60) Provisional application No. 63/132,610, filed on Dec. 31, 2020.

(51) Int. Cl. A61K 39/108; A61K 47/64; A61P 31/04; C07K 14/26

(52) U.S. Cl. CPC A61K 39/0266; A61K 47/646; A61P 31/04; C07K 14/26; A61K 2039/6037

(57) ABSTRACT

Provided herein is a bioconjugate comprising a K. pneumoniae O-antigen covalently linked to a fusion protein comprising a ComP protein or a glycosylation tag fragment. The K. pneumoniae O-antigen bioconjugate of this disclosure can be used as a conjugate vaccine including multivalent conjugate vaccines comprising multiple K. pneumoniae O-antigens.

### CROSS-REFERENCE TO RELATED APPLICATIONS

This application claims the benefit of U.S. Provisional Patent Application No. 62/132,610, filed Dec. 31, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 29, 2021, is named 64100-215516_Seq_List_ST25.txt and is 99,075 bytes in size.

### BACKGROUND

The World Health Organization (WHO) predicts that by 2050, drug resistant infections could be responsible for 10 million deaths globally each year (Resistance, I. C. G. o. A. NO TIME TO WAIT: SECURING THE FUTURE FROM DRUG-RESISTANT INFECTIONS, on the world wide web at www.whoint/docs/default-source/documents/no-time -to-wait-securing-the-future-from-drug-resistant-infections-en.pdf?sfvrsn=5b424d7_6>, 2019). This total would surpass diabetes, heart disease and cancers as the leading cause of all human deaths annually. In addition, the Center for Disease Control and Prevention (CDC) reported that 2.8 million antibiotic-resistant infections occur in the United States each year with 35,000 Americans dying as a result (CDC. ANTIBIOTIC RESISTANCE THREATS IN THE UNITED STATES 2019, on the world wide web at www.cdc.gov/drugresistance/pdf/threats-report/2019-ar-threats-report-508.pdN. While new antibiotics are needed, they will not solve the problem of antibiotic resistance as resistance to next generation antibiotics will likely become commonplace. One of the most frequently encountered antibiotic resistant bacteria causing human infections is Klebsiella pneumoniae (K. pneumoniae). K. pneumoniae is a Gram-negative bacterium causing healthcare- and community-associated infections. Moreover, K. pneumoniae is frequently resistant to multiple classes of antibiotics like third generation cephalosporins and carbapenems. As such, K. pneumoniae is a member of the Extended Spectrum β-Lactamase (ESBL)-producing Enterobacteriaceae and Carbapenem-Resistant Enterobacteriaceae (CRE) groups, which are considered urgent threats by the CDC and the World Health Organization (WHO). The Center for Disease Control and Prevention's 2013 and 2019 Antibiotic Resistance Threats in the U.S. reports estimate that ESBL-producing K. pneumoniae and carbapenem resistant K. pneumoniae are responsible for >157,000 infections causing >7,500 deaths each year (CDC. ANTIBIOTIC RESISTANCE THREATS IN THE UNITED STATES 2019, on the world wide web at www.cdc.gov/drugresistance/pdf/threats -report/2019-ar-threats-report-508.pdf>; CDC. ANTIBIOTIC RESISTANCE THREATS in the United States, 2013, on the world wide web at www.cdc.gov/drugresistance/pdf/ar-threats-2013-508.pdf5). Moreover, K. pneumoniae accounts for 5% of all healthcare-associated infections (HAIs) in the United States each year (Magill, S. S. et al., 2018). In Europe, multidrug resistant K. pneumoniae are responsible for >90,000 infections and >7,000 deaths each year, accounting for 25% of all disability-adjusted life years lost due to MDR infections (Cassini, A. et al., 2019) Globally, K. pneumoniae has become a leading cause of sepsis and infectious neonatal deaths surpassing Streptococcus pneumoniae, Group B Streptococcus and Staphylococcus aureus in many Low-and-Middle Income Countries according to the Child Health and Mortality Prevention Surveillance. Given its global prevalence and frequent antibiotic resistant phenotypes, an efficacious vaccine preventing K. pneumoniae infections would be of immense societal benefit. The most successful anti-bacterial vaccine strategies over the last three decades have been conjugate vaccines. Indeed, the first conjugate vaccines licensed, targeting Haemophilus influenzae type b (Hib), have nearly eradicated invasive Hib disease in infants (Policy, N. I. o. H. O. o. S. CHILDHOOD Hib VACCINES: NEARLY ELIMINATING THE THREAT OF BACTERIAL MENINGITIS, on the world wide web at www.nih.gov/sites/default/files/about -nih/impact/childhood-hib-vaccines-case-study.pdf). Further, multiple pneumococcal conjugate vaccines have been licensed over the last two decades resulting in significant reductions in invasive pneumococcal disease for infant and adult populations (Daniels, C. C., Rogers, P. D. & Shelton, C. M., 2016). In addition, studies have demonstrated a significant reduction in the prevalence of antibiotic resistant S. pneumoniae after the introduction of pneumococcal conjugate vaccines (Hampton, L. M. et al. , 2012; Tomczyk, S. et al., 2016; Cohen, R., Cohen, J. F., Chalumeau, M. & Levy, C., 2017). From an economic standpoint, Prevnar 13 (the current standard of care vaccine for preventing pneumococcal disease) has been Pfizer's best-selling product over the last five years with sales approaching $30B USD. Collectively, the societal and economic impacts associated with conjugate vaccines continue to incentivize pharmaceutical companies to invest in and develop next generation conjugate vaccines against existing and emerging bacterial threats. Compositionally, conjugate vaccines consist of two macromolecules, a bacterial polysaccharide covalently attached to an immunogenic carrier protein. The covalent linkage of the polysaccharide to the carrier protein is essential as polysaccharide only vaccines are poor immunogens and do not elicit booster responses, IgM to IgG class switching, or memory responses (Avci, F. Y., Li, X., Tsuji, M. & Kasper, D. L., 2011; Rappuoli, R., De Gregorio, E. & Costantino, P., 2019). It is widely known and accepted that manufacturing conjugate vaccines is extremely complex requiring hundreds of release controls. Further, extensive technical know-how is essential to ensure that purified polysaccharides are correctly linked to carrier proteins and that this process does not alter or destroy the polysaccharide epitopes important for immunogenicity (Frasch, C. E., 2009). Case in point, the world's most complicated licensed drug to manufacture is Pfizer's pneumococcal conjugate vaccine, Prevnar 13, which according to its own manufacturing reports takes 2.5 years to make one dose from start to fill (Pfizer. Pfizer 2015 Annual Report: Manufacturing and Supply Chain. 5 on the world wide web at Pfizer.com, 2015).

Thus, there remains a need to develop new conjugate vaccines against existing and emerging bacterial threats that are simpler and less expensive to produce.

### SUMMARY

Provided for herein is a bioconjugate comprising a K. pneumoniae O-antigen covalently linked to a fusion protein, wherein the fusion protein comprises a ComP protein or a glycosylation tag fragment thereof. In certain embodiments, the O-antigen has not been derivatized by: i) being subject to oxidation/reduction procedures; ii) activated with 1-Cyano-4-Dimethylaminopyridine Tetrafluoroborate (CDAP); iii) the addition of primary amines; and/or iv) the addition of diamine spacer molecules. In certain embodiments the O-antigen is underivatized. In certain embodiments, the O-antigen is a native O-antigen. In certain embodiments, the K. pneumoniae O-antigen is selected from the group consisting of O1, O2, O3, O4, O5 O7, O8 and O12; the K. pneumoniae O-antigen is selected from the group consisting of O1v1, O1v2, O2v1, O2v2, O3, O3a, and O3b; and/or the K. pneumoniae O-antigen is selected from the group consisting of O1afg, O2afg, O2aeh, and O2ac.

In certain embodiments, the bioconjugate is immunogenic. In certain embodiments, the bioconjugate is a conjugate vaccine. Thus, certain embodiments provide for a bioconjugate for use as a conjugate vaccine.

Provided for herein is a conjugate vaccine composition comprising the bioconjugate of this invention. In certain embodiments, the conjugate vaccine composition is a multivalent vaccine comprising at least two, three, four, five, six, or seven of the bioconjugates, each comprising a different K. pneumoniae O-antigen. In certain embodiments, the conjugate vaccine composition comprises: (i) a bioconjugate comprising an O1v1 antigen; (ii) a bioconjugate comprising an O1v2 antigen; (iii) a bioconjugate comprising an O2v1 antigen; (iv) a bioconjugate comprising an O2v2 antigen; (v) a bioconjugate comprising an O3 antigen; (vi) a bioconjugate comprising an O3b antigen; and (vii) a bioconjugate comprising an O5 antigen.

Provided for herein is a fusion protein comprising ComP or a glycosylation tag fragment thereof and a Pseudomonas aeruginosa exotoxin A (EPA) carrier protein, a CRM₁₉₇ carrier protein, a tetanus toxin C fragment carrier protein, or a K. pneumoniae MrkA carrier protein. In certain embodiments, the fusion protein is covalently linked to a K. pneumoniae O-antigen as described herein.

Provided for herein is a method of producing a bioconjugate, the method comprising covalently linking a K. pneumoniae O-antigen to a fusion protein with a Pg1S oligosaccharyltransferase (OTase), wherein the fusion protein comprises a ComP protein or a glycosylation tag fragment thereof. In certain embodiments, the ComP protein or glycosylation tag fragment thereof is linked to a heterologous carrier protein.

Provided for herein is a method of inducing a host immune response against K. pneumoniae, the method comprising administering to a subject in need of the immune response an effective amount of the conjugate vaccine composition of this disclosure.

Provided for herein is a method of preventing or treating a K. pneumoniae infection in a subject comprising administering to a subject in need thereof the bioconjugate of this disclosure.

Provided for herein is the use of the bioconjugate, fusion protein, and/or or the conjugate vaccine composition of this disclosure to induce a host immune response against K. pneumoniae, prevent a K. pneumoniae infection, and/or treat a K. pneumoniae infection.

Provided for herein is a method of producing a conjugate vaccine against K. pneumoniae infection, the method comprising: (a) isolating the bioconjugate of this disclosure; and (b) combining the isolated bioconjugate with an adjuvant.

### BRIEF DESCRIPTION OF THE DRAWINGS

FIG. 1. FIG. 1 shows the carbohydrate structures of the repeat units for the O1, O2a, O2aeh, O2afg, O2ac, O3, O4, O5, O7and O12 O-antigens from K. pneumoniae according to Clarke et al., 2018 (Clarke, B. R. et al., 2018).

FIG. 2. FIG. 2 shows the subtype designations for the major O-antigen serogroups of K. pneumoniae.

FIG. 3. FIG. 3 shows the O-serotype distributions for K. pneumoniae isolates that were typed using the Kaptive program (Wick, R. R., Heinz, E., Holt, K. E. & Wyres, K. L., 2018) from three studies (Choi, M. et al., 2020; Artyszuk, D. et al., 2020; Wyres, K. L. et al., 2020).

FIG. 4. FIG. 4A shows schematic of an exemplary modified EPA carrier protein used in this study. FIG. 4B shows the FASTA sequence of an exemplary modified EPA carrier protein used in this study (SEQ ID NO: 117).

FIG. 5. FIG. 5 shows Coomassie stained SDS-PAGE analysis of the purified, modified EPA carrier protein glycosylated with seven different K. pneumoniae O-antigens (O1v1, O1v2, O2v1, O2v2, O3, O3b, O5).

FIG. 6. FIG. 6A shows western blot analysis of the purified, modified EPA carrier proteins glycosylated with seven different K. pneumoniae O-antigens (O1v1, O1v2, O2v1, O2v2, O3, O3b, O5) probed with an anti-His antibody. FIG. 6B shows western blot analysis of the purified, modified EPA carrier protein glycosylated with seven different K. pneumoniae O-antigens (O1v1, O1v2, O2v1, O2v2, O3, O3b, O5) probed with an anti-D-galactan III (afg antigen) antibody. FIG. 6C shows the merged images of the FIG. 6A and FIG. 6B western blots. Co-localization of the D-galactan-III signal with the anti-his signal is only observed in modified EPA carrier proteins glycosylated with D-galactan III epitopes (O1v2 and O2v2 ).

FIG. 7. FIG. 7A shows western blot analysis of the purified, modified EPA carrier proteins glycosylated with either the O1v1 antigen or the O1v2 antigen probed with an anti-His antibody. FIG. 7B shows western blot analysis of the purified, modified EPA carrier protein glycosylated with either the Olvl antigen or the O1v2 antigen probed with an anti-D-galactan II antibody. FIG. 7C shows the merged images of the FIG. 7A and FIG. 7B western blots. Co-localization of the D-galactan-II signal with the anti-his signal is observed in modified EPA carrier proteins glycosylated with D-galactan II epitopes (O1v1 and O1v2).

FIG. 8. FIG. 8A shows western blot analysis of the purified, modified EPA carrier proteins glycosylated with either the O2v1 antigen or the O2v2 antigen probed with an anti-His antibody. FIG. 8B shows western blot analysis of the purified, modified EPA carrier protein glycosylated with either the O2v1 antigen or the O2v2 antigen probed with an anti-D-galactan I antibody. FIG. 8C shows the merged images of the FIG. 8A and FIG. 8B western blots. Co-localization of the D-galactan-I signal with the anti-his signal is observed in modified EPA carrier proteins glycosylated with D-galactan I epitopes (O2v1 and O2v2).

FIG. 9. FIG. 9A shows western blot analysis of the purified, modified EPA carrier proteins glycosylated with either the O3 antigen, the O3b antigen or the O5 antigen probed with an anti-His antibody. FIG. 9A shows western blot analysis of the purified, modified EPA carrier protein glycosylated with either the O3 antigen, the O3b antigen or the O5 antigen probed with an antiserum specific to the E. coli O9 antigen, which is identical in structure to the K. pneumoniae O3 antigen (Vinogradov, E. et al., 2002). Therefore, the E. coli O9 antisera can be used as a surrogate antibody for assessing the K. pneumoniae O3 serogroups (O3, O3a, O3b). FIG. 9C shows the merged images of the FIG. 9A and FIG. 9B western blots. Co-localization of the O3 signal with the anti-His signal is observed in modified EPA carrier proteins glycosylated with O3 serogroups (O3 and O3b) and not O5.

FIG. 10. FIG. 10A shows western blot analysis of the purified, modified EPA carrier proteins glycosylated with either the O3 antigen, the O3b antigen or the O5 antigen probed with an anti-His antibody. FIG. 10A shows western blot analysis of the purified, modified EPA carrier protein glycosylated with either the O3 antigen, the O3b antigen or the O5 antigen probed with an antiserum specific to the E. coli O8 antigen, which is identical in structure to the K. pneumoniae O5 antigen (Vinogradov, E. et al., 2002). Therefore, the E. coli O8 antisera can be used as a surrogate antibody for assessing the K. pneumoniae O5 serotype. FIG. 10C shows the merged images of the FIG. 10A and FIG. 10B western blots. Co-localization of the O5 signal with the anti-His signal is observed in modified EPA carrier proteins glycosylated with the O5 antigen and not the O3 serogroups.

FIG. 11. FIG. 11A shows the intact mass spectra of the purified, modified EPA carrier protein glycosylated with the O1v1 antigen of K. pneumoniae. FIG. 11B panels show zoomed in views of the intact mass spectra from FIG. 11A. The non-glycosylated modified EPA carrier protein has a theoretical molecular weight 79,526.15 Daltons. Each ion peak is separated by approximately 324 Daltons (or atomic mass units), which is the approximate molecular weight of the O1v1 antigen.

FIG. 12. FIG. 12A shows the intact mass spectra of the purified, modified EPA carrier protein glycosylated with the O2v1 antigen of K. pneumoniae. FIG. 12B shows a zoomed in view of the intact mass spec spectra from FIG. 12A. The non-glycosylated modified EPA carrier protein has a theoretical molecular weight 79,526.15 Daltons. Each ion peak is separated by approximately 324 Daltons (or atomic mass units), which is the approximate molecular weight of the O2v1 antigen.

FIG. 13. FIG. 13A shows the intact mass spectra of the purified, modified EPA carrier protein glycosylated with the O2v2 antigen of K. pneumoniae. FIG. 13B shows a zoomed in view of the intact mass spec spectra from FIG. 13A. The non-glycosylated modified EPA carrier protein has a theoretical molecular weight 79,526.15 Daltons. Each ion peak is separated by approximately 486 Daltons (or atomic mass units), which is the approximate molecular weight of the O2v2 antigen.

FIG. 14. FIG. 14A shows the intact mass spectra of the purified, modified EPA carrier protein glycosylated with the O3b antigen of K. pneumoniae. FIG. 14B and FIG. 14c shows a zoomed in view of the intact mass spec spectra from FIG. 14A. The non-glycosylated modified EPA carrier protein has a theoretical molecular weight 79,526.15 Daltons. Each ion peak is separated by approximately 486 Daltons (or atomic mass units), which is the approximate molecular weight of the O3b antigen.

FIG. 15. FIG. 15A shows schematic of an exemplary modified MrkA carrier protein used in this study. FIG. 15B shows the FASTA sequence of an exemplary modified EPA carrier protein used in this study containing the ComP₁₁₀₂₆₄₄₂₈ fragment (SEQ ID NO: 119).

FIG. 16. FIG. 16A shows schematic of an exemplary modified MrkA carrier protein used in this study. FIG. 16B shows the FASTA sequence of an exemplary modified EPA carrier protein used in this study containing the C1 fragment of COMP₁₁₀₂₆₄ fragment at N-terminus of MrkA (SEQ ID NO: 119).

FIG. 17. FIG. 17A shows schematic of an exemplary modified MrkA carrier protein used in this study. FIG. 17B shows the FASTA sequence of an exemplary modified EPA carrier protein used in this study containing the C1 fragment of ComP₁₁₀₂₆₄ fragment at C-terminus of MrkA (SEQ ID NO: 121).

FIG. 18. FIG. 18 shows western blot analysis of periplasmic extracts of E. coli HSTO8 cells expressing a modified MrkA carrier protein (MrkA-ComP₁₁₀₂₆₄Δ28, C1-MrkA and MrkA-C1) at three different temperatures. Western blots were probed with anti-His antisera.

FIG. 19. FIG. 19 shows western blot analysis periplasmic extracts of CLM24 E. coli cells expressing the modified MrkA-C1 carrier protein, PglS_{ADP1}, and one of seven different K. pneumoniae O-antigen expressing plasmids. Western blots were probed with anti-His antisera.

FIG. 20. FIG. 20A shows western blot analysis of the purified, modified MrkA-C1 carrier proteins glycosylated with either the O2v1 antigen or the O2v2 antigen probed with an anti-His antibody. FIG. 20B shows western blot analysis of the purified, modified MrkA-C1 carrier protein glycosylated with either the O2v1 antigen or the O2v2 antigen probed with an anti-D-galactan I antibody. FIG. 20B shows western blot analysis of the purified, modified MrkA-C1 carrier protein glycosylated with either the O2v1 antigen or the O2v2 antigen probed with an anti-D-galactan III antibody.

FIG. 21. FIG. 21A shows the intact mass spectra of the purified, modified MrkA-C1 carrier protein glycosylated with the O2v2 antigen of K. pneumoniae. FIG. 21B shows a zoomed in view of the intact mass spec spectra from FIG. 20A. Each ion peak is separated by approximately 486 Daltons (or atomic mass units), which is the approximate molecular weight of the O2v2 antigen. FIG. 21C shows a second zoomed in view of higher atomic mass units of the intact mass spec spectra from FIG. 21A. Each ion peak is separated by approximately 486 Daltons (or atomic mass units), which is the approximate molecular weight of the O2v2 antigen. FIG. 21C.

FIG. 22. FIG. 22 lists ComP ortholog amino acid sequences. The site of predicted glycosylation is bolded, flanked by a predicted disulfide bond (underlined) linking the predicted alpha beta loop to the beta strand region.

FIG. 23. FIG. 23 shows amino acid sequences of representative ComPΔ28₁₁₀₂₆₄ fusion proteins. FIG. 24 discloses “AAA” as SEQ ID NO: 24, “GGGS” as SEQ ID NO: 23 and “hexa-histidine” as SEQ ID NO: 114.

FIG. 24. FIG. 24 lists ComP Δ428 ortholog amino acid sequences in which the amino acids corresponding to the 28 N-terminal amino acids of SEQ ID NO: 1 (ComP_{ADP1}: AAC45886.1) have been removed. The site of predicted glycosylation is bolded, flanked by a predicted disulfide bond (underlined) linking the predicted alpha beta loop to the beta strand region.

FIG. 25. FIG. 25 shows an alignment of a region ComP sequences including the serine (S) residue (boxed) corresponding to the serine residue at position 84 of SEQ ID NO: 1 (ComP_{ADP1}: AAC45886.1).

### DETAILED DESCRIPTION

### Definitions

It is to be noted that the term “a” or “an” entity refers to one or more of that entity; for example, “a polysaccharide,” is understood to represent one or more polysaccharides. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising” or “comprises” otherwise analogous aspects described in terms of “consisting of,” “consists of,” “consisting essentially of,” and/or “consists essentially of,” and the like are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, unless otherwise stated, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology, but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are recombinant polypeptides that have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Disclosed herein are certain binding molecules, or antigen-binding fragments, variants, or derivatives thereof Unless specifically referring to full-sized antibodies such as naturally-occurring antibodies, the term “binding molecule” encompasses full-sized antibodies as well as antigen-binding fragments, variants, analogs, or derivatives of such antibodies, e.g., naturally-occurring antibody or immunoglobulin molecules or engineered antibody molecules or fragments that bind antigen in a manner similar to antibody molecules.

As used herein, the term “binding molecule” refers in its broadest sense to a molecule that specifically binds an antigenic determinant. As described further herein, a binding molecule can comprise one of more “binding domains.” As used herein, a “binding domain” is a two- or three-dimensional polypeptide structure that can specifically bind a given antigenic determinant, or epitope. A non-limiting example of a binding molecule is an antibody or fragment thereof that comprises a binding domain that specifically binds an antigenic determinant or epitope. Another example of a binding molecule is a bispecific antibody comprising a first binding domain binding to a first epitope, and a second binding domain binding to a second epitope.

The terms “antibody” and “immunoglobulin” can be used interchangeably herein. An antibody (or a fragment, variant, or derivative thereof as disclosed herein comprises at least the variable domain of a heavy chain and at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

The term “bispecific antibody” as used herein refers to an antibody that has binding sites for two different antigens within a single antibody molecule. It will be appreciated that other molecules in addition to the canonical antibody structure can be constructed with two binding specificities. It will further be appreciated that antigen binding by bispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide subunit contained in a vector is considered isolated as disclosed herein. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that might be at any time, determined or interpreted by a judge or an administrative or judicial body to be, “naturally-occurring.”

In certain embodiments, the polynucleotide or nucleic acid is DNA. In other embodiments, a polynucleotide can be RNA.

A “vector” is nucleic acid molecule as introduced into a host cell, thereby producing a transformed host cell. A vector can include nucleic acid sequences that permit it to replicate in a host cell, such as an origin of replication. A vector can also include one or more selectable marker gene and other genetic elements known in the art.

A “transformed” cell, or a “host” cell, is a cell into which a nucleic acid molecule has been introduced by molecular biology techniques. As used herein, the term transformation encompasses those techniques by which a nucleic acid molecule can be introduced into such a cell, including transfection with viral vectors, transformation with plasmid vectors, and introduction of naked DNA by electroporation, lipofection, and particle gun acceleration. A transformed cell or a host cell can be a bacterial cell or a eukaryotic cell.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein the terms “treat,” “treatment,” or “treatment of” (e.g., in the phrase “treating a subject”) refers to reducing the potential for disease pathology, reducing the occurrence of disease symptoms, e.g., to an extent that the subject has a longer survival rate or reduced discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals, including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective, and that contains no additional components that are unacceptably toxic to a subject to which the composition would be administered. Such composition can be sterile.

An “effective amount” of an antibody as disclosed herein is an amount sufficient to carry out a specifically stated purpose. An “effective amount” can be determined empirically and in a routine manner, in relation to the stated purpose.

Overview. As an alternative to conventional methods for manufacturing conjugate vaccines, the bioconjugates disclosed herein utilize a platform technology for producing conjugate vaccines enzymatically in a process termed bioconjugation (WO/2019/241671; WO/2020/131236; U.S. application Ser. No. 17/251,994, which are incorporated herein by reference). Bioconjugation can exploit protein glycosylation systems to generate conjugate vaccines in vivo, for example but not limited to using E. coli as a host organism (Feldman, M. F. et al., 2005; Harding, C. M. & Feldman, M. F., 2019). In general, three components are required for bioconjugation: a genetic cluster encoding for the polysaccharide of interest, a carrier protein to be glycosylated, and a conjugating enzyme to transfer the polysaccharide to the carrier protein. Since bioconjugation is completely performed within E. coli, it seamlessly works with existing pharmaceutical infrastructures for large scale microbial fermentation and downstream purification processes. Moreover, because the entire process is accomplished in E. coli, chemical conjugation is no longer needed, which enables bioconjugate vaccines to present the polysaccharide in its fully native conformation with no chemical alterations. Much like a cell line for monoclonal antibody production, once an engineered strain of E. coli has been established for bioconjugate production, it is able to produce an inexhaustible supply of homogenous product. Finally, this system has built-in flexibility. If the carrier protein or polysaccharide needs to be changed, then a simple plasmid swap or chromosomal integration is undertaken, meaning that multiple conjugate combinations can be assembled and tested rapidly. Where conventional techniques for conjugate vaccine manufacturing struggle to shift with changes in seroepidemiology, bioconjugation is optimally positioned to fill the gap with its dynamic and streamlined production.

K. pneumoniae produces two main surface polysaccharide antigens: capsular polysaccharide (CPS) and lipopolysaccharide (LPS) (Follador, R. et al., 2016). CPS is composed of repeating units of carbohydrates; whereas, LPS consists of the lipid A molecule, a core saccharide and an outermost O-antigen polysaccharide (Whitfield, C. & Trent, M. S., 2014). The CPS and O-antigen polysaccharide are classified into distinct groups based on serological reactivities due to differences in chemical and structural compositions. Further, the CPS and O-antigen polysaccharides are the antigens targeted for incorporation into K. pneumoniae conjugate vaccines in development. While more than 100 K. pneumoniae CPS serotypes have been identified either serologically or genetically (Pan, Y. J. et al., 2015), K. pneumoniae has been shown to produce 9 to 10 serogroups of LPS (Choi, M. et al., 2020; Clarke, B. R. et al., 2018), a much more practical number to target for the development of a K. pneumoniae specific conjugate vaccine. Thus, in ceratin embodiments, the present disclosure is drawn to methods of making and uses thereof for K. pneumoniae O-antigen glycosylated proteins.

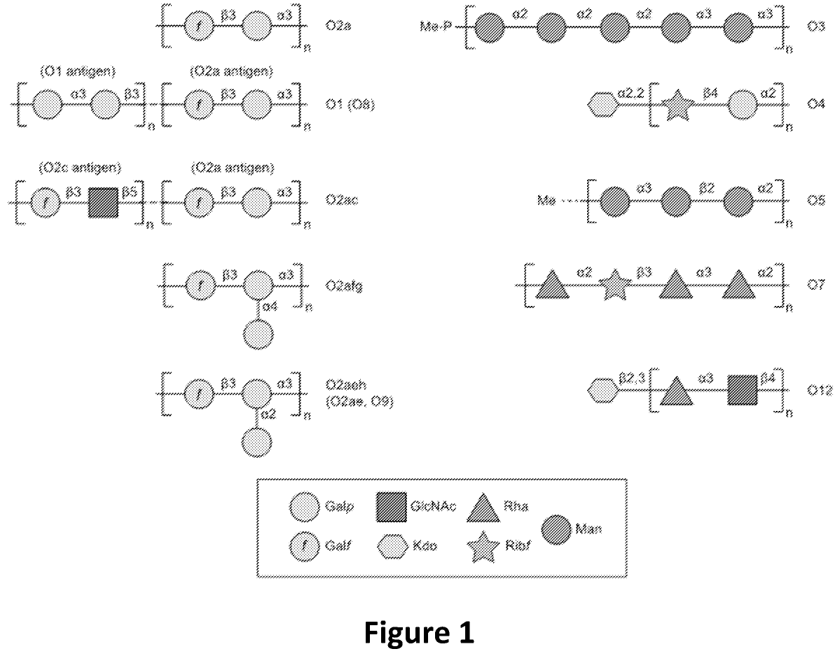

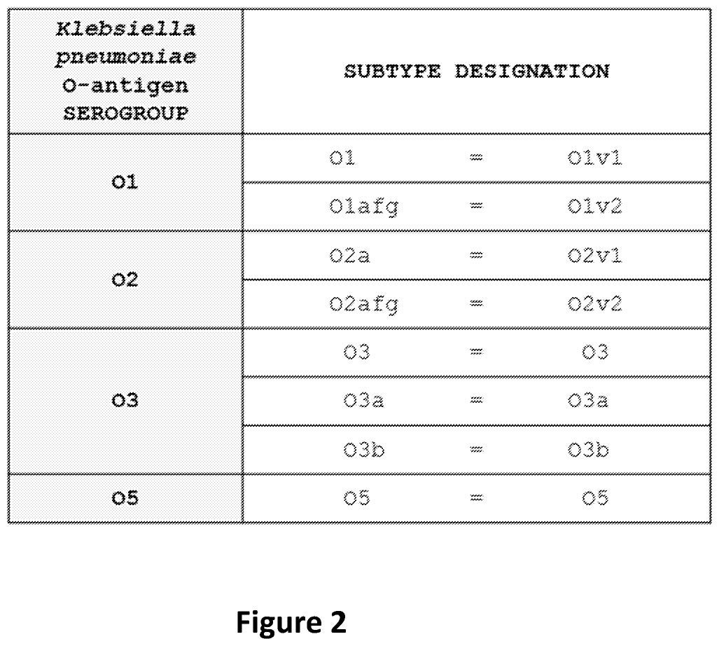

K. pneumoniae O-antigen polysaccharide diversity. The O1 and O2 K. pneumoniae serogroups are characterized as galactans. Galactans are homopolymers of galactose monosaccharides. The O3 and O5 serogroups are characterized as mannans Mannans are homopolymers of mannose monosaccharides. Both galactans and mannans are assembled on top of the reducing end sugar (N-acetylglucosamine (GlcNAc)) (Clarke, B. R. et al., 1995). Previous work has shown that the O1 and O2 serogroups contain a shared backbone structural motif termed the O2a antigen, which is also referred to as D-galactan I (Clarke, B. R. et al., 2018) or the O2v1 antigen according to the Kaptive program (Wick, R. R., Heinz, E., Holt, K. E. & Wyres, K. L., 2018). Six proteins encoded by the genes located in the rfb cluster (wzm-wbbO) (Clarke, B. R. et al., 1995; Clarke, B. R. & Whitfield, C., 1992; Guan, S., Clarke, A. J. & Whitfield, C., 2001) are necessary and sufficient for the assembly of the O2a (O2v1, D-galactan I) backbone in E. coli (Clarke, B. R. et al., 2018). The O2a antigen can be directly modified to form structurally and antigenically unique variants termed the O2afg antigen, also referred to as D-Galactan III (Clarke, B. R. et al., 2018; Szijarto, V. et al., 2016) or the O2v2 antigen according to the Kaptive program (Wick, R. R., Heinz, E., Holt, K. E. & Wyres, K. L., 2018) or the O2aeh antigen (Clarke, B. R. et al., 2018), which to date is not yet named in the Kaptive program. The O2a antigen can be further modified into the O1 antigen, also known as D-galactan II or the O1v1 antigen according to the Kaptive program. A single gene, wbbY, when combined with the rfb gene cluster is necessary and sufficient for the production of the O1 serotype (Hsieh, P. F. et al., 2014; Kelly, S. D. et al., 2019). To generate the O1afg serotype, also known as the O1v2 serotype according to Kaptive, the presence of both the wbbY and gmlABC genes are required in addition to rfb gene cluster (Stojkovic, K. et al., 2017). The mannans (O3 and O5 groups) on the other hand differ in the number of mannose residues per repeat unit, the linkages between mannose residues, as well as a terminal cap on the non-reducing end of the polysaccharide (Greenfield, L. K. et al., 2012; Vinogradov, E. et al., 2002). Six of the eight genes required to assemble the O3 and O5 groups share high homology to each other; however, differences in the coding sequence of the mannosyltransferase (WbdA) result in mannans with different number of mannose residues in the repeat unit and different linkages resulting in distinct serotypes (Guachalla, L. M. et al., 2017). Further, the O5 O-antigen is capped with a methyl group (Vinogradov, E. et al., 2002); whereas, the O3 (O3, O3a and O3b) O-antigens are capped with methylphosphate (Kubler-Kielb, J., Whitfield, C., Katzenellenbogen, E. & Vinogradov, E., 2012). FIG. 1 shows some of the carbohydrate O-repeating structures of the O-antigen polysaccharides of K. pneumoniae. FIG. 2 shows O-antigen serogroup and subtype nomenclatures previously published by multiple groups (Clarke, B. R. et al., 2018; Wick, R. R., Heinz, E., Holt, K. E. & Wyres, K. L., 2018; Szijarto, V. et al., 2016; Stojkovic, K. et al., 2017; Guachalla, L. M. et al., 2017).

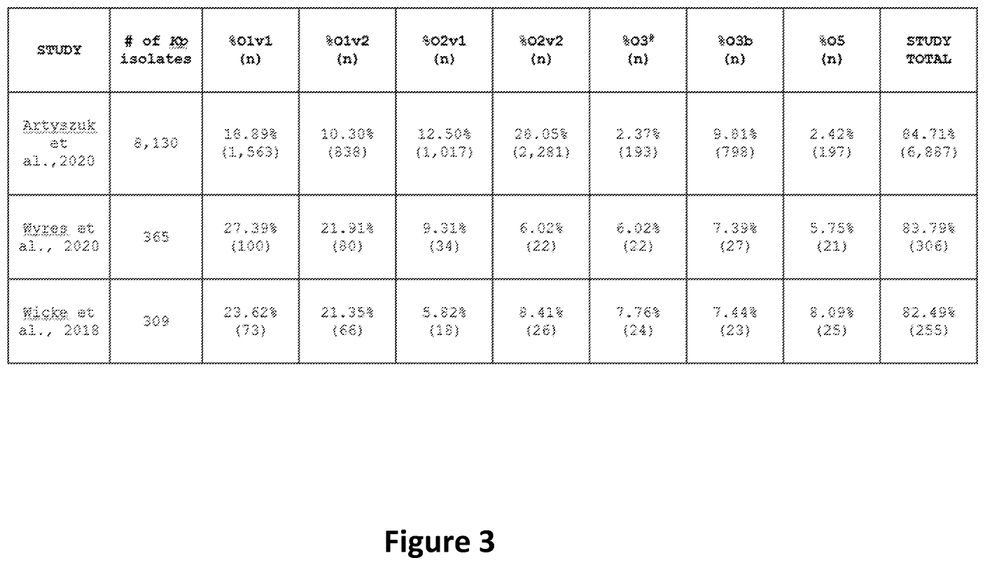

Recently identified subtypes are prevalent among K. pneumoniae isolates as determined by the Kaptive program. The Kaptive program is used to bioinformatically assign a putative O-antigen or capsular polysaccharide serotype using only the DNA sequence of a particular Klebsiella pneumoniae isolate (Wick, R. R., Heinz, E., Holt, K. E. & Wyres, K. L., 2018). A few studies have used Kaptive to assign the O-antigen polysaccharide types to large sets of K. pneumoniae genome sequences. The K. pneumoniae isolates come from diverse collections across multiple clinical sites and geographic regions. FIG. 3 shows the results from three studies (Wick, R. R., Heinz, E., Holt, K. E. & Wyres, K. L., 2018; Artyszuk, D. et al., 2020; Wyres, K. L. et al., 2020). in particular, which demonstrate that seven O-antigen serotypes (O1v1, O1v2, O2v1, O2v2, O3, O3b and O5) account for >80% of all K. pneumoniae isolates when typed by the Kaptive program. Collectively, this indicates that a vaccine targeting these seven O-types of K. pneumoniae could prevent the majority of K. pneumoniae infections.

Bioconjugate. Provided herein is a bioconjugate comprising a K. pneumoniae O-antigen covalently linked to a fusion protein. In certain embodiments, the fusion protein comprises a ComP protein or a glycosylation tag fragment thereof as described in detail elsewhere herein. In certain embodiments, the O-antigen has not been derivatized by: i) being subject to oxidation/reduction procedures; ii) activated with 1-Cyano-4-Dimethylaminopyridine Tetrafluoroborate (CDAP); iii) the addition of primary amines; and/or iv) the addition of diamine spacer molecules. In certain embodiments, the O-antigen has not been derivatized. For the purposes of this disclosure, “has not been derivatized” or “underivatized” does not include the covalent attachment of the O-antigen to the fusion protein. Thus, in certain embodiments, the O-antigen is a native O-antigen. In certain embodiments, the bioconjugate is immunogenic. In certain embodiments, the K. pneumoniae O-antigen is selected from the group consisting of O1, O2, O3, O4, O5, O7, O8 and O12; in certain embodiments, the K. pneumoniae O-antigen is selected from the group consisting of O1v1, O1v2, O2v1, O2v2, O3, O3a, and O3b; and/or in certain embodiments, the K. pneumoniae O-antigen is selected from the group consisting of O1afg, O2afg, O2aeh, and O2ac (see, e.g., FIG. 2 for K. pneumoniae O-antigen nomenclature).

In certain embodiments, the fusion protein of the bioconjugate comprises a ComP protein or a glycosylation tag fragment thereof attached to a heterologous carrier protein. In certain embodiments, the ComP protein or a glycosylation tag fragment thereof is attached to the heterologous carrier protein via an amino acid linker. The ComP protein or a glycosylation tag fragment can be attached either C-terminal or N-terminal to the heterologous carrier protein. In certain embodiments, the ComP protein or a glycosylation tag fragment thereof is located in the fusion protein C-terminal to the heterologous carrier protein. In certain embodiments, the ComP protein or a glycosylation tag fragment thereof is located in the fusion protein N-terminal to the heterologous carrier protein. Further, in certain embodiments, the fusion protein comprises a signal peptide.

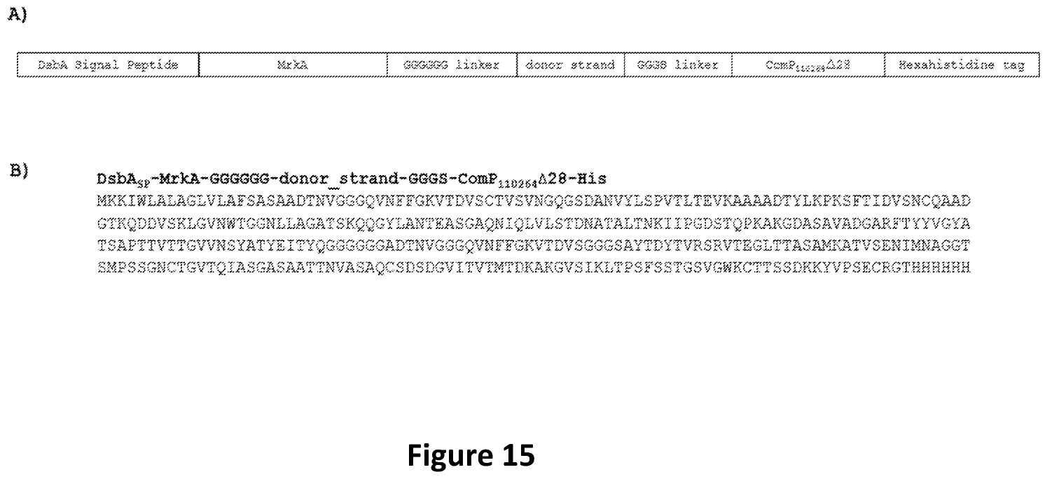

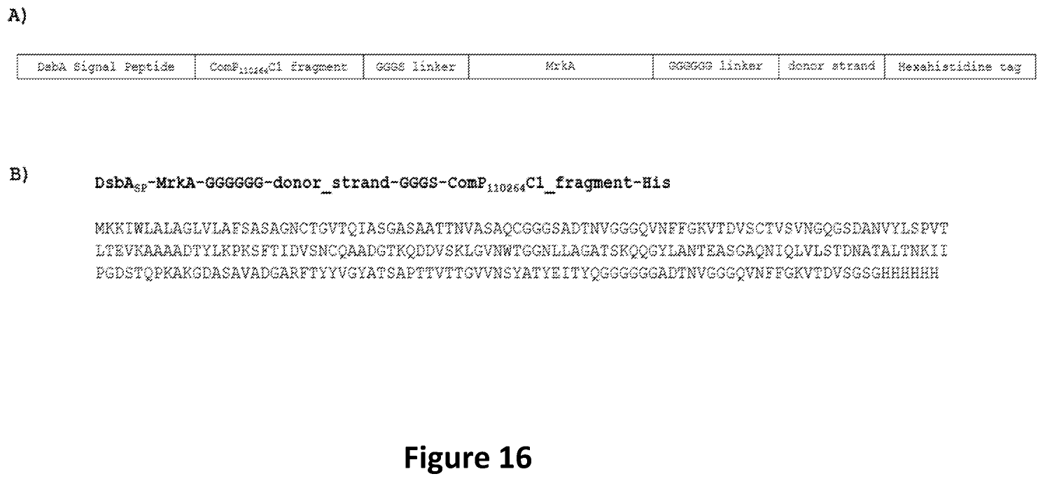

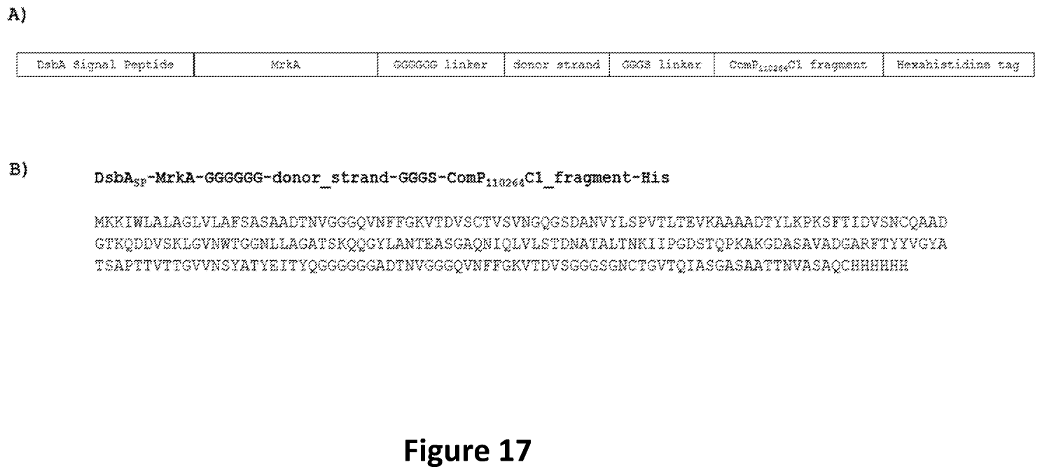

In certain embodiments, the fusion protein of the bioconjugate comprises a Pseudomonas aeruginosa exotoxin A (EPA) carrier protein, a CRM₁₉₇ carrier protein, a tetanus toxin C fragment carrier protein, or a K. pneumoniae MrkA carrier protein. For example, a MrkA carrier protein comprising a modified MrkA variant that is self-complemented by translationally fusing a hexaglycine linker and a duplicated MrkA N-terminal donor strand to the C-terminus of the MrkA protein. In certain embodiments, the MrkA carrier protein comprises a native MrkA signal peptide or comprises a DsbA protein signal peptide in place of the MrkA native signal peptide. In certain embodiments, the MrkA carrier protein comprises a glycine-glycine-glycine-serine linker linking it to ComP protein or a glycosylation tag fragment thereof (see, e.g., FIG. 15, FIG. 16, and FIG. 17).

In certain embodiments, the glycosylation tag fragment of ComP comprises or consists of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2]; SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO: 58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7]; SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11]. In certain embodiments, the ComP glycosylation tag does not comprise a methionine residue in a position corresponding to the conserved methionine residue at position 104 of SEQ ID NO: [2] (ComP110264: ENV58402.1). In certain embodiments, the amino acid sequence of the ComP glycosylation tag does not extend in the C-terminus direction beyond the amino acid residue corresponding to position 103 of SEQ ID NO: [2](ComP110264: ENV58402.1).

In certain embodiments, the fusion protein of the bioconjugate comprises SEQ ID NO: 122 (DsbASP-ComP₁₁₀₂₆₄C1_fragment-GGGS-MrkA-GGGGGG-donor_strand) or SEQ ID NO: 124 (DsbASP-MrkA-GGGGGG-donor_strand-GGGS-ComP₁₁₀₂₆₄C1_fragment). One of ordinary skill in the art would recognize that the addition of a His tag can aid in the purification process. Thus, in certain embodiments, the fusion protein of the bioconjugate comprises SEQ ID NO: 121 (DsbASP-ComP₁₁₀₂₆₄C1_fragment-GGGS-MrkA-GGGGGG-donor_strand-His) or SEQ ID NO: 123 (D sbASP-MrkA-GGGGGG-donor_strand-GGGS -ComP₁₁₀₂₆₄C1_fragment-His).

In certain embodiments, the bioconjugate of this disclosure is a conjugate vaccine. That is, this disclosure provides for a bioconjugate for use as a conjugate vaccine.

Methods of producing a bioconjugate can be found elsewhere herein. In certain embodiments, the bioconjugate is produced in vivo such as in a bacterial cell, for example, the bioconjugate is produced in E. coli.

Conjugate vaccine composition. Provided for herein is a conjugate vaccine composition comprising at least one bioconjugate of this disclosure as described in detail elsewhere herein. In certain embodiments, the conjugate vaccine composition is a multivalent vaccine. In certain embodiments, a multivalent vaccine comprises at least two, three, four, five, six, seven, eight, nine, or ten of the bioconjugates, each comprising a different K. pneumoniae O-antigen. In certain embodiments, the conjugate vaccine composition is a multivalent vaccine comprising, consisting, or consisting essentially of seven of the bioconjugates, each comprising a different K. pneumoniae O-antigen. For example, in certain embodiments, the conjugate vaccine composition comprises: (i) a bioconjugate comprising an O1v1 antigen; (ii) a bioconjugate comprising an O1v2 antigen; (iii) a bioconjugate comprising an O2v1 antigen; (iv) a bioconjugate comprising an O2v2 antigen; (v) a bioconjugate comprising an O3 antigen; (vi) a bioconjugate comprising an O3b antigen; and (vii) a bioconjugate comprising an O5 antigen.

In certain embodiments, the conjugate vaccine composition further comprises an adjuvant.

Fusion protein. Provided for herein is a fusion protein comprising ComP or a glycosylation tag fragment thereof and a heterologous carrier protein. In certain embodiments, the ComP protein or a glycosylation tag fragment thereof is attached to the carrier protein via an amino acid linker. The ComP protein or a glycosylation tag fragment can be attached either C-terminal or N-terminal to the carrier protein. In certain embodiments, the ComP protein or a glycosylation tag fragment thereof is located in the fusion protein C-terminal to the carrier protein. In certain embodiments, the ComP protein or a glycosylation tag fragment thereof is located in the fusion protein N-terminal to the carrier protein. Further, in certain embodiments, the fusion protein comprises a signal peptide.

In certain embodiments, the heterologous carrier protein of the fusion protein is a Pseudomonas aeruginosa exotoxin A (EPA) carrier protein, a CRM₁₉₇ carrier protein, a tetanus toxin C fragment carrier protein, or a K. pneumoniae MrkA carrier protein. For example, a MrkA carrier protein comprising a modified MrkA variant that is self-complemented by translationally fusing a hexaglycine linker and a duplicated MrkA N-terminal donor strand to the C-terminus of the MrkA protein. In certain embodiments, the MrkA carrier protein comprises a native MrkA signal peptide or comprises a DsbA protein signal peptide in place of the MrkA native signal peptide. In certain embodiments, the MrkA carrier protein comprises a glycine-glycine-glycine-serine linker linking it to ComP protein or a glycosylation tag fragment thereof

In certain embodiments, the fusion protein is covalently linked to a K. pneumoniae O-antigen. In certain embodiments, the O-antigen has not been derivatized by: i) being subject to oxidation/reduction procedures; ii) activated with 1-Cyano-4-Dimethylaminopyridine Tetrafluoroborate (CDAP); iii) the addition of primary amines; and/or iv) the addition of diamine spacer molecules. In certain embodiments, the O-antigen has not been derivatized. For the purposes of this disclosure, “has not been derivatized” or “underivatized” does not include the covalent attachment of the O-antigen to the fusion protein. Thus, in certain embodiments, the O-antigen is a native O-antigen. In certain embodiments, the fusion protein comprising an O-antigen is immunogenic. In certain embodiments, the K. pneumoniae O-antigen is selected from the group consisting of O1, O2, O3, O4, O5, O7, O8 and O12; in certain embodiments, the K. pneumoniae O-antigen is selected from the group consisting of O1v1, O1v2, O2v1, O2v2, O3, O3a, and O3b; and/or in certain embodiments, the K. pneumoniae O-antigen is selected from the group consisting of O1afg, O2afg, O2aeh, and O2ac.

In certain embodiments, the glycosylation tag fragment of ComP comprises or consists of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2]; SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO: 58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7]; SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11]. In certain embodiments, the ComP glycosylation tag does not comprise a methionine residue in a position corresponding to the conserved methionine residue at position 104 of SEQ ID NO: [2] (ComP110264: ENV58402.1). In certain embodiments, the amino acid sequence of the ComP glycosylation tag does not extend in the C-terminus direction beyond the amino acid residue corresponding to position 103 of SEQ ID NO: [2] (ComP110264: ENV58402.1).

In certain embodiments, the fusion protein comprises SEQ ID NO: 122 (DsbASP-ComP₁₁₀₂₆₄C1_fragment-GGGS-MrkA-GGGGGG-donor_strand) or SEQ ID NO: 124 (DsbASP-MrkA-GGGGGG-donor_strand-GGGS-ComP₁₁₀₂₆₄ C1_fragment). One of ordinary skill in the art would recognize that the addition of a His tag can aid in the purification process. Thus, in certain embodiments, the fusion protein of the bioconjugate comprises SEQ ID NO: 121 (DsbASP-ComP₁₁₂₆₄C1_fragment-GGGS-MrkA-GGGGGG-donor_strand-His) or SEQ ID NO: 123 (DsbASP-MrkA-GGGGGG-donor_strand-GGGS -ComP₁₁₀₂₆₄C1_fragment-His).

Method of producing a bioconjugate. Provided for herein is a method, as described in greater detail elsewhere herein, of producing a bioconjugate of this disclosure. In certain embodiments, the method comprises covalently linking a K. pneumoniae O-antigen to a fusion protein with a Pg1S oligosaccharyltransferase (OTase), wherein the fusion protein comprises a ComP protein or a glycosylation tag fragment thereof. In certain embodiments, the ComP protein or glycosylation tag fragment thereof is linked to a heterologous carrier protein.

Also provided for herein is a method of inducing a host immune response against K. pneumoniae comprising administering to a subject in need of the immune response an effective amount of the bioconjugate, fusion protein, and/or conjugate vaccine composition of this disclosure. In certain embodiments, the subject is a mammal. In certain embodiments, the subject is a human. In certain embodiments, the immune response is an antibody response. In certain embodiments, the immune response is selected from the group consisting of an innate response, an adaptive response, a humoral response, an antibody response, cell mediated response, a B cell response, a T cell response, cytokine upregulation or downregulation, immune system cross-talk, and a combination of two or more of said immune responses. In certain embodiments, the immune response is selected from the group consisting of an innate response, a humoral response, an antibody response, a T cell response, and a combination of two or more of said immune responses.

Also provided for herein is a method of preventing or treating a K. pneumoniae infection in a subject comprising administering to a subject in need thereof an effective amount of the bioconjugate, fusion protein, and/or conjugate vaccine composition of this disclosure. In certain embodiments, the subject is a mammal In certain embodiments, the subject is a human.

Also provided for herein is the use of the bioconjugate, fusion protein and/or the conjugate vaccine composition of this disclosure to induce a host immune response against K. pneumoniae, prevent a K. pneumoniae infection, and/or treat a K. pneumoniae infection.

Also provided for herein is a method of producing a conjugate vaccine against K. pneumoniae infection comprising: (a) isolating the bioconjugate and/or fusion protein of this disclosure; and (b) combining the isolated bioconjugate and/or fusion protein with an adjuvant.

Pg1S oligosaccharyltransferase (OTase) and ComP. The oligosaccharyltransferase Pg1S—previously referred to as Pg1L by Schulz et al. (PMID23658772) and Pg1L_{Comp} by Harding et al., 2015 (PMID 26727908)—was only recently characterized as a functional OTase (Schulz, B. L. et al. PLoS One 8, e62768 (2013)). Subsequent mass spectrometry studies on total glycopeptides demonstrated that Pg1S does not act as a general Pg1L-like OTase, glycosylating multiple periplasmic and outer membrane proteins (Harding, C. M. et al. Mol Microbiol 96, 1023-1041 (2015)). In fact, the genome of A. baylyi ADP1 encodes for two OTase, a Pg1L-like ortholog (UniProtKB/Swiss-Prot: Q6FFS6.1), which acts as the general OTase and Pg1S (UniProtKB/Swiss-Prot: Q6F7F9.1), which glycosylates a single protein, ComP (Harding, C. M. et al. Mol Microbiol 96, 1023-1041 (2015)).

ComP is orthologous to type IV pilin proteins, like PiIA from Pseudomonas aeruginosa and PilE from Neisseria meningiditis, both of which are glycosylated by the OTases TfpO (Castric, P. Microbiology 141 (Pt 5), 1247-1254 (1995)) and Pg1L (Power, P. M. et al. Mol Microbiol 49, 833-847 (2003)), respectively. Although TfpO and Pg1L also glycosylate their cognate pilins at serine residues, the sites of glycosylation differ between each system. TfpO glycosylates its cognate pilin at a C-terminal serine residue (Comer, J. E., Marshall, M. A., Blanch, V. J., Deal, C. D. & Castric, P. Infect Immun 70, 2837-2845 (2002)), which is not present in ComP. Pg1L glycosylates PilE at an internal serine located at position 63 (Stimson, E. et al. Mol Microbiol 17, 1201-1214 (1995)). ComP also contains serine residues near position 63 and the surrounding residues show moderate conservation to PilE from N. meningiditis. Comprehensive glycopeptide analysis, however, revealed this serine and the surrounding residues were not the site of glycosylation in ComP. Pg1S glycosylates ComP at a single serine residue located at position corresponding to the conserved serine at position 84 of ComP_{ADP1}: AAC4588631 (SEQ ID NO: 1) (also corresponding to the conserved serine at position 82 of ComP₁₁₀₂₆₄: ENV58402.1 (SEQ ID NO: 2)), which is a novel glycosylation site not previously found within the type IV pilin superfamily. The ability of Pg1S to transfer polysaccharides containing glucose as the reducing end sugar coupled with the identification of a novel site of glycosylation within the pilin superfamilies demonstrates that Pg1S is a functionally distinct OTase from PglL and TfpO.

PgIS, but not PgIB or PgIL, transferred polysaccharides containing glucose at their reducing end to the acceptor protein ComP. Two classes of OTases, PglB and PglL, have previously been employed for in vivo conjugation (Feldman, M.F. et al. Proc Natl Acad Sci USA 102, 3016-3021 (2005); Faridmoayer, A., Fentabil, M. A., Mills, D. C., Klassen, J. S. & Feldman, M. F. J Bacteriol 189, 8088-8098 (2007)). PglB, the first OTase described, preferentially transfers glycans containing an acetamido-group at the C-2 position of the reducing end (i.e. N-acetylglucosamine), as it is believed to play a role in substrate recognition (Wacker, M. et al. Proc Natl Acad Sci USA 103, 7088-7093 (2006)). However, polysaccharides with galactose (Gal) at the reducing end, such as the S. enterica Typhimurium O antigen, can be transferred by an engineered Pg1B variant (Ihssen, J. et al. Open Biol 5, 140227 (2015)). The second described OTase, Pg1L from N. meningiditis, has more relaxed substrate specificity than Pg1B, naturally transferring polysaccharides with an acetamido-group at the C-2 position as well as polysaccharides containing galactose (Gal) at the reducing end (Faridmoayer, A., Fentabil, M. A., Mills, D. C., Klassen, J. S. & Feldman, M. F. J Bacteriol 189, 8088-8098 (2007); Pan, C. et al. MBio 7 (2016)). However, there is no evidence available for PglB or PglL mediated transfer of polysaccharides containing glucose (Glc) at the reducing end, which is of particular interest given that the majority of pneumococcal CPSs contain glucose at the reducing end (Geno, K. A. et al. Clin Microbiol Rev 28, 871-899 (2015)). The ability of PglB and PglL to transfer the pneumococcal serotype 14 capsular polysaccharide (CPS14) to their cognate glycosylation targets, AcrA (Wacker, M. et al. Science 298, 1790-1793 (2002)) and DsbA (Vik, A. et al. Proc Natl Acad Sci USA 106, 4447-4452 (2009)), respectively, was tested. Both acceptor proteins were expressed; however, no evidence for CPS14 glycosylation to either acceptor protein was observed (WO/2020/131236).

Acinetobacter species have been described as containing three O-linked OTases; a general Pg1L OTase responsible for glycosylating multiple proteins, and two pilin-specific OTases (Harding, C. M. Mol Microbiol 96, 1023-1041 (2015)). The first pilin-specific OTase is an ortholog of TfpO (also known as PilO) and is not employed for in vivo conjugation systems due to its inability to transfer polysaccharides with more than one repeating unit (Faridmoayer, A., Fentabil, M. A., Mills, D. C., Klassen J. S. & Feldman, M. F. J Bacteriol 189, 8088-8098 (2007)). The second pilin specific OTase, PglS glycosylates a single protein, the type IV pilin ComP 28 . A bioinformatic analysis indicated that PglS is the archetype of a distinct family of OTases. Given that PglS represents a new class of O-OTase, its ability to transfer pneumococcal CPS14 to its cognate acceptor protein, ComP (Harding, C. M. et al. Mol Microbiol 96, 1023-1041 (2015)) was tested. Co-expression of the CPS14 biosynthetic locus in conjunction with PglS and a hexa-his (SEQ ID NO: 114) tagged variant of ComP resulted in a typical ladder-like pattern of bands compatible with protein glycosylation when analyzed via western blotting (WO/2020/131236). The higher molecular weight, modal distribution of signals is indicative of protein glycosylation with repeating glycan subunits of increasing molecular weight. Together, these results indicate that, unlike the previously characterized OTases, PglS is able to transfer polysaccharides with glucose at the reducing end.

There are more than 90 serotypes of S. pneumoniae (Geno, K. A. et al. Clin Microbiol Rev 28, 871-899 (2015)). Many increasingly prevalent serotypes, like serotypes 8, 22F, and 33F are not included in currently licensed vaccines. Therefore, the versatility was tested of PglS to generate a multivalent pneumococcal bioconjugate vaccine composition against two serotypes included in Prevnar 13 (serotype 9V and 14) and one serotype not included (serotype 8) (Package Insert-Prevnar 13 FDA, on the world wide web at fda.gov/downloads/BiologicsBloodVaccines/Vaccines/ApprovedProducts/UCM201669.pdf)). Importantly, all of three of these capsular polysaccharides contain glucose as the reducing end sugar (Geno, K. A. et al. Clin Microbiol Rev 28, 871-899 (2015)). Western blot analysis of affinity purified proteins from whole cells co-expressing PglS, a hexa-his (SEQ ID NO: 114) tagger ComP variant, and either CPS8, CPS9V, or CPS14 resulted in the generation CPS-specific bioconjugates (WO/2020/131236). Moreover, antisera specific to either the CPS8, CPS9V, or CPS14 antigens also reacted to the anti-His reactive bands, indicating that ComP-His was glycosylated with the correct polysaccharides. To confirm that the material purified was not contaminated with lipid-linked polysaccharides, the samples were treated with proteinase K and observed a loss of signal when analyzed via western blotting, confirming that the bioconjugates were proteinaceous.

Therefore, it was demonstrated that PglS can transfer S. pneumoniae polysaccharides to ComP, wherein PglB and PglL could not. Specifically, PglS is the only OTase in the known universe capable of transferring polysaccharides with glucose at the reducing end. In certain embodiments, Pg1S can be used to transfer any lipid-linked oligosaccharide or polysaccharide (collectively referred to herein as “oligo- or polysaccharide”) containing glucose at the reducing end to ComP or a fusion protein containing a fragment of ComP.

PglS can transfer capsular polysaccharides of Klebsiella to ComP. Klebsiella pneumonia (K. pneumoniae), a Gram negative opportunistic human pathogen, produces a capsular polysaccharide known to be important for virulence. To date at least 79 antigenically distinct capsular polysaccharides have been described for Klebsiella species (Pan, Y. J. et al. Sci Rep 5, 15573 (2015)). Furthermore, K. pneumoniae is known to produce at least 59 of the 77 capsular polysaccharides, more than half of which contain glucose as the reducing end sugar (Pan, Y. J. et al. Sci Rep 5, 15573 (2015)). To determine if PglS could transfer K. pneumoniae capsular polysaccharides to ComP, the genes encoding for the proteins required for the synthesis of either the K1 or the K2 capsular polysaccharides were cloned into the IPTG inducible pBBR1MCS-2 vector (Kovach, M. E. et al. Gene 166, 175-176 (1995)). The K1 capsule gene locus was cloned from K. pneumoniae NTUH K-2044, a previously characterized K1 capsule producing strain (Wu, K. M. et al. J Bacteriol 191, 4492-4501 (2009)). The K2 capsule gene locus was cloned from K. pneumoniae 52.145, a previously characterized K2 capsule producing strain (Lery, L. M. et al. BMC Biol 12, 41 (2014)). The K1 or the K2 capsular polysaccharide expressing plasmids were then individually introduced into E. coli co-expressing PglS OTase and the acceptor protein ComP from a separate plasmid vector. To enhance expression of K1 and K2 specific polysaccharides, the K. pneumoniae transcriptional activator rmpA from K. pneumoniae NTUH K-2044 was subsequently cloned into pACT3 (Dykxhoorn, D. M., St Pierre, R. & Linn, T. Gene 177, 133-136 (1996)), a low copy, IPTG inducible vector as it has previously been characterized as a regulator of capsule in K. pneumoniae (Arakawa, Y. et al. Infect Immun 59, 2043-2050 (1991)); Yeh, K. M. et al. J Clin Microbiol 45, 466-471 (2007)). Introduction of the rmpA gene into E. coli strains co-expressing Pg1S and hexa-his (SEQ ID NO: 114) tagged ComP variant and either the K1 or K2 capsular polysaccharides from K. pneumoniae, resulted robust expression and detection of higher molecular ComP bioconjugates as indicated by the typical ladder-like pattern of bands compatible with protein glycosylation when analyzed via western blotting (WO/2020/131236). The modal distribution of signals is indicative of protein glycosylation with repeating glycan subunits of increasing molecular weight. Thus collectively, Pg1S was able to glycosylate ComP with the K1 and K2 capsular polysaccharides from K. pneumonia. Increased efficiency of conjugation was observed with co-expression of the transcriptional activator rmpA from K. pneumoniae.

PglS can transfer K. pneumoniae polysaccharides to ComP. Given that most K. pneumoniae capsular polysaccharides contain glucose as the reducing end sugar, the only other commercially licensed OTases (PglB and PglL) should be unable to generate conjugate vaccines using these polysaccharides. Moreover, co-expression of the transcriptional activator, RmpA, with the capsule gene cluster enhanced capsule expression to detectably levels. In certain embodiments, the method for producing Klebsiella conjugates can be used to generate a pan Klebsiella conjugate vaccine composition encompassing all serotypes—including other species such as K. varricola, K. michiganensis, and K. oxytoca.

Mass spectrometry and site directed mutagenesis confirm PglS is an O-linked OTase and reveal that ComP is glycosylated at a serine residue corresponding to position 84 of ComP_{ADP1}. N-glycosylation in bacteria generally occurs within the sequon D-X-N-S-T (SEQ ID NO: 21), where X is any amino acid but proline (Kowarik, M. et al. EMBO J 25, 1957-1966 (2006)). On the contrary, O-glycosylation does not seem to follow a defined sequon. Most O-glycosylation events in bacterial proteins occur in regions of low complexity (LCR), rich in serine, alanine, and proline (Vik, A. et al. Proc Natl Acad Sci USA 106, 4447-4452 (2009)). Alternatively, some pilins are O-glycosylated at a C-terminal serine residue (Comer, J. E., Marshall, M. A., Blanch, V. J., Deal, C. D. & Castric, P. Infect Immun 70, 2837-2845 (2002)). ComP does not appear to have an obvious LCR or a C-terminal serine residue homologous to those found in other pilin like proteins and therefore mass spectrometry was employed to determine the site(s) of glycosylation. Purified CPS14-ComP bioconjugates were subjected to proteolytic digestion, ZIC-HILIC glycopeptide enrichment, and multiple MS analyses. A single glycopeptide consisting of the peptide ISASNATTNVATAT (SEQ ID NO: 22) was identified attached to a glycan that matched the published CPS14 composition (WO/2020/131236; Geno, K. A. et al. Clin Microbiol Rev 28, 871-899 (2015)). To enable confirmation of both the peptide and attached glycan sequences, multiple collision energies regimes were performed to confirm the glycosylation of the semi-GluC derived peptide ISASNATTNVATAT (SEQ ID NO: 22) with a 1378.47 Da glycan corresponding to HexNAc2Hexose6 (WO/2020/131236). Additional glycopeptides were also observed decorated with extended glycans corresponding to up to four tetrasaccharide repeat units (WO/2020/131236).

It was previously shown that Acinetobacter species predominantly glycosylate proteins at serine residues and thus it was hypothesized that either serine (S) 82 or 84—as numbered in SEQ ID NO: 1—was the site of glycosylation (Scott, N. E. et al. Mol Cell Proteomics 13, 2354-2370 (2014)). To determine which serine residue was the site of glycosylation, these serine residues were individually mutated to alanine (A) and the glycosylation status of both mutant proteins was analyzed. For this experiment, the biosynthetic locus for the C. jejuni heptasaccharide was employed as the donor glycan, as glycosylation is readily detectable with the hR6 anti-glycan antisera as well as by an increase in electrophoretic mobility (Schwarz, F. et al. Nat Chem Biol 6, 264-266 (2010)). Wild type hexa-his (SEQ ID NO: 114) tagged ComP was glycosylated with the C. jejuni heptasaccharide as indicated by its increased electrophoretic mobility and co-localization with hR6 antisera signal when co-expressed with Pg1S (WO/2020/131236). MS analysis also confirmed the presence of the C. jejuni heptasaccharide on the identical semi-GluC derived peptide ISASNATTNVATAT (SEQ ID NO: 22) modified by CPS14 (WO/2020/131236). As a negative control, a catalytically inactive Pg1S mutant (H324A) was generated, that when co-expressed with the C. jejuni heptasacchride glycan was unable to glycosylate wild type ComP. Site directed mutagenesis was performed and it was observed that glycosylation of ComP with the C. jejuni heptasaccharide was abolished in the ComP[S84A] mutant, whereas ComP[S82A] was glycosylated at wild-type levels. Together, these results indicate that ComP is singly glycosylated at serine 84 (as numbered in SEQ ID NO: 1) by PglS, which is a unique site that is different than other previously characterized pilin like proteins. This corresponds to serine 82 as numbered in SEQ ID NO: 2.

Bioinformatic features of ComP pilin orthologs. ComP was first described as a factor required for natural transformation in Acinetobacter baylyi ADP1 (Porstendorfer, D., Drotschmann, U. & Averhoff, B. Appl Environ Microbiol 63, 4150-4157 (1997)). In a subsequent study, it was demonstrated that ComP from A. baylyi ADP1 (herein referred to as ComP_{ADp1}) was glycosylated by a novel OTase, Pg1S, located immediately downstream of ComP, and not the general OTase PglL located elsewhere on the chromosome (Harding, C. M. et al. Mol Microbiol 96, 1023-1041 (2015)). The ComP_{ADP1} protein (NCBI identifier AAC45886.1) belongs to a family of proteins called type IV pilins. Specifically, ComP shares homology to type IVa major pilins (Giltner, C. L., Nguyen, Y. & Burrows, L. L. Microbiol Mol Biol Rev 76, 740-772 (2012)). Type IVa pilins share high sequence homology at their N-terminus, which encode for the highly conserved leader sequence and N-terminal alpha helix; however, the C-terminus display remarkable divergences across genera and even within species (Giltner, C. L., Nguyen, Y. & Burrows, L. L. Microbiol Mol Biol Rev 76, 740-772 (2012)). To help differentiate ComP orthologs from other type IVa pilin proteins, such as, PilA from A. baumannii, P. aeruginosa, and Haemophilus influenzae as well as PilE from Neisseria species (Pelicic, V. Mol Microbiol 68, 827-837 (2008)), a BLASTp analysis was performed comparing the primary amino acid sequence of ComP_{ADP1} against all proteins from bacteria in the Acinetobacter genus. Expectedly, many Acinetobacter type IVa pilin orthologs, including ComP A ppi, share high homology at their N-termini; however, very few proteins display high sequence conservation across the entire amino acid sequence of ComP. At least six ComP orthologs were identified based on the presence of the conserved serine at position 84 relative to ComP_{ADP1} as well as a conserved disulfide bond flanking the site of predicted glycosylation connecting the predicted alpha beta loop to the beta strand region (WO/2020/131236; Giltner, C. L., Nguyen, Y. & Burrows, L. L. Microbiol Mol Biol Rev 76, 740-772 (2012)). Furthermore, all six ComP orthologs carry both a pglS homolog immediately downstream of the comP gene as well as a pglL homolog located elsewhere in the chromosome. Together, at least the presence of the conserved serine at position 84, the disulfide loop flanking the site of glycosylation, the presence of a pglS gene immediately downstream of comP, and the presence of a pglL homolog located elsewhere on the chromosome differentiate ComP pilin variants from other type IVa pilin variants.

Therefore, features common to ComP proteins are disclosed herein that identify ComP orthologs in different Acinetobacter species. ComP proteins can be differentiated from other pilins by the presence of the conserved glycosylated serine located at position 84 relative to the ADP1 ComP protein and the presence of a disulfide loop flanking the site of glycosylation. In addition, the presence of a pglS homolog immediately downstream of ComP is an indicator of ComP. Further to be classified as a Pg1S OTase protein rather than a Pg1L OTase protein, the OTase downstream of ComP must display higher sequence conservation with Pg1S (ACIAD3337) when compared to Pg1L (ACIAD0103) in A. baylyi ADP1. It is also evident to one of ordinary skill in the art that in any embodiment disclosed herein, a ComP protein comprises and is capable of being glycosylated on a serine residue corresponding to the conserved serine residue at position 84 of SEQ ID NO: 1 (ComP_{ADP1}: AAC45886.1).

ComP from A. soli CIP 110264 is glycosylated by Pg1S from A. baylyi ADP1. Given the presence of multiple ComP orthologs, whether Pg1S from A. baylyi ADP1 was able to glycosylate a divergent ComP protein was investigated. The ComP protein from A. soli CIP 110264 (ComP₁₁₀₂₆₄) is 71% identical at the amino acid level when compared to ComP_{ADP1}. However, consistent with the features above, ComP₁₁₀₂₆₄ contains the predicted disulfide bridge between the predicted alpha-beta loop and the second beta strand as well as the conserved serine located at position 84 relative to ComP_{ADP1}. Moreover, a Pg1S ortholog can be found immediately downstream of ComP₁₁₀₂₆₄. To determine whether PglS from A. baylyi ADP1 (PglS_{ADP1}) could glycosylate ComP₁₁₀₂₆₄, PglS_{ADP1} was cloned into pACT3 and ComP₁₁₀₂₆₄ into pEXT20 (Dykxhoorn, D. M., St Pierre, R. & Linn, T. Gene 177, 133-136 (1996)) and these plasmids were introduced into E. coli expressing the serotype 8 capsular polysaccharide (CPS8) from S. pneumoniae. Further, the converse experiment was performed by cloning and expressing PglS from A. soli CIP₁₁₀₂₆₄ (PglS₁₁₀₂₆₄) with COMP_{AD1}. PglS₁₁₀₂₆₄ minimally glycosylated its cognate acceptor pilin ComP₁₁₀₂₆₄ as indicated by higher molecular weight ComP pilin variants when compared to whole cell lysates lacking PglS₁₁₀₂₆₄ (WO/2020/131236). Based on western blot analysis, PglS₁₁₀₂₆₄ appeared to not glycosylate ComP_{ADP1}. On the other hand, PglS_{ADP1} efficiently glycosylated both ComP_{ADP1} and ComP₁₁₀₂₆₄ as indicated by the robust increase of His-reactive signals of increasing electrophoretic mobility. Collectively, PglS_{ADP1} appears to be an optimal OTase from heterologous glycosylation in E. coli with a unique ability to cross glycosylate multiple ComP substrates. Thus it was demonstrated that PglS proteins from different Acinetobacter species can glycosylate divergent, non-native ComP sequences.

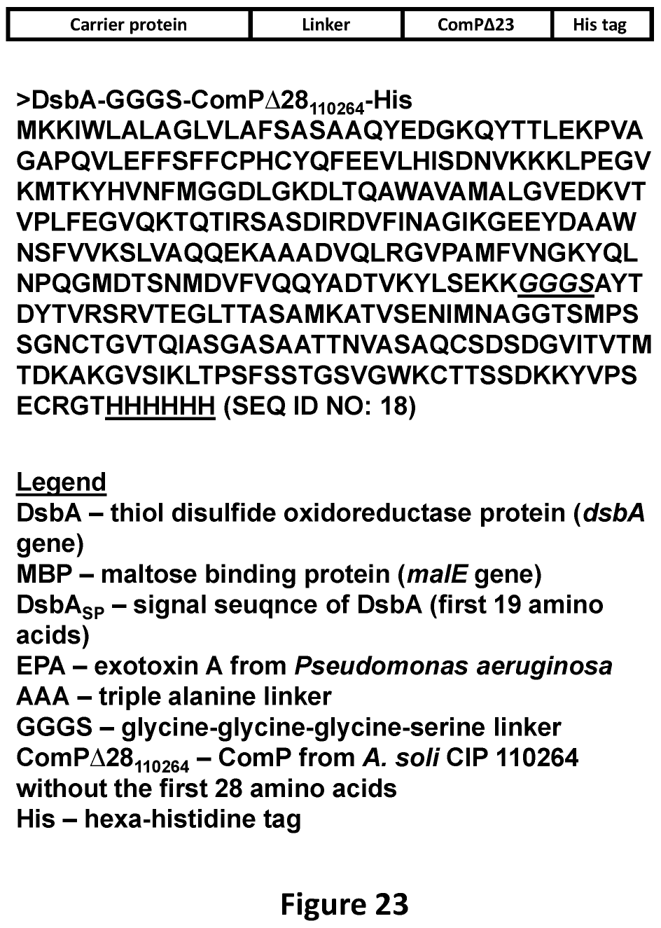

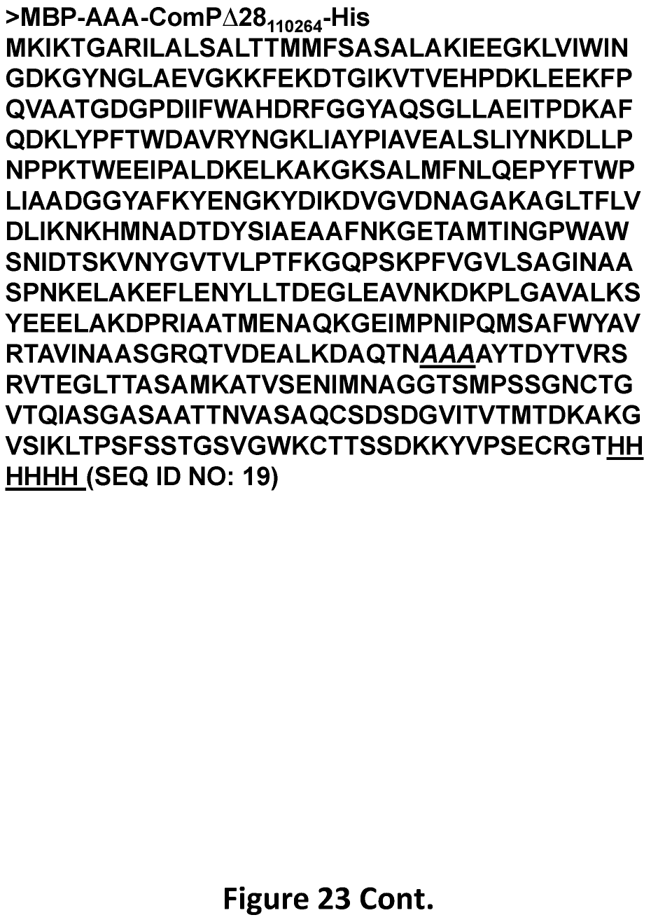

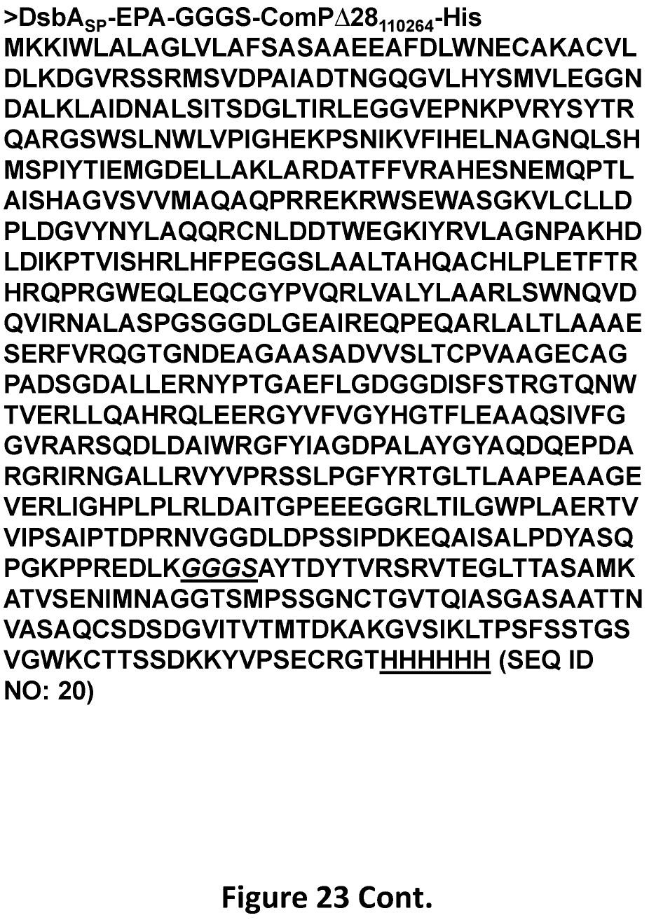

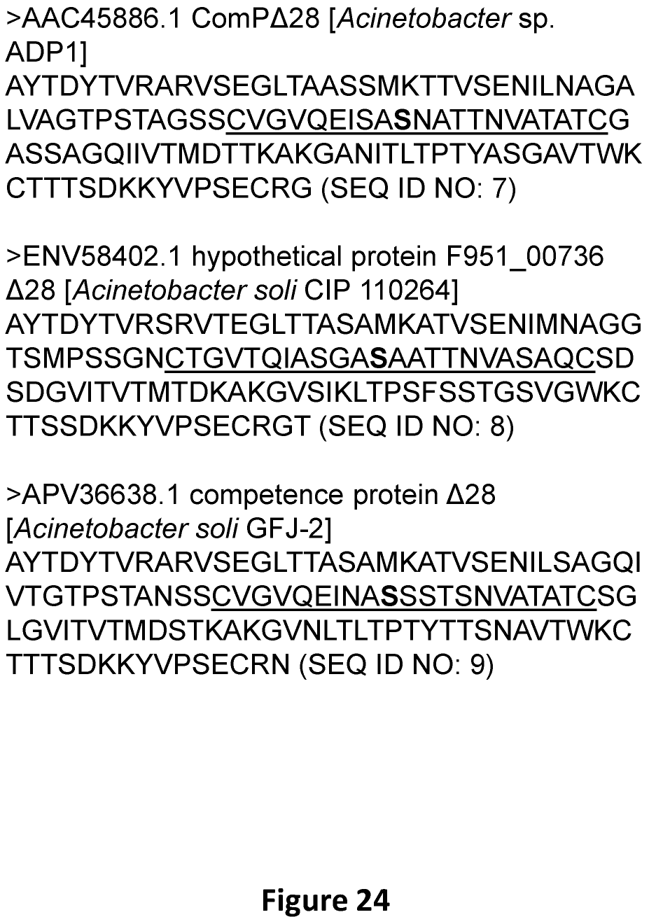

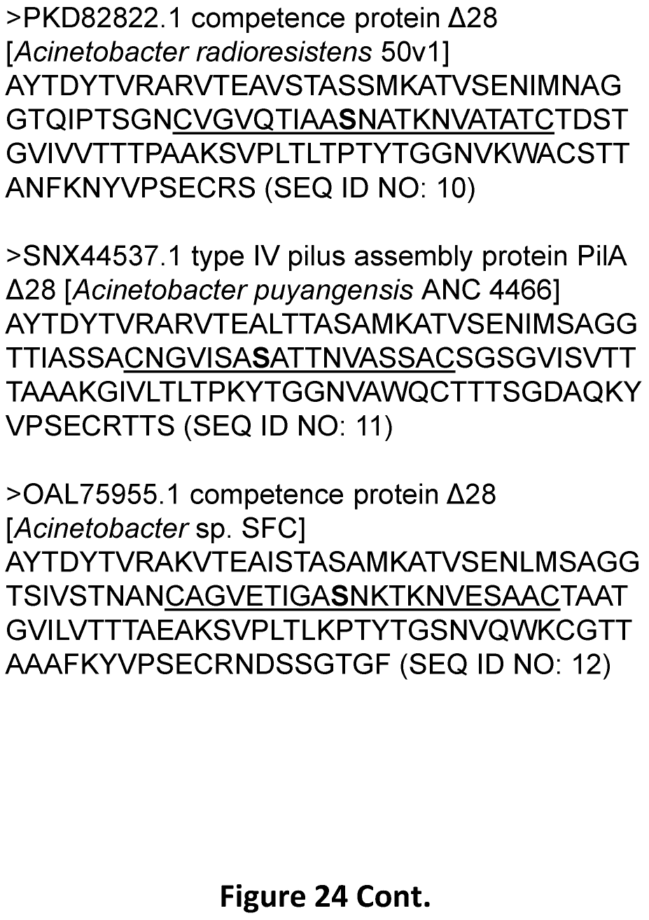

Generation of a soluble, periplasmic fusion protein capable of being glycosylated by PglS. All members of type IVa pilin family are considered membrane proteins as part of their N-terminal alpha helix is embedded within the inner membrane (Giltner, C. L., Nguyen, Y. & Burrows, L. L. Microbiol Mol Biol Rev 76, 740-772 (2012)). Therefore, in order to generate soluble variants of ComP that are able to be glycosylated by PglS, translational fusions were constructed of truncated ComP fragment proteins onto three different carrier proteins. The carrier proteins, DsbA and MalE (also known as maltose binding protein—MBP) from E. coli, were selected as suitable carriers as both have been previously shown to facilitate periplasmic localization and solubility of acceptor proteins fused at their C-termini (Malik, A. Biotech 6, 44 (2016)). Exotoxin A from Pseudomonas aeruginosa (EPA) was also selected as it has been previously shown to act as an immunogenic carrier protein in other conjugate vaccine formulations (Ravenscroft, N. et al. Glycobiology 26, 51-62 (2016)). Fusion proteins consisted of a leader sequence, carrier protein, a short linker peptide, a ComP variant without the first 28 amino acids, and a hexa-histidine tag (SEQ ID NO: 114). The first 28 amino acids of ComP_{ADP1} and ComP₁₁₀₂₆₄ were removed as these amino acids contain the leader sequence as well as the hydrophobic region of the N-terminal alpha helix predicted to be embedded into the inner membrane. Fusion constructs were then introduced into E. coli expressing the pneumococcal serotype 8 capsular polysaccharide (CPS8) and either pACT3 alone or pACT3 carrying pglS₁₁₀₂₆₄ or pglS_{ADP1}. E. coli cells expressing either DsbA-AAA-ComPΔ28₁₁₀₂₆₄ (“AAA” disclosed as SEQ ID NO: 24) or DsbA-GGGS-ComPΔ28₁₁₀₂₆₄ (“GGGS” disclosed as SEQ ID NO: 23) in combination with Pg1S_{ADP1} demonstrated detectable levels of glycosylation as indicated by the modal distribution of his reactive signals of increasing electrophoretic mobility (WO/2020/131236). E. coli cells expressing fusions containing ComPΔ28_{ADP1} did not demonstrate any detectable glycosylation. The same glycosylation pattern was observed for E. coli cells expressing maltose binding protein (MBP) fusions. E. coli cells expressing either MBP-AAA-ComPΔ28₁₁₀₂₆₄ (“AAA” disclosed as SEQ ID NO: 24) or MBP-GGGS-ComPΔ28₁₁₀₂₆₄ (“GGGS” disclosed as SEQ ID NO: 23) in combination with PglS_{ADP1} demonstrated detectable levels of glycosylation as indicated by the modal distribution of anti-His reactive signals; whereas, fusions with ComPA28_{ADP1} were only minimally glycosylated (WO/2020/131236). Lastly, to demonstrate that a previously established carrier protein used for conjugate vaccine formulations could be glycosylated by PglS with the pneumococcal CPS8, a fusion protein was engineered containing the DsbA signal peptide sequence fused to EPA. The ComPA28₁₁₀₂₆₄ peptide was then fused with glycine-glycine-glycine-serine (GGGS; SEQ ID NO: 23) linker to the C-terminus of EPA and tested for glycosylation in the presence and absence of PglS_{ADP1} in both whole cell extracts and in periplasmic extracts. EPA-GGGS-ComPA28₁₁₀₂₆₄ constructs (“GGGS” disclosed as SEQ ID NO: 23) were found to be glycosylated in both the whole cell extract and periplasmic extracts of cells co-expressing the CPS8 glycan and PglS_{ADP1} as indicated by the modal distribution of anti-His reactive signals (WO/2020/131236). No detectable glycosylation was observed in samples lacking a PglS ortholog or in the samples expressing PglS₁₁₀₂₆₄. Collectively, PglS_{ADP1} is an optimal OTase for transferring polysaccharides containing glucose at the reducing end to truncated ComP fusion proteins. Specific amino acid sequences for each fusion construct are shown in FIG. 23.

Immunization with a glycosylated ComP bioconjugate elicits an immune response. T-cell dependent immune responses to conjugate vaccines are characterized by the secretion of high affinity IgG1 antibody (Avci, F. Y., Li, X., Tsuji, M. & Kasper, D. L. Nat Med 17, 1602-1609 (2011)). The immunogenicity of a CPS14-ComP bioconjugate in a murine vaccination model was evaluated. Sera collected from mice vaccinated with a CPS14-ComP bioconjugate had a significant increase in CPS14 specific IgG titers but not IgM titers (WO/2020/131236). Further, secondary HRP-tagged anti-IgG subtype antibodies were employed to determine which of the IgG subtypes had elevated titers. IgG1 titers appeared to be higher than the other subtypes (WO/2020/131236).

Next, a second vaccination trial was performed comparing the immunogenicity of a trivalent CPS8-, CPS9V-, and CPS14-ComP bioconjugate to the current standard of care, PREVNAR 13®. Serotypes 9V and 14 are included in PREVNAR 13® and elevated IgG titers could be seen in PREVNAR 13® immunized mice against these two serotypes (WO/2020/131236). The monovalent immunization against serotype 14 also showed significant induction of serotype specific IgG titers, which were similar to the preliminary immunization (WO/2020/131236). Mice receiving the trivalent bioconjugate, all had elevations in serotype specific IgG titers when compared to control as expected, day 49 sera have shown much more elevated IgG tires for serotypes 8 and 14 compared to serotype 9V. Nevertheless, IgG titers against 9V were still significantly higher than the placebo (WO/2020/131236).

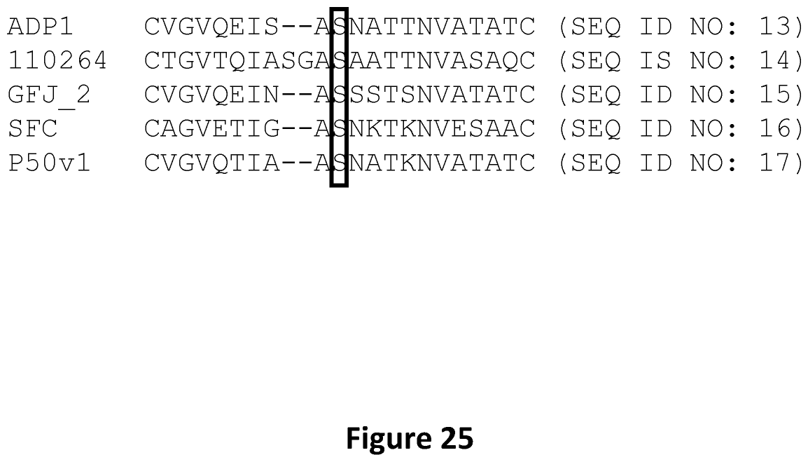

ComP protein is glycosylated on a serine (S) residue. This serine residue is conserved in ComP proteins and corresponds to position 84 of SEQ ID NO: 1 (ComP_{ADP1:} AAC45886.1). This serine residue also corresponds to position 82 of SEQ ID NO: 2 (ComP₁₁₀₂₆₄: ENV58402.1). Thus, in certain aspects, a fusion protein (and thus the bioconjugate) is glycosylated with an oligo- or polysaccharide on a ComP glycosylation tag thereof at a serine residue corresponding to the serine residue at position 84 of SEQ ID NO: 1 (ComP_{ADP1}: AAC45886.1) or corresponding to the serine residue at position 82 of SEQ ID NO: 2. FIG. 25 shows an alignment of a region of ComP sequences including the serine (S) residue (boxed) corresponding to the serine residue at position 84 of SEQ ID NO: 1 (ComP_{ADP1}: AAC45886.1), which is conserved across the ComP sequences. In certain embodiments, in order to be able to be glycosylated, the ComP glycosylation tag comprises both a cysteine residue corresponding to the conserved cysteine residue at position 75 of SEQ ID NO: 1 (ComP_{ADP1}: AAC45886.1) and a cysteine residue corresponding to the conserved cysteine residue at position 95 of SEQ ID NO: 1. Or, similarly described, in certain embodiments, in order to be able to be glycosylated, the ComP glycosylation tag comprises both a cysteine residue corresponding to the conserved cysteine residue at position 71 of SEQ ID NO: 2 (ComP_{ADP1}: AAC45886.1) and a cysteine residue corresponding to the conserved cysteine residue at position 93 of SEQ ID NO: 2.

In certain embodiments of a bioconjugate of this disclosure, the oligo- or polysaccharide comprises a glucose at its reducing end.

One of ordinary skill in the art would recognize that by aligning ComP sequences with SEQ ID NO: 1, (e.g., either full sequences or partial sequences) the conserved serine residue of a non-SEQ ID NO: 1 ComP protein disclosed herein, corresponding to the serine residue at position 84 of SEQ ID NO: 1, can be identified. Further, one of ordinary skill in the art would recognize that by aligning ComP sequences with SEQ ID NO: 1, other residues, regions, and/or features corresponding to residues, regions, and/or features of SEQ ID NO: 1 as referred to herein can be identified in the non-SEQ ID NO: 1 ComP sequence and referenced in relation to SEQ ID NO:1. And, while reference is generally made herein to SEQ ID NO: 1, by analogy, reference can similarly be made to any residue, region, feature and the like of any ComP sequence disclosed herein, for example, in reference to SEQ ID NO: 2.

A ComP protein is a protein that has been identified as ComP protein consistent with the description provided herein. For example, representative examples of ComP proteins include, but are not limited to: AAC45886.1 ComP [Acinetobacter sp. ADP1]; ENV58402.1 hypothetical protein F951_00736 [Acinetobacter soli CIP 110264]; APV36638.1 competence protein [Acinetobacter soli GFJ-2]; PKD82822.1 competence protein [Acinetobacter radioresistens 50v1]; SNX44537.1 type IV pilus assembly protein PilA [Acinetobacter puyangensis ANC 446]; and OAL75955.1 competence protein [Acinetobacter sp. SFC]. In certain embodiments, a ComP protein comprises an amino acid sequence that is at least 50%, 60%, 70%, 75%, 80%, 85%, 90%, 95%, 96%, 97%, 98%, 99%, or 100% identical to SEQ ID NO: 1 (ComP_{ADP1}: AAC45886.1) and contains a serine residue corresponding to the conserved serine residue at position 84 of SEQ ID NO: 1 (ComP_{ADP1}: AAC45886.1). SEQ ID NO: 1 comprises a leader sequence of 28 amino acids. In certain embodiments, a ComP protein comprises an amino acid sequence that is at least 50%, 60%, 70%, 75%, 80%, 85%, 90%, 95%, 97%, 98%, 99%, or 100% identical to SEQ ID NO: 7 (ComPΔ28_{ADP1}), SEQ ID NO: 8 (ComPΔ28₁₁₀₂₆₄), SEQ ID NO: 9 (ComPΔ28_{GFJ-2}), SEQ ID NO: 10 (ComPΔ28_{P50v1}), SEQ ID NO: 11 (ComPΔ28₄₄₆₆), or SEQ ID NO: 12 (ComPΔ28_{SFC}) that do not include the 28 amino acid leader sequence but do contain a serine residue corresponding to the conserved serine residue at position 84 of SEQ ID NO: 1 (ComP_{ADP1}i: AAC45886.1). In certain embodiments, a ComP protein comprises an amino acid sequence that is at least 50%, 60%, 70%, 75%, 80%, 85%, 90%, 95%, 97%, 98%, 99%, or 100% identical to SEQ ID NO: 7 (ComPΔ28_{ADP1}) that does not include the 28 amino acid leader sequence but does contain a serine residue corresponding to the conserved serine residue at position 84 of SEQ ID NO: 1 (ComP_{ADP1}: AAC45886.1). In certain embodiments, the ComP protein comprises SEQ ID NO: 7 (ComPΔ28_{APD1}), SEQ ID NO: 8 (ComPΔ28₁₁₀₂₆₄), SEQ ID NO: 9 (ComPΔ28_{GFJ-2}), SEQ ID NO: 10 (ComPΔ28_{P50v1}), SEQ ID NO: 11 (ComPΔ28₄₄₆₆), or SEQ ID NO: 12 (ComPΔ28_{SFC}). In certain embodiments, the ComP protein is SEQ ID NO: 1 (ComP_{ADP1}: AAC45886.1), SEQ ID NO: 2 (ComP₁₁₀₂₆₄: ENV58402.1), SEQ ID NO: 3 (ComP_{GFJ-2}: APV36638.1), SEQ ID NO: 4 (ComP₅₀ᵥ₁: PKD82822.1), SEQ ID NO: 5 (ComP₄₄₆₆: SNX44537.1), or SEQ ID NO: 6 (ComP_{SFC}: OAL75955.1).

In certain embodiments, the bioconjugate is produced in vivo in a host cell such as by any of the methods of production disclosed herein. In certain embodiments, the bioconjugate is produced in a bacterial cell, a fungal cell, a yeast cell, an avian cell, an algal cell, an insect cell, or a mammalian cell. In certain embodiments, the bioconjugate is produced in a cell free system. Examples of the use of a cell free system utilizing OTases other than PglS can be found in WO2013/067523A1, which in incorporated herein by reference.

It has been discovered that a methionine residue corresponding to the conserved methionine residue at position 104 of SEQ ID NO: 2 (ComP₁₁₀₂₆₄: ENV58402.1) can have an inhibitory effect on glycosylation when present in a ComP glycosylation tag even though the full length ComP protein comprising this methionine residue is glycosylated. Thus, in certain embodiments, the ComP glycosylation tag of this disclosure does not comprise a methionine residue corresponding to the conserved methionine residue at position 104 of SEQ ID NO: 2 (ComP₁₁₀₂₆₄: ENV58402.1). For example, in certain embodiments, such methionine residue in a ComP amino acid sequence is substituted with another amino acid that does not exhibit an inhibitory effect or is deleted from the ComP glycosylation tag amino acid sequence. In certain embodiments, the amino acid sequence of the ComP glycosylation tag does not extend in the C-terminus direction beyond the amino acid residue corresponding to position 103 of SEQ ID NO: 2 (ComP₁₁₀₂₆₄: ENV58402.1). For example, in certain embodiments, the amino acid sequence of the ComP glycosylation tag ends with the residue corresponding to position 93, 94, 95, 96, 97, 98, 99, 100, 101, 102, or 103 of SEQ ID NO: 2 (ComP₁₁₀₂₆₄: ENV58402.1). One of ordinary skill in the art would recognize that a fusion protein comprising a ComP glycosylation tag likewise would not comprise a methionine residue at a position corresponding to or corresponding about to the conserved methionine residue at position 104 of SEQ ID NO: 2 (ComP₁₁₀₂₆₄: ENV58402.1) in relation to the ComP glycosylation tag, even if the methionine residue is attributed to a sequence of the fusion protein not as belonging to the ComP glycosylation tag sequence. For example, in certain embodiments, the fusion protein of the bioconjugate does not comprise, in relationship to the ComP glycosylation tag, a methionine residue at a position that would correspond to or correspond about to the conserved methionine residue at position 104 of SEQ ID NO: 2 (ComP₁₁₀₂₆₄: ENV58402.1). In certain embodiments, the fusion protein of the bioconjugate does not comprise, in relationship to the ComP glycosylation tag, a methionine residue at a position that would correspond to the conserved methionine residue at position 104 of SEQ ID NO: 2 (ComP_{110264:} ENV58402.1).

A ComP glycosylation tag of the current disclosure is generally not a full length ComP protein. In certain embodiments of any ComP glycosylation tag described herein, the ComP glycosylation tag has a length of between 18 and 50 amino acids in length, for example, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 ,49, or 50 amino acids in length. In certain embodiments, the glycosylation tag has length of between 21 and 45 amino acids in length. In certain embodiments, the glycosylation tag has a length of between 23 and 45 amino acids in length.

The ComP glycosylation tag of the current disclosure can be a fragment, a variant, or a variant fragment of a ComP protein as described anywhere herein. In certain embodiments, the ComP protein comprises an amino acid sequence that is at least 50%, 60%, 70%, 75%, 80%, 85%, 90%, 95%, 96%, 97%, 98%, 99%, or 100% identical to SEQ ID NO: 7 (ComPΔ28_{ADP1}), SEQ ID NO: 8 (ComPΔ₂₈₁₁₀₂₆₄), SEQ ID NO: 9 (ComPΔ28_{GFJ-2}), SEQ ID NO: 10 (ComPΔ28_{P50v1}), SEQ ID NO: 11 (ComPΔ₂₈₄₄₆₆), or SEQ ID NO: 12 (ComPΔ28_{SFC}). For example, in certain embodiments, the ComP protein comprises an amino acid sequence that is at least 50%, 60%, 70%, 75%, 80%, 85%, 90%, 95%, 96%, 97%, 98%, 99%, or 100% identical to SEQ ID NO: 7 (ComPΔ28_{ADP1}) or SEQ ID NO: 8 (ComPΔ28₁₁₀₂₆₄). In certain embodiments, the ComP protein comprises SEQ ID NO: 7 (ComPΔ28_{App}1), SEQ ID NO: 8 (ComPΔ28₁₁₀₂₆₄), SEQ ID NO: 9 (ComPΔ28_{GFJ-2}), SEQ ID NO: 10 (ComPΔ28_{P50v1}), SEQ ID NO: 11 (ComPΔ28₄₄₆₆), or SEQ ID NO: 12 (ComPΔ28_{SFC}). Further, in certain embodiments, the ComP protein comprises an amino acid sequence that is at least 50%, 60%, 70%, 75%, 80%, 85%, 90%, 95%, 96%, 97%, 98%, 99%, or 100% identical to SEQ ID NO: 1 (ComP_{ADP1}: AAC45886.1), SEQ ID NO: 2 (ComP₁₁₀₂₆₄: ENV58402.1), SEQ ID NO: 3 (ComP_{GFJ-2}: APV36638.1), SEQ ID NO: 4 (Com_{P501}: PKD82822.1), SEQ ID NO: 5 (ComP₄₄₆₆: SNX44537.1), or SEQ ID NO: 6 (ComP_{SFC}: OAL75955.1). For example, in certain embodiments, the ComP protein comprises an amino acid sequence that is at least 50%, 60%, 70%, 75%, 80%, 85%, 90%, 95%, 96%, 97%, 98%, 99%, or 100% identical to SEQ ID NO: 1 (ComP_{ADP1}: AAC45886.1) or SEQ ID NO: 2 (ComP₁₁₀₂₆₄: ENV58402.1). Further, in certain embodiments, the ComP protein comprises SEQ ID NO: 1 (ComP_{ADP1}: AAC45886.1), SEQ ID NO: 2 (ComP_{110264:} ENV58402.1), SEQ ID NO: 3 (ComP_{GFJ-2}: APV36638.1), SEQ ID NO: 4 (Com_{P50v1}: PKD82822.1), SEQ ID NO: 5 (ComP₄₄₆₆: SNX44537.1), or SEQ ID NO: 6 (ComP_{SFC}: OAL75955.1).

In certain embodiments, a ComP glycosylation tag of the current disclosure can be defined as comprising or consisting of the amino acid consensus sequence of SEQ ID NO: 27:

(SEQ ID NO: 27)
X₁X₂GTX₅X₆X₇X₈X₉X₁₀X₁₁X₁₂CX₁₄GVX₁₇X₁₈IX₂₀X₂₁X₂₂ASX₂₅X₂₆TX₂₈N
VX₃₁X₃₂AX₃₄CX₃₆X₃₇X₃₈X₃₉X₄₀X₄₁X₄₂X₄₃X₄₄

wherein: X₁ is V, A, or no amino acid;
X₂ is A, G, T, or no amino acid;
X₅ is P, S, or Q;
X₆ is S, M, or I;
X₇ is T, P, or V;
X₈ is A, S, or T;
X₉ is G, N, S, or T;
X₁₀ is N or no amino acid;
X₁₁ is S, G, or A;
X₁₂ is S or N;
X₁₄ is V, T, or A;
X₁₇ is Q, T, or E;
X₁₈ is E, Q, or T;
X₂₀ is S, N, A, or G;
X₂₁ is S or no amino acid;
X₂₂ is G or no amino acid;
X₂₅ is N, S, Or A;
X₂₆ is A, S, or K;
X₂₈ is T, S, or K;
X₃₁ is A or E;
X₃₂ is T or S;
X₃₄ is T, Q, or A;
X₃₆ is G, S, Or T;
X₃₇ is A, G, or D;
X₃₈ is S, L, or A;
X₃₉ is S, G, D, or T;
X₄₀ is A, V, or G;
X₄₁ is G, I, or V;
X₄₂ is Q, T, or I;
X₄₃ is I, V, T, or L; and
X₄₄ is I, T, or V.

In certain embodiments, a ComP glycosylation tag comprises or consists of a fragment of the amino acid consensus sequence of SEQ ID NO: 27, wherein the fragment retains the cysteine residue at position 13 of SEQ ID NO: 27, the cysteine residue at position 35 of SEQ ID NO: 27, and the serine residue at position 24 of SEQ ID NO: 27. In certain embodiments, a ComP glycosylation tag comprises or consists of a variant of the amino acid consensus sequence of SEQ ID NO: 27 or a fragment thereof, having one, two, three, four, five, six, or seven amino acid substitutions, additions, and/or deletions, however, wherein the variant maintains the cysteine residue at position 13 of SEQ ID NO: 27, the cysteine residue at position 35 of SEQ ID NO: 27, and the serine residue at position 24 of SEQ ID NO: 27. In certain embodiments, the amino acid substitution is a conservative amino acid substitution. As disclosed herein, in certain embodiments, a ComP glycosylation tag comprising SEQ ID NO: 27 does not comprise a methionine residue in a position corresponding to the conserved methionine residue at position 104 of SEQ ID NO: 2 (ComPiio264: ENV58402.1). Further, in certain embodiments, the amino acid sequence of a ComP glycosylation tag comprising SEQ ID NO: 27 does not extend in the C-terminus direction beyond the amino acid residue corresponding to position 44 of SEQ ID NO: 27. In certain embodiments, a ComP glycosylation tag comprising or consisting of the amino acid consensus sequence of SEQ ID NO: 27 or fragment and/or variant thereof is not more than 25, 30, 40, 45, or 50 amino acids in length.

In certain embodiments, a ComP glycosylation tag of the current disclosure can be defined as comprising or consisting of the amino acid consensus sequence of SEQ ID NO: 28:

(SEQ ID NO: 28)
CX₂GVX₅X₆IX₈X₉X₁₀ASX₁₃X₁₄TX₁₆NVX₁₉X₂₀AX₂₂C

wherein:
X₂ is V, T, or A, optionally V;
X₅ is Q, T, or E, optionally Q;
X₆ is E, Q, or T;
X₈ is S, N, A, or G;
X₉ is S or no amino acid;
X₁₀ is G or no amino acid;
X₁₃ is N, S, or A, optionally N;
X₁₄ is A, S, or K, optionally A;
X₁₆ is T, S, or K;
X₁₉ is A or E, optionally A;
X₂₀ is T or S, optionally T; or
X₂₂ is T, Q, or A, optionally T.

In certain embodiments, a ComP glycosylation tag comprises or consists of a variant of the amino acid consensus sequence of SEQ ID NO: 28 having one, two, three, four, five, six, or seven amino acid substitutions, additions, and/or deletions, however, wherein the variant maintains the cysteine residue at position 1 of SEQ ID NO: 28, the cysteine residue at position 23 of SEQ ID NO: 28, and the serine residue at position 12 of SEQ ID NO: 28. In certain embodiments, the amino acid substitution is a conservative amino acid substitution.

In certain embodiments, a ComP glycosylation tag comprising SEQ ID NO: 28 does not comprise a methionine residue in a position corresponding to the conserved methionine residue at position 104 of SEQ ID NO: 2 (ComPiio264: ENV58402.1). Further, in certain embodiments, the amino acid sequence of a ComP glycosylation tag comprising SEQ ID NO: 28 does not extend in the C-terminus direction beyond the amino acid residue corresponding to position 103 of SEQ ID NO: 2 (ComP₁₁₀₂₆₄: ENV58402.1). In certain embodiments, a ComP glycosylation tag comprising the amino acid consensus sequence of SEQ ID NO: 28 or variant thereof is not more than 25, 30, 40, 45, or 50 amino acids in length.

In certain embodiments, the ComP glycosylation tag comprises or consists of a variant thereof having one, two, three, four, five, six, or seven amino acid substitutions, additions, and/or deletions of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2]; SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO: 58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7]; SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11], wherein the variant maintains both a cysteine residue corresponding to the conserved cysteine residue at position 75 of SEQ ID NO: 1 (ComP_{ADP1}: AAC45886.1) and a cysteine residue corresponding to the conserved cysteine residue at position 95 of SEQ ID NO: 1 and the variant maintains a serine residue corresponding to the conserved serine residue at position 84 of SEQ ID NO: 1. In certain embodiments, the amino acid substitution is a conservative amino acid substitution. Further, in certain embodiments, the ComP glycosylation tag comprises or consists of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2]; SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO: 58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7]; SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11]. In certain embodiments, such a ComP glycosylation tag comprising one of the above sequences or variants thereof does not comprise a methionine residue in a position corresponding to the conserved methionine residue at position 104 of SEQ ID NO: 2 (ComP₁₁₀₂₆₄: ENV58402.1). Further, in certain embodiments, the amino acid sequence of such a ComP glycosylation tag comprising one of the above sequences or variants thereof does not extend in the C-terminus direction beyond the amino acid residue corresponding to position 103 of SEQ ID NO: 2 (ComP₁₁₀₂₆₄: ENV58402.1). In certain embodiments, a ComP glycosylation tag comprising an amino acid sequence and/or variant thereof listed above is not more than 25, 30, 40, 45, or 50 amino acids in length. In certain embodiments, a ComP glycosylation tag consists of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2]; SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO: 58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7]; SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11].

In certain embodiments, the oligo- or polysaccharide for conjugation to the glycosylation tag, fusion protein, and/or bioconjugate is produced by a bacteria from the genus Streptococcus. For example, in certain embodiments, the polysaccharide is a S. pneumoniae, S. agalactiae, or S. suis capsular polysaccharide. Further, in certain embodiments, the capsular polysaccharide is CPS14, CPS8, CPS9V, or CPS15b. In certain other embodiments, the oligo- or polysaccharide is produced by a bacteria from the genus Klebsiella. For example, in certain embodiments, the polysaccharide is a Klebsiella pneumoniae, Klebsiella varricola, Klebsiella michinganenis, or Klebsiella oxytoca capsular polysaccharide. In certain embodiments, the polysaccharide is a Klebsiella pneumoniae capsular polysaccharide. Further, in certain embodiments, the polysaccharide is a serotype K1 or serotype K2 capsular polysaccharide of Klebsiella pneumoniae.

In certain embodiments, the bioconjugate is produced in vivo. For example, in certain embodiments, the bioconjugate is produced in a bacterial cell.

As the bioconjugate comprises an oligo- or polysaccharide covalently linked to a fusion protein, in certain applications, it may be advantageous to form a fusion protein with a carrier protein or fragment thereof In certain embodiments, the carrier protein is one recognized in the art as useful in producing conjugate vaccines. In certain embodiments, when a ComP glycosylation tag fragment is fused to a carrier protein or fragment thereof, the glycosylation tag fragment and thus the fusion protein, can be glycosylated at the conserved serine residue described elsewhere herein. In certain embodiments, the fusion protein comprises a carrier protein selected from the group consisting of diphtheria toxoid CRM₁₉₇, tetanus toxoid, Pseudomonas aeruginosa Exotoxin A (EPA), tetanus toxin C fragment, cholera toxin B subunit, Haemophilus influenza protein D, or a fragment thereof In certain embodiments, the carrier protein or fragment thereof is linked to the ComP glycosylation tag via an amino acid linker, for example (GGGS). (SEQ ID NO: 23), wherein n is at least one or AAA (SEQ ID NO: 24). In order to increase the potential immunogenicity of a ComP fusion protein, it may be advantageous to include more than one glycosylation tag. Thus, in certain embodiments, the fusion protein comprise two or more, three or more, four or more, five or more, six or more, eight or more, ten or more, fifteen or more, or twenty or more ComP glycosylation tags. In certain embodiments, the fusion protein comprises any of 2, 3, 4, 5, 6, 7, 8, 9, 10, 11, 12, 13, 14, 15, or 20 to any of 3, 4, 5, 6, 7, 8, 9, 10, 11, 12, 13, 14, 15, 20, or 25 ComP glycosylation tags. In certain embodiments, multiple glycosylation tags are arranged in tandem to one another in the fusion protein. In certain embodiments, multiple glycosylation tags are arranged apart from one another in the fusion protein, for example separated by sequences of carrier protein. In certain embodiments, the glycosylation tag(s) can be, for example, located at the N-terminal end of the carrier protein and/or fusion protein. In certain embodiments, the glycosylation tag(s) can be, for example, located at the C-terminal end of the carrier protein and/or fusion protein. In certain embodiments, the glycosylation tag(s) can be located internally within the carrier protein and/or fusions protein, for example, wherein a glycosylation tag is located between multiple carrier proteins in a fusion protein. In certain embodiments, the multiple carrier proteins can be identical in type or different in type. In certain embodiments, the glycosylation tags can be identical in type or different in type. In certain embodiments, these ComP glycosylation tags are identical. In certain embodiments, at least two of the ComP glycosylation tags differ from each other. In certain embodiments, at least three, at least four, or at least five of the ComP glycosylation tags all differ from each other. Further, in certain embodiments, none of the ComP glycosylation tags are the same.

A bioconjugate of this invention may have one of numerous uses including, but not limited to, use as a conjugate vaccine. For example, in certain embodiments, the conjugate vaccine is a vaccine against Streptococcus pneumoniae serotype 8, Streptococcus pneumoniae serotype 1, Streptococcus pneumoniae serotype 2, Streptococcus pneumoniae serotype 4, Streptococcus pneumoniae serotype 5, Streptococcus pneumoniae serotype 6A, Streptococcus pneumoniae serotype 6B, Streptococcus pneumoniae serotype 7F, Streptococcus pneumoniae serotype 9N, Streptococcus pneumoniae serotype 9V, Streptococcus pneumoniae serotype 10A, Streptococcus pneumoniae serotype 11A, Streptococcus pneumoniae serotype 12F, Streptococcus pneumoniae serotype 14, Streptococcus pneumoniae serotype 15B, Streptococcus pneumoniae serotype 17F, Streptococcus pneumoniae serotype 18C, Streptococcus pneumoniae serotype 19F, Streptococcus pneumoniae serotype 19A, Streptococcus pneumoniae serotype 20, Streptococcus pneumoniae serotype 22F, Streptococcus pneumoniae serotype 23F, Streptococcus pneumoniae serotype 33F, Klebsiella pneumoniae serotype K1, Klebsiella pneumoniae serotype K2, Klebsiella pneumoniae serotype K5, Klebsiella pneumoniae serotype K16, Klebsiella pneumoniae serotype K20, Klebsiella pneumoniae serotype K54, Klebsiella pneumoniae serotype K57, Streptococcus agalactiae serotype Ia, Streptococcus agalactiae serotype Ib, Streptococcus agalactiae serotype II, Streptococcus agalactiae serotype III, Streptococcus agalactiae serotype IV, Streptococcus agalactiae serotype V, Streptococcus agalactiae serotype VI, Streptococcus agalactiae serotype VII, Streptococcus agalactiae serotype VIII, Streptococcus agalactiae serotype IX, Streptococcus pyogenes Group A Carbohydrate, Enterococcus faecalis serotype A, Enterococcus faecalis serotype B, Enterococcus faecalis serotype C, Enterococcus faecalis serotype D, Enterococcus faecium capsular polysaccharide and lipotechoic acid, Moraxella catarrhalis lipooligosaccharide A, Moraxella catarrhalis lipooligosaccharide B, Moraxella catarrhalis lipooligosaccharide C, and Staphylococcus aureus lipotechoic acid.

In certain embodiments, the conjugate vaccine is useful because it induces an immune response when administered to a subject. In certain embodiments, the immune response elicits long term memory (memory B and T cells), is an antibody response, and is optionally a serotype-specific antibody response. In certain embodiments, the antibody response is an IgG or IgM response. For example, in certain embodiments the antibody response can be an IgG response, and in certain embodiments, an IgG1 response. In certain embodiments, the conjugate vaccine generates immunological memory in a subject administered the vaccine.

Provided for herein is a fusion protein as disclosed in further detail elsewhere herein and comprising a ComP glycosylation tag as disclosed in detail elsewhere herein. In certain embodiments, the fusion protein is glycosylated at a serine residue on the glycosylation tag corresponding to the serine residue at position 84 of SEQ ID NO: 1 (ComP_{ADP1}: AAC45886.1). In certain embodiments, the fusion protein is glycosylated with an oligo- or polysaccharide. In certain embodiments, the oligo- or polysaccharide is produced by a bacteria from the genus Streptococcus such as, for example, a S. pneumoniae, S. agalactiae, or S. suis capsular polysaccharide. In certain embodiments, the capsular polysaccharide is CPS14, CPS8, CPS9V, or CPS15b. In certain embodiments, the oligo- or polysaccharide is produced by a bacteria from the genus Klebsiella, for example, a Klebsiella pneumoniae, Klebsiella varricola, Klebsiella michinganenis, or Klebsiella oxytoca capsular polysaccharide. In certain embodiments, the polysaccharide is a Klebsiella pneumoniae capsular polysaccharide. In certain embodiments, the polysaccharide is a serotype K1 or serotype K2 capsular polysaccharide of Klebsiella pneumoniae. In certain of any embodiments disclosed herein, the oligo- or polysaccharide comprises a glucose at its reducing end. Certain embodiments are drawn a fusion protein wherein the fusion protein is produced in vivo. For example, in certain embodiments, the fusion protein is produced in a mammalian cell, fungal cell, yeast cell, insect cell, avian cell, algal cell, or bacterial cell. In certain embodiments, the fusion protein is produced in a bacterial cell, for example, E. coli.

Disclosed herein are methods for the in vivo conjugation of an oligo- or polysaccharide to a polypeptide (in vivo glycosylation). In certain embodiments, the method comprises covalently linking the oligo- or polysaccharide to the polypeptide with a PglS oligosaccharyltransferase (OTase) (described elsewhere herein). In certain embodiments, the polypeptide comprises a ComP protein or a glycosylation tag thereof In certain embodiments, the polypeptide comprises a ComP protein or a glycosylation tag thereof linked to a heterologous polypeptide such as a carrier protein. Representative examples of PglS OTases include, but are not limited to PglS_{110264,} PglS_{ADP1}, PglS_{GFJ-2}, PglS₅₀ᵥ₁, PglS_{4466,} and PglS_{SFC}. ComP proteins are described in detail elsewhere and representative examples include, but are not limited to ComP₁₁₀₂₆₄, COMP_{ADP1}, COMP_{GFJ-2}, COMP₅₀ᵥ₁, ComP₄₄₆₆, and ComP_{SFC}. It will be recognized that while a PglS OTase from an organism would naturally glycosylate the ComP protein from that organism (e.g., PglS₁₁₀₂₆₄ glycosylates ComP₁₁₀₂₆₄) in certain embodiments, a PglS from one organism glycosylates a ComP from a different organism (e.g., PglS_{ADP1} glycosylates ComP₁₁₀₂₆₄). For example, in certain aspects, the PglS OTase is PglS_{ADP1}. In certain embodiments, where the PglS OTase is PglS_{ADP1}, the ComP protein glycosylated is not ComP_{ADP1}. For example, in certain embodiments where the PglS OTase is PglS_{ADP1}, the ComP protein is ComP_{1100264.} Of course, it will be recognized that a PglS OTase does not naturally glycosylate a ComP protein or a glycosylation tag fragment thereof, even from the same organism as the PglS Otase, when the ComP protein or glycosylation tag fragment thereof is linked to a heterologous carrier protein.

In certain embodiments for any combination of PglS and ComP, the ComP protein or glycosylation tag fragment thereof is glycosylated at a serine residue corresponding to the serine residue at position 84 of SEQ ID NO: 1 (ComP_{ADP1}: AAC45886.1).

In certain embodiments disclosed herein, the in vivo glycosylation occurs in a host cell. In certain embodiments, for example, the host cell can be a mammalian cell, fungal cell, yeast cell, insect cell, avian cell, algal cell, or bacterial cell. In certain embodiments, the host cell is a bacterial cell, for example, E. coli.

In certain embodiments, the method comprises culturing a host cell comprising the components necessary for the conjugation of the oligo- or polysaccharide to the polypeptide. In general, these components are the oligosaccharyltransferase, the acceptor polypeptide to be glycosylated, and the oligo- or polysaccharide. In certain embodiments, the method comprises culturing a host cell that comprises: (a) a genetic cluster encoding for the proteins required to synthesize the oligo- or polysaccharide; (b) a PglS OTase; and (3) the acceptor polypeptide. Further, it has been discovered that production of the oligo- or polysaccharide can be enhanced by a transcriptional activator. In certain embodiments, the production of the oligo- or polysaccharide is enhanced by the K. pneumoniae transcriptional activator rmpA (K. pneumoniae NTUH K-2044) or a homolog of the K. pneumoniae transcriptional activator rmpA (K. pneumoniae NTUH K-2044). In certain embodiments, the method further comprises expressing and/or providing such a transcriptional activator in the host cell along with the other components.

In certain embodiments, the carrier protein linked to the ComP glycosylation tag is, for example, diphtheria toxoid CRM₁₉₇, tetanus toxoid, Pseudomonas aeruginosa Exotoxin A (EPA), tetanus toxin C fragment, cholera toxin B subunit, Haemophilus influenza protein D, or a fragment thereof.

Certain embodiments also provide for a host cell comprising the components for in vivo glycosylation of an acceptor ComP protein or glycosylation tag fragment thereof In certain embodiments, a host cell comprises: (a) a genetic cluster encoding for the proteins required to synthesize an oligo- or polysaccharide; (b) a PglS OTase; and (3) an acceptor polypeptide comprising a ComP protein or a glycosylation tag fragment thereof. In certain embodiments, the acceptor polypeptide is a fusion protein. In certain embodiments, the host cell further comprises a transcriptional activator such as described above along with the other components.

In certain embodiments, a host cell comprises an isolated nucleic acid encoding a PglS OTase. In certain embodiments a host cell comprises an isolated nucleic acid encoding the ComP acceptor polypeptide. In certain embodiments, a host cell comprises a genetic cluster encoding for the proteins required to synthesize an oligo- or polysaccharide. In certain embodiments, a host cell comprises at least two of an isolated nucleic acid encoding a PglS OTase, an isolated nucleic acid encoding the ComP acceptor polypeptide, and genetic cluster encoding for the proteins required to synthesize an oligo- or polysaccharide. In embodiments aspects, a host cell comprises a nucleic acid encoding a PglS OTase of one organism and a nucleic acid encoding the ComP acceptor polypeptide from a different organism.

Certain embodiments also provide for an isolated nucleic acid encoding the ComP protein, ComP glycosylation tag fragment, and/or ComP fusion protein described anywhere herein. In certain embodiments, an isolated nucleic acid referred to herein is a vector or is contained within a vector. In certain embodiments, an isolated nucleic acid referred to herein is inserted and/or has been incorporated into a heterologous genome or a heterologous region of a genome.

It is contemplated that a conjugate vaccine (such as the EPA or MrkA vaccine construct) can comprise additional/multiple sites of glycosylation to increase the glycan to protein ratio as well as expand upon the number of serotypes in order to develop a comprehensive bioconjugate vaccine.

In certain embodiments, a bioconjugate or glycosylated fusion protein disclosed herein is a conjugate vaccine that can be administered to a subject for the prevention and/or treatment of an infection and/or disease. In certain embodiments, the conjugate vaccine is a prophylaxis that can be used, e.g., to immunize a subject against an infection and/or disease. In certain embodiments, the bioconjugate is associated with (such as in a therapeutic composition) and/or administered with an adjuvant. Certain embodiments provide for a composition (such as a therapeutic composition) comprising a conjugate vaccine described herein and an adjuvant. In certain embodiments, when the conjugate vaccine is administered to a subject, it induces an immune response. In certain embodiments, the immune response elicits long term memory (memory B and T cells). In certain embodiments, the immune is an antibody response. In certain embodiments, the antibody response is a serotype-specific antibody response. In certain embodiments, the antibody response is an IgG or IgM response. In certain embodiments where the antibody response is an IgG response, the IgG response is an IgG1 response. Further, in certain embodiments, the conjugate vaccine generates immunological memory in a subject administered the vaccine.

Certain embodiments also provide for producing a vaccine against an infection and/or disease. In certain embodiments a method comprises isolating a bioconjugate or fusion protein disclosed herein (conjugate vaccine) and combining the conjugate vaccine with an adjuvant. In certain embodiments, the infection is a localized or systemic infection of skin, soft tissue, blood, or an organ, or is auto-immune in nature. In certain embodiments, the vaccine is a conjugate vaccine against infection. In certain embodiments, the disease is pneumonia. In certain embodiments, the infection is a systemic infection and/or an infection of the blood. In certain embodiments, the subject is a mammal. For example, in certain embodiments, a pig or a human.

Provided herein are methods of inducing a host immune response against a pathogen. In certain embodiments, the pathogen is a bacterial pathogen. In certain embodiments, the host is immunized against the pathogen. In certain embodiments, the method comprises administering to a subject in need of the immune response an effective amount of a ComP conjugate vaccine, glycosylated fusion protein, or any other therapeutic/immunogenic composition disclosed herein. Certain embodiments provide a conjugate vaccine, glycosylated fusion protein, or other therapeutic/immunogenic composition disclosed herein for use in inducing a host immune response against a bacterial pathogen and immunization against the bacterial pathogen. Examples of immune responses include but are not limited to an innate response, an adaptive response, a humoral response, an antibody response, cell mediated response, a B cell response, a T cell response, cytokine upregulation or downregulation, immune system cross-talk, and a combination of two or more of said immune responses. In certain embodiments, the immune response is an antibody response. In certain embodiments, the immune response is an innate response, a humoral response, an antibody response, a T cell response, or a combination of two or more of said immune responses.

Also provided herein are methods of preventing or treating a bacterial disease and/or infection in a subject comprising administering to a subject in need thereof a conjugate vaccine composition, a fusion protein, or a composition disclosed herein. In certain embodiments, the infection is a localized or systemic infection of skin, soft tissue, blood, or an organ, or is auto-immune in nature. In certain embodiments, the disease is pneumonia. In certain embodiments, the infection is a systemic infection and/or an infection of the blood. In certain embodiments disclosed herein, the subject is a vertebrate. In certain embodiments the subject is a mammal such as a dog, cat, cow, horse, pig, mouse, rat, rabbit, sheep, goat, guinea pig, monkey, ape, etc. And, for example, in certain embodiments the mammal is a human.

In any of the embodiments of administration disclose herein, the composition is administered via intramuscular injection, intradermal injection, intraperitoneal injection, subcutaneous injection, intravenous injection, oral administration, mucosal administration, intranasal administration, or pulmonary administration.

### EXAMPLES

### Example 1.

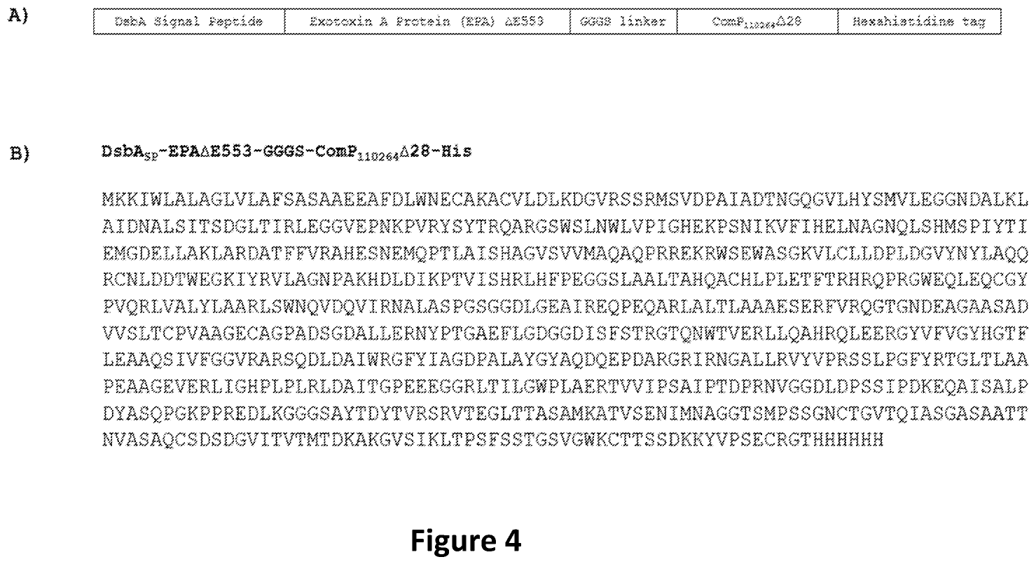

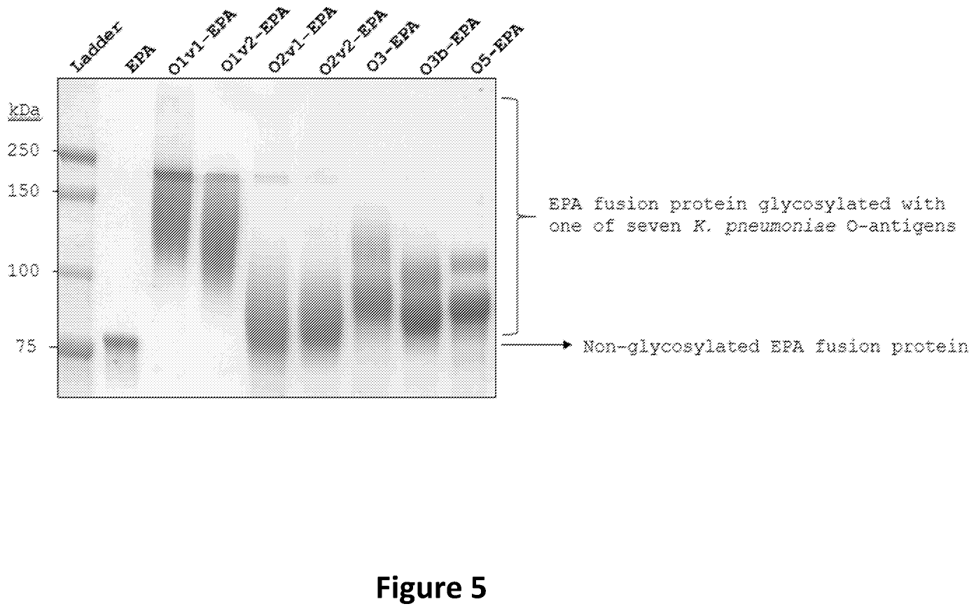

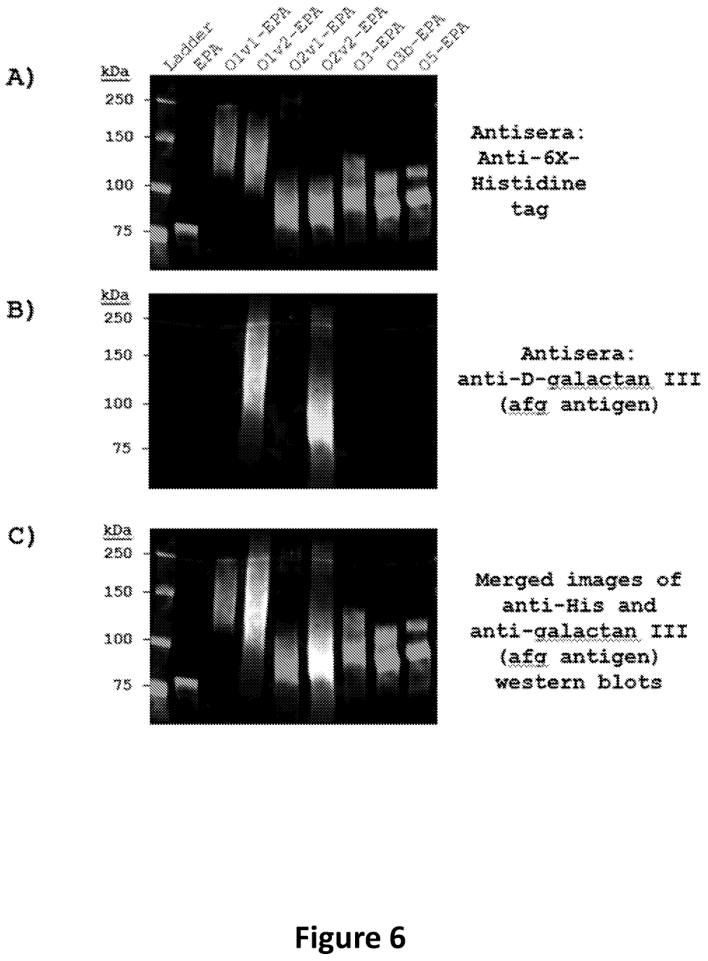

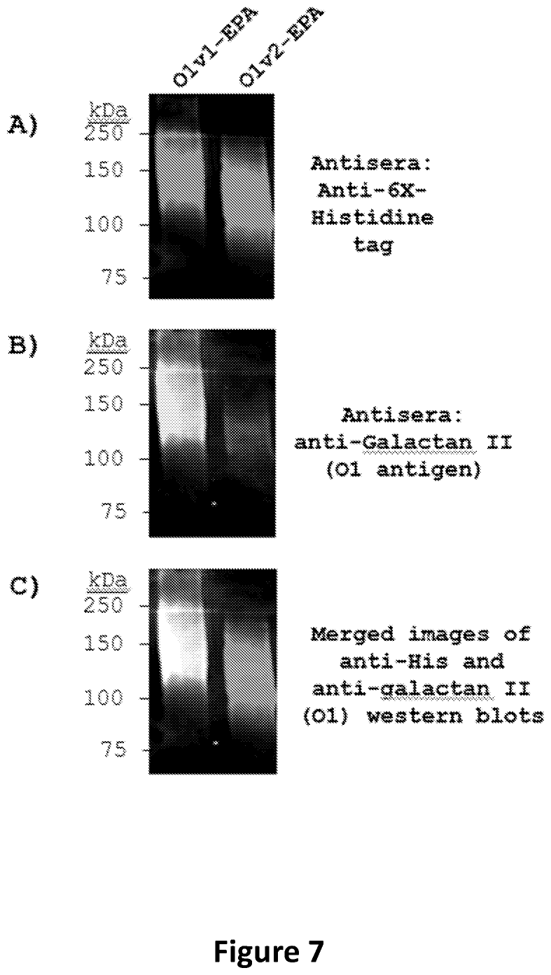

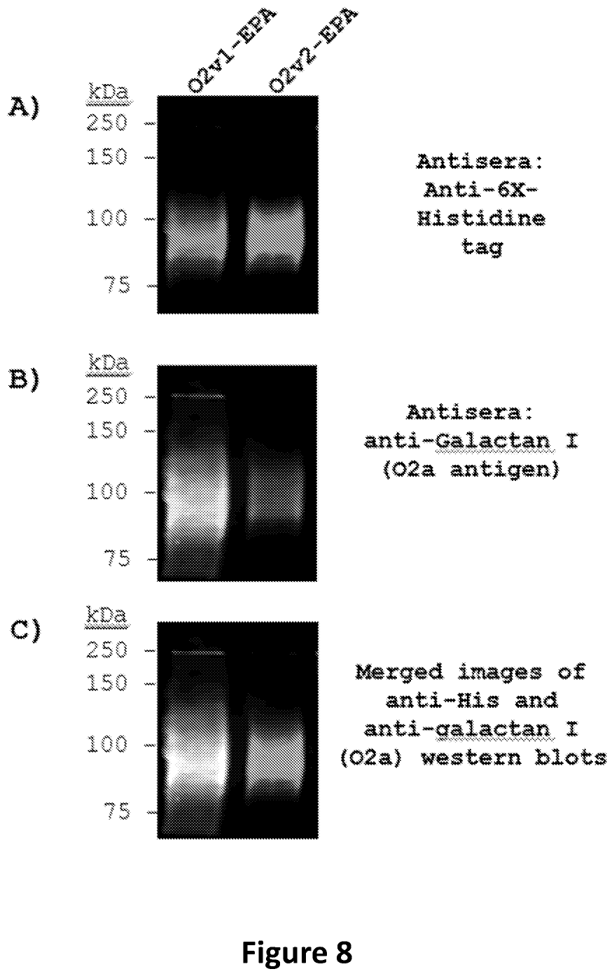

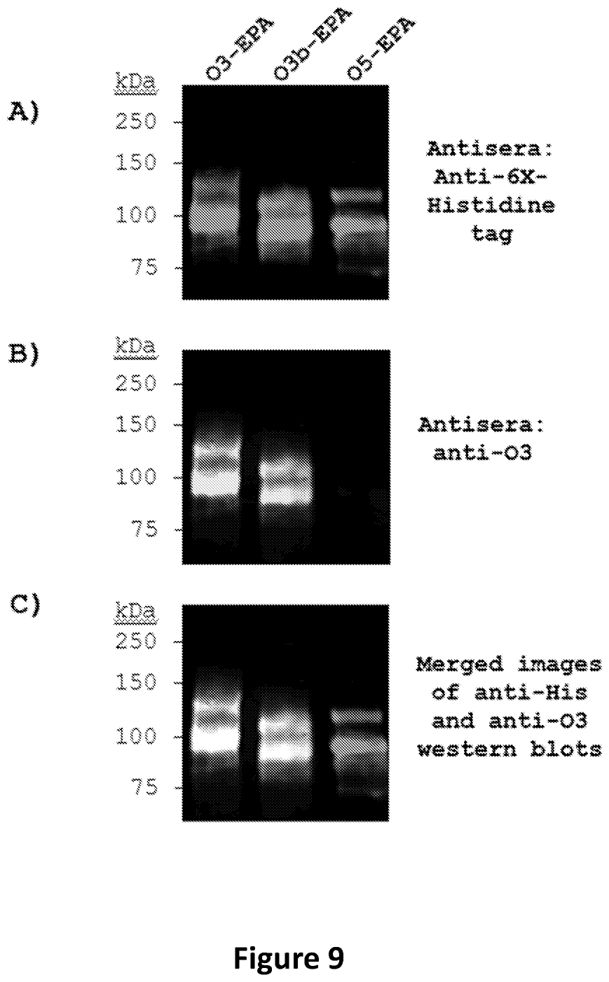

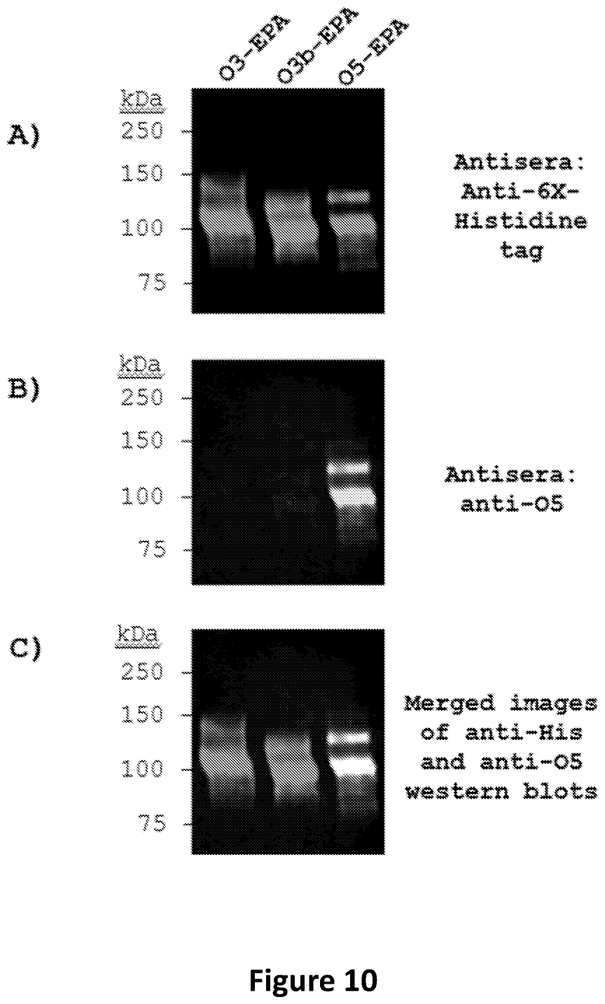

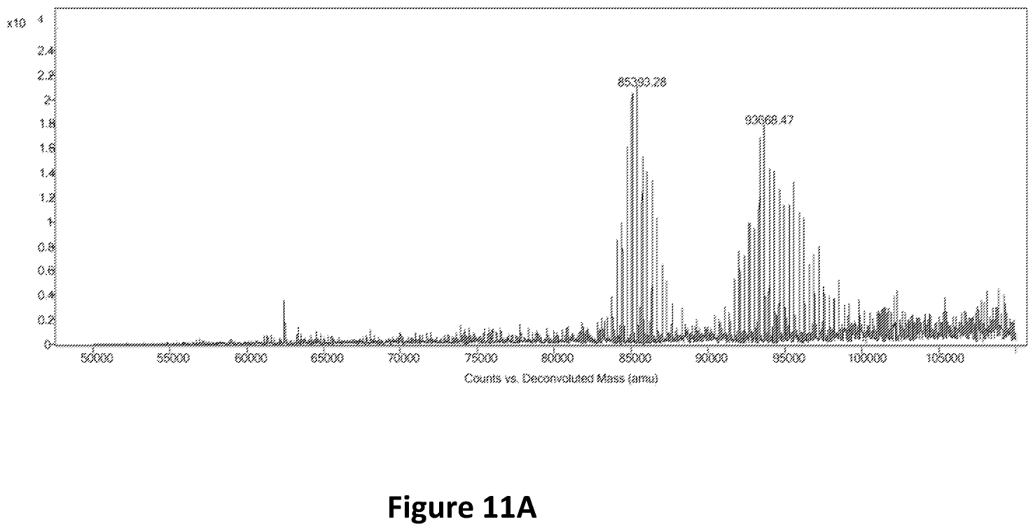

Modified exotoxin A protein of Pseudomonas aeruginosa carrier proteins can be glycosylated with K. pneumoniae O-antigen polysaccharides by PglS_{ADP1}. The inventors have developed methods for producing glycosylated proteins recombinantly in E. coli by combining the oligosaccharyltransferase PglS with a heterologously expressed polysaccharide as well as a modified carrier protein containing an O-linked glycosylation recognition motif. These methods utilize PglS from Acinetobacter baylyi ADP (PglS_{ADP1}), or an orthologous PglS variant, to transfer virtually any polysaccharide from a lipid-liked precursor to a modified carrier protein. FIG. 4 shows one such modified carrier protein, the genetically deactivated variant of exotoxin A protein (EPA) from Pseudomonas aeruginosa fused to a fragment of ComP (the natural acceptor protein of PglS) as the carrier protein (Harding, C. M. & Feldman, M. F., 2019; Feldman, M. F. et al., 2019). To maximize bioconjugate vaccine production, we use a ΔwααL mutant of E. coli (Faridmoayer, A., Fentabil, M. A., Mills, D. C., Klassen, J. S. & Feldman, M. F., 2007). The WaaL ligase is responsible for transferring the O-antigen to the core saccharide of Lipid-A thereby making lipopolysaccharide (Feldman, M. F. et al., 2005). Thus, strains lacking the WaaL ligase accumulate lipid-linked O-antigen precursors that the conjugating enzyme PglS_{ADP1} can then transfer to modified EPA carrier protein. When a plasmid expressing the modified EPA carrier protein is combined with a plasmid expressing PglS_{ADP1} and a plasmid expressing one of the seven K. pneumoniae O-antigens (O1v1, O1v2, O2v1, O2v2, O3, O3b, or O5) in the CLM24 strain of E. coli, we observe that the modified EPA carrier protein is robustly glycosylated with each of the seven O-antigen subtypes. FIG. 5 shows Coomassie stained SDS-PAGE analysis of K. pneumoniae O-antigen glycosylated modified EPA carrier proteins that were purified with nickel affinity chromatography, anionic exchange chromatography and then size exclusion chromatography. 5 μg of total protein for each sample was resolved via SDS-PAGE and stained with Gel-code blue Coomassie stain. As seen in FIG. 5, the non-glycosylated modified EPA carrier protein migrates slightly above the 75 kDa marker (theoretical molecular weight 79,526.15 Daltons). The O1v1-EPA and O1v2 -EPA bioconjugates are observed as largest by size migrating on the SDS-PAGE gel between 100-250 kDa. The O2v1-EPA and O2v2-EPA bioconjugates are the smallest, which correlates with the previously published sizes of the O1 and O2 LPS preparations (Clarke, B. R. et al., 2018). As seen in FIG. 6, western blotting with antisera specific to the D-galactan-III epitope exclusively reacted with the O1v2-EPA and O2v2 -EPA samples demonstrating their unique serological reactivity. In addition, western blotting was performed for each serogroup (O1, O2, O3, and O5) containing EPA bioconjugates confirming the correct immunological reactivity. FIG. 7 shows western blotting of the O1v1-EPA and the O1v2-EPA modified carrier proteins probed with anti-His antisera and antisera specific to the D-galactan II epitope. FIG. 8 shows western blotting of the O2v1-EPA and the O2v2-EPA modified carrier proteins probed with anti-His antisera and antisera specific to the D-galactan I epitope. FIG. 9 shows western blotting of the O3-EPA, the O3b-EPA and the O5-EPA modified carrier proteins probed with anti-His antisera and antisera specific to that recognizes the O3 antigen of K. pneumoniae. The polyclonal O3 antisera is not able to discriminate between the O3 and O3b serotypes, which is observed for many pneumococcal CPS serotypes that are also closely related, i.e. polyclonal serotype cannot distinguish between serotype 19A and 19F or 9V and 9N. FIG. 10 shows western blotting of the O3-EPA, the O3b-EPA and the O5-EPA modified carrier proteins probed with anti-His antisera and antisera specific to that recognizes the O5 antigen of K. pneumoniae. Anti-glycan antisera for the D-galactan II (also known as O1 or O1v1) (McCallum, K. L., Schoenhals, G., Laakso, D., Clarke, B. & Whitfield, C., 1989), D-galactan I (also known as O2a or O2v1) (Clarke, B. R. et al., 2018), and D-galactan III (also known as O2afg or O2v2 ) (Clarke, B. R. et al., 2018) antigens were previously authenticated. The K. pneumoniae O3 and O5 antigens are identical to the O9 and O8 antigens of E. coli, respectively (Greenfield, L. K. et al., 2012; Saeki, A. et al., 1993). As such, commercially available anti-sera against the E. coli O8 and O9 antigens from Statens Serum Institut were used to probe for the O3 and O5 K. pneumoniae serogroups in.

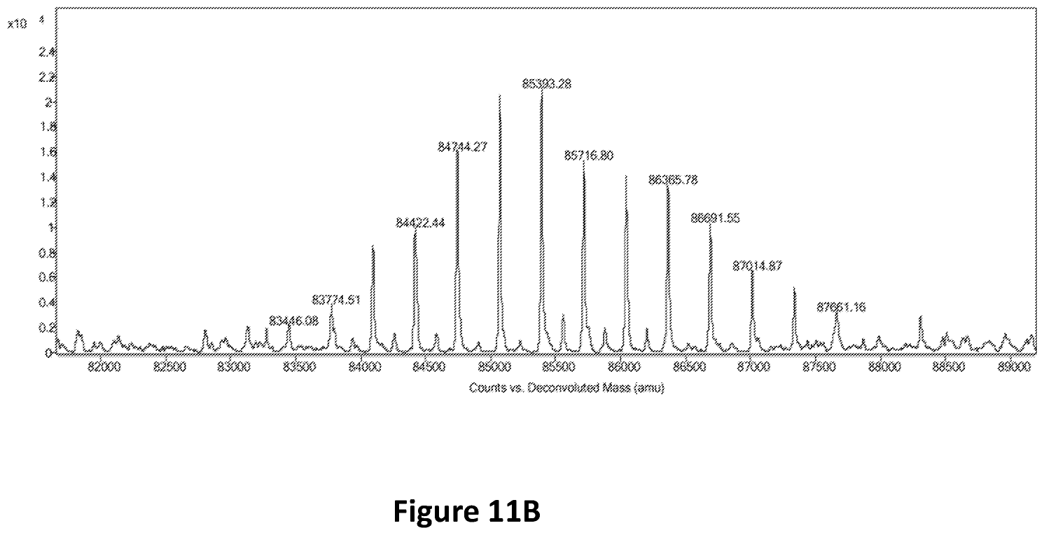

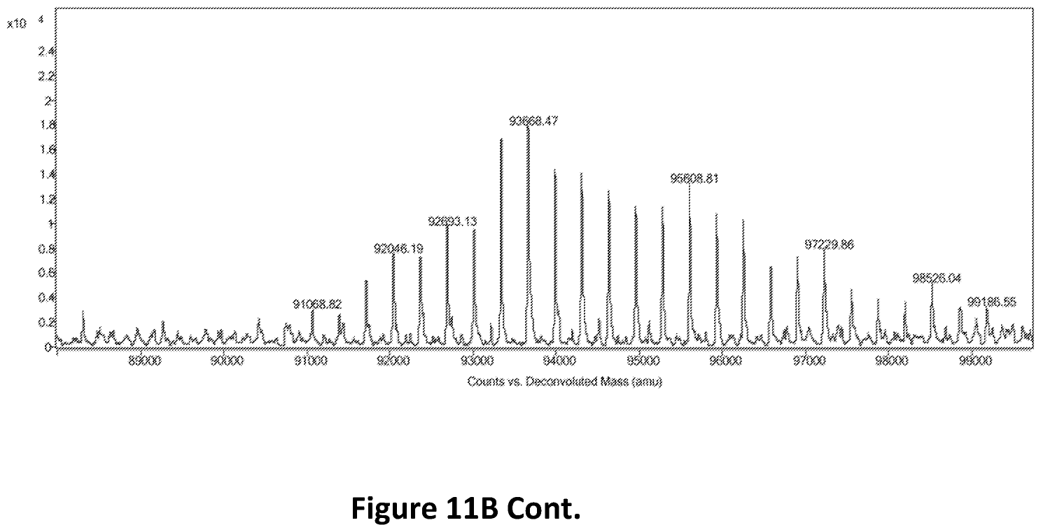

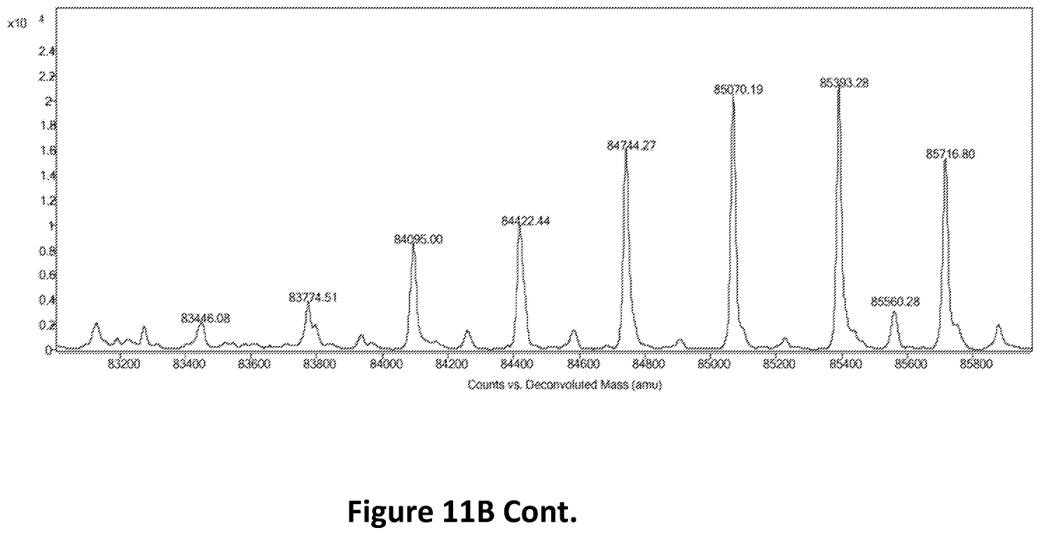

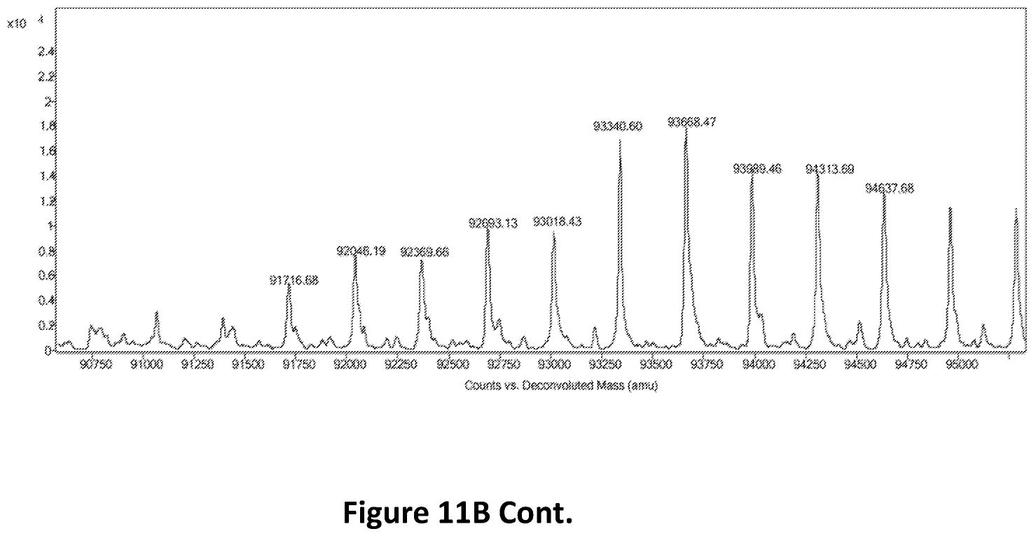

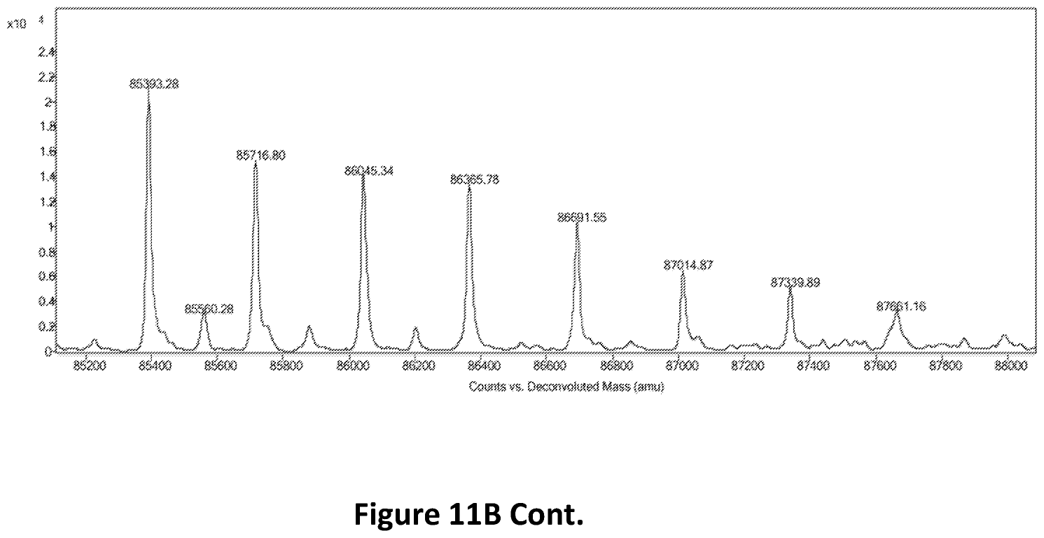

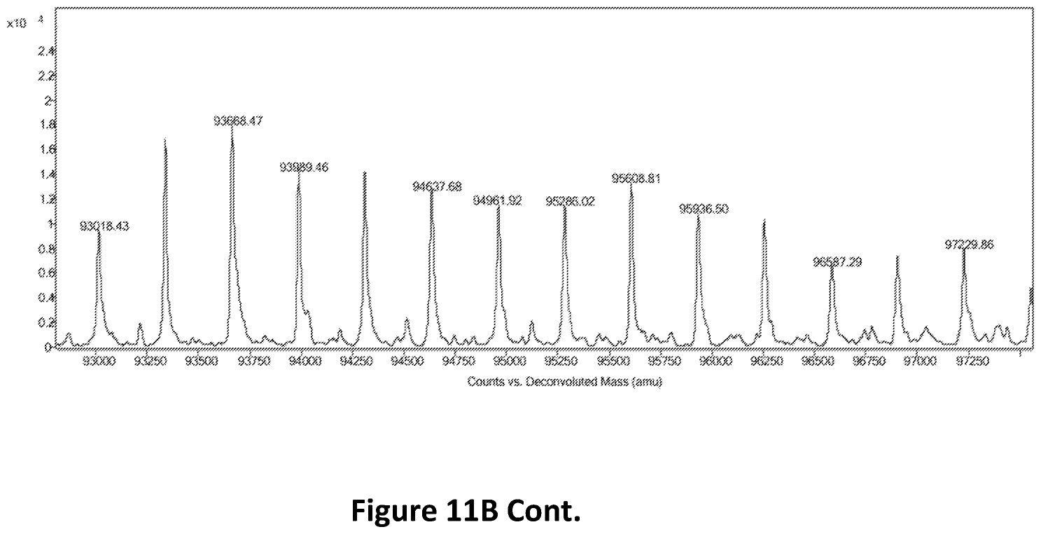

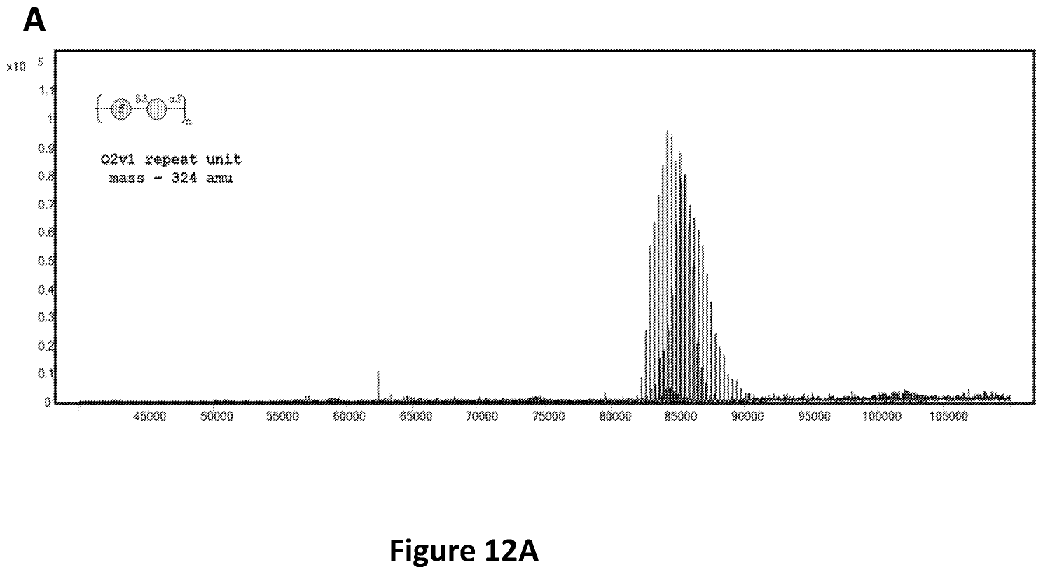

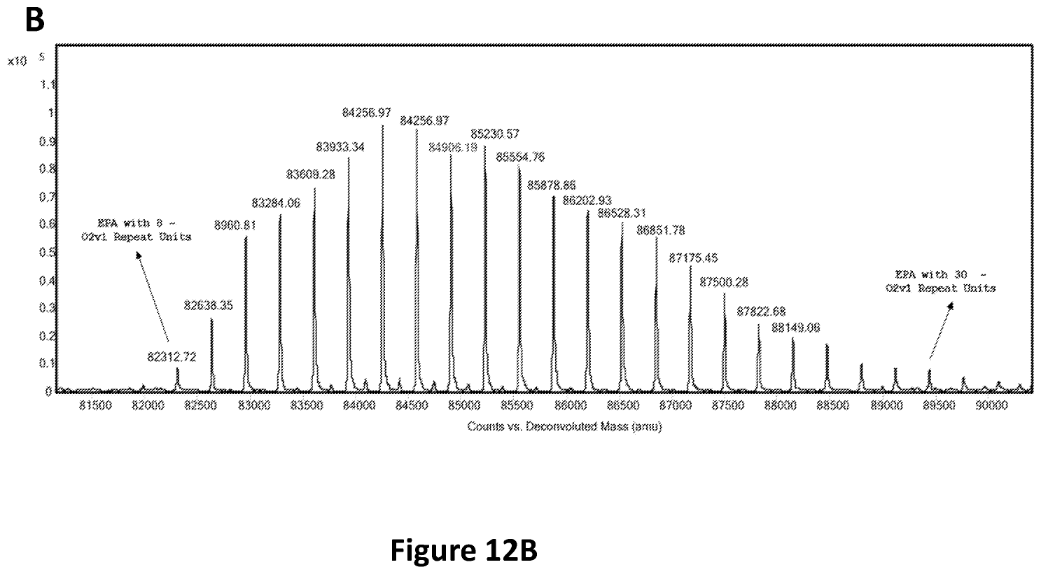

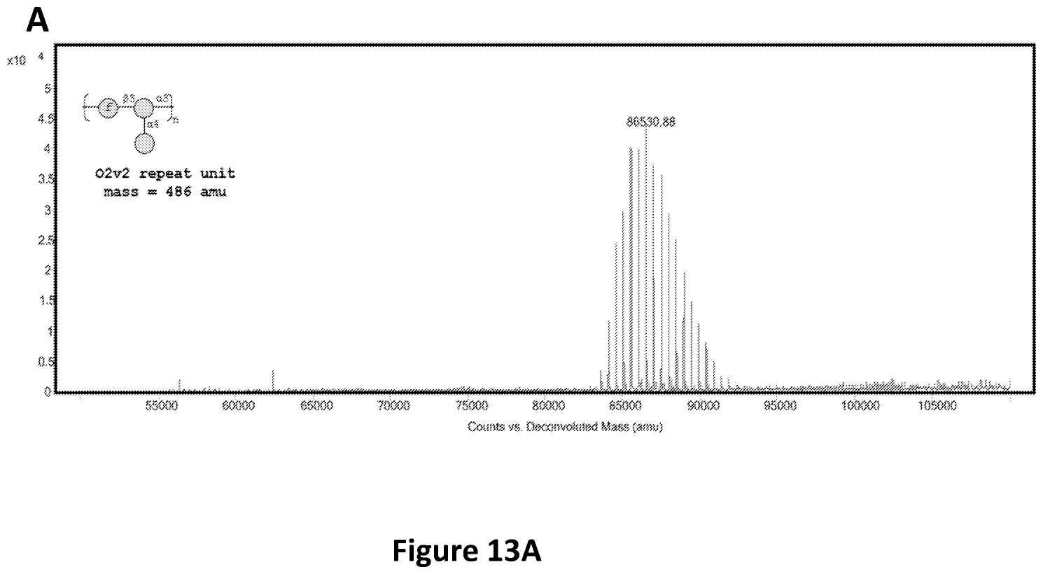

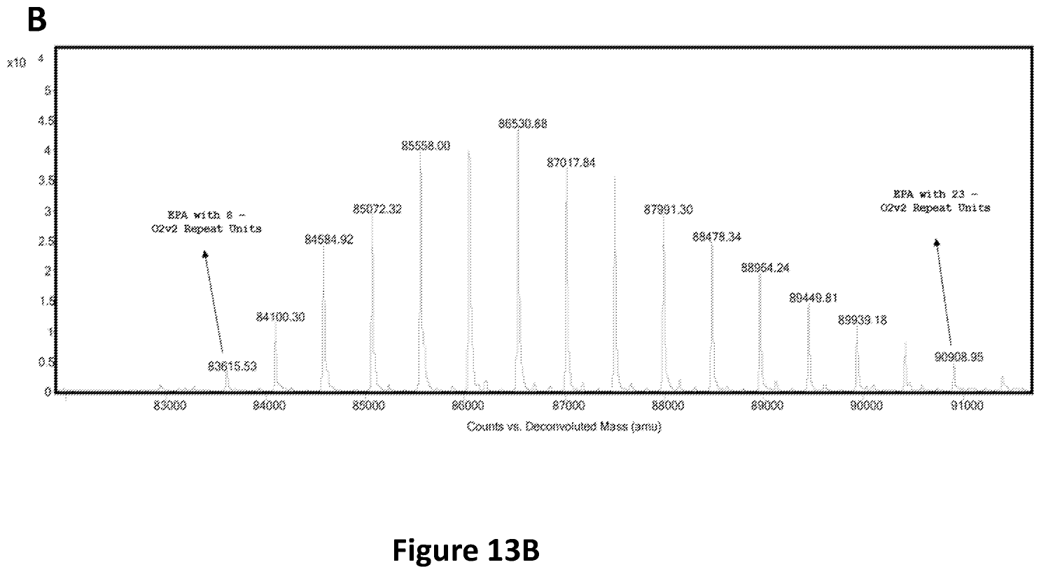

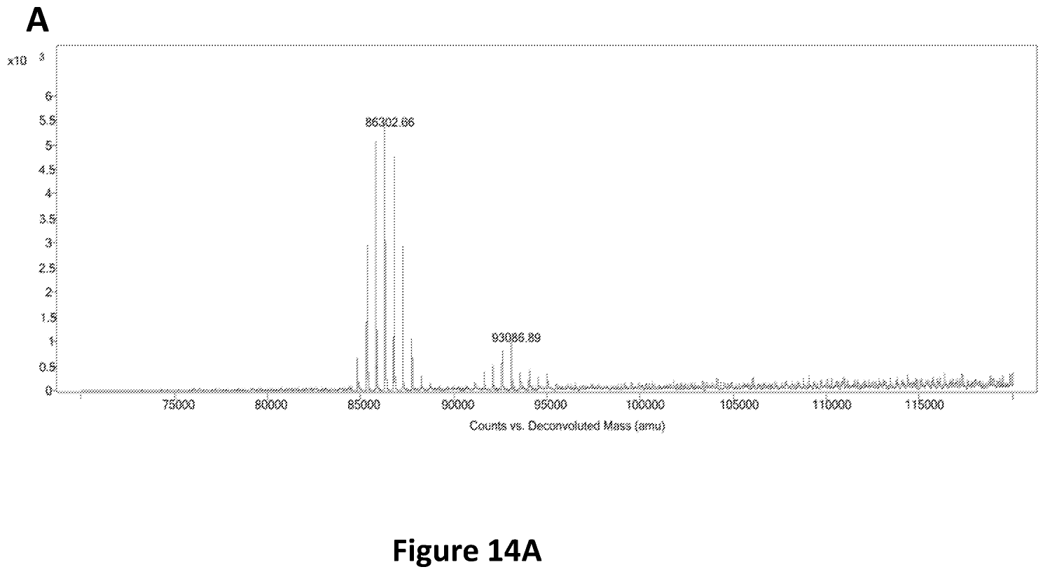

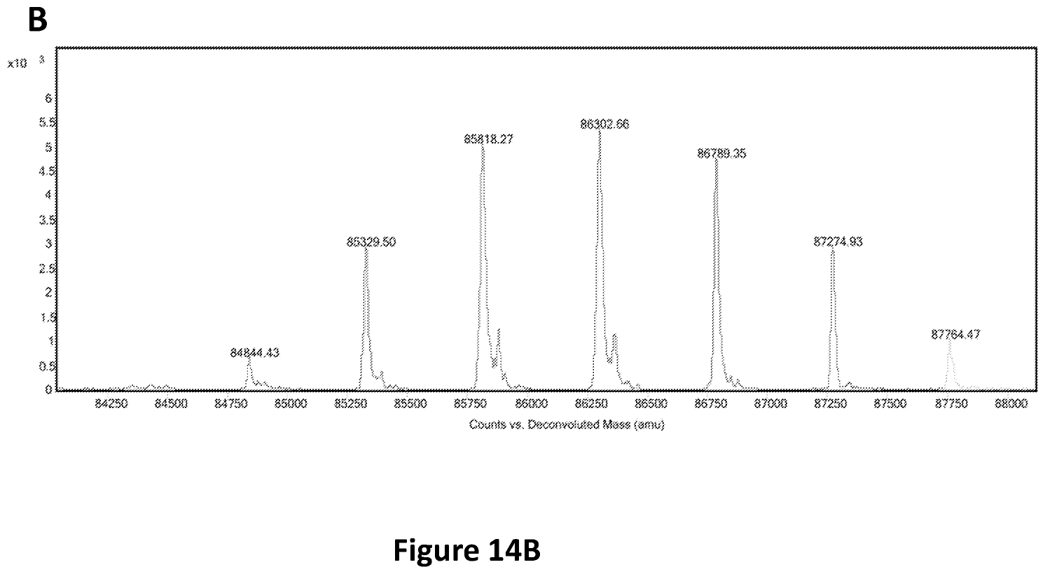

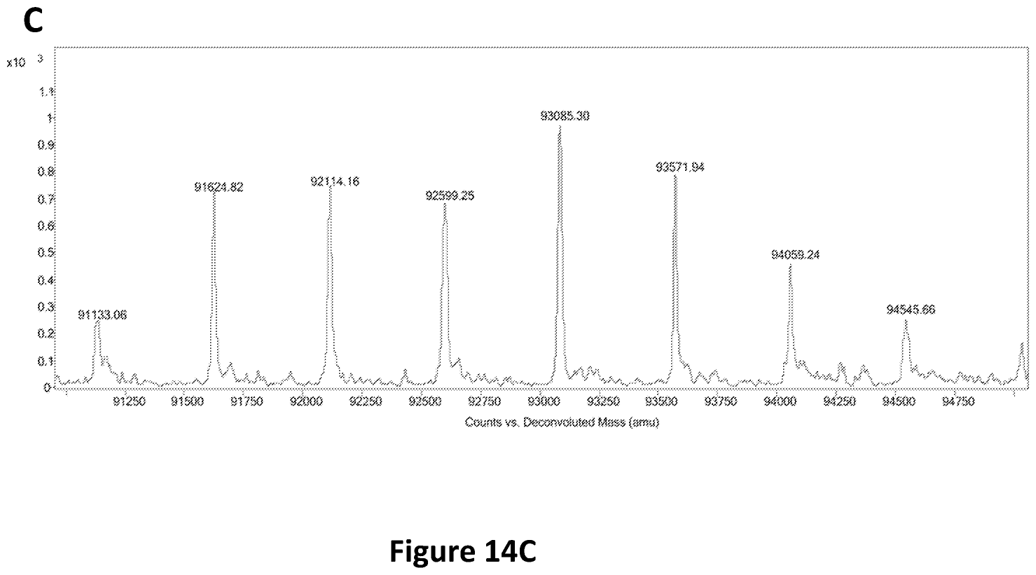

After confirming the correct immunological reactivity for each of the K. pneumoniae O-antigen modified EPA carrier protein, we selected four samples for further analysis via mass spectrometry. The O1v1-EPA, O2v1-EPA, O2v2-EPA or the O3b-EPA modified carrier proteins were separated on either a C4 or C8 column and infused into an Agilent 6520 Q-TOF mass spectrometer. This method allows the user to measure the intact mass of the O1v1-EPA, O2v1-EPA, O2v2-EPA or the O3b-EPA modified carrier proteins, which further enables the user to calculate the mass of posttranslational modifications like glycosylation mass compositions, the number of O-antigen repeat units per protein and the amount of glycosylation on a specific glycoprotein. Importantly this technique analysis allows us to track these properties at a molecular resolution allowing even small changes in glycosylation, such as alterations of a single monosaccharide, to be detected and quantified. FIG. 11A and FIG. 11B show the intact protein mass spectrometry analysis of the MS1 mass spectrum for the O1v1-EPA modified carrier protein. The non-glycosylated modified EPA protein has a theoretical mass of 79,526.15 Da, which was not readily dateable in the MS1 spectrum. The O1v1-EPA modified carrier protein was observed in multiple states of increasing mass corresponding to the Olvl repeat unit, which has a mass of 324 Da corresponding to two galactose residues linked by glyosidic bonds. FIG. 12A and FIG. 12B show the intact protein mass spectrometry analysis of the MS1 mass spectrum for the O2v1-EPA modified carrier protein. Again, the non-glycosylated modified EPA protein has a theoretical mass of 79,526.15 Da, which was not readily dateable in the MS1 spectrum. The O2v1-EPA modified carrier protein was observed in multiple states of increasing mass corresponding to the O2v1 repeat unit, which has a mass of 324 Da corresponding to two galactose residues linked by glyosidic bonds. FIG. 13A and FIG. 13B show the intact protein mass spectrometry analysis of the MS1 mass spectrum for the O2v2-EPA modified carrier protein. Again, the non-glycosylated modified EPA protein has a theoretical mass of 79,526.15 Da, which was not readily dateable in the MS1 spectrum. The O2v2-EPA modified carrier protein was observed in multiple states of increasing mass corresponding to the O2v2 repeat unit, which has a mass of 486 Da corresponding to three galactose residues linked by glyosidic bonds. FIG. 14A, FIG. 14B, and FIG. 14C show the intact protein mass spectrometry analysis of the MS1 mass spectrum for the O3b-EPA modified carrier protein. Again, the non-glycosylated modified EPA protein has a theoretical mass of 79,526.15 Da, which was not readily dateable in the MS1 spectrum. The O3b-EPA modified carrier protein was observed in multiple states of increasing mass corresponding to the O3b repeat unit, which has a mass of 486 Da corresponding to three mannose residues linked by glyosidic bonds.

### Example 2.

Modified MrkA carrier proteins can be glycosylated with K. pneumoniae O-antigen polysaccharides PglS_{ADP1}. In addition to surface polysaccharide polymers, like capsular polysaccharide and O-antigen polysaccharide, K. pneumoniae also produces surface proteins that form polymers. These protein polymers include the type 1 and type 3 fimbriae, which are assembled via the chaperone-usher pilus pathway (Hultgren, S. J., Normark, S. & Abraham, S. N., 1991). The major pilus subunit of the type 3 fimbriae from K. pneumoniae is the MrkA protein (Langstraat, J., Bohse, M. & Clegg, S., 2001), which has been shown to be highly conserved among K. pneumoniae isolates (Wang, Q. et al., 2016). MrkA in its monomeric form is not soluble; however, like type I pili proteins can be self-complemented to become soluble in its monomeric form by donor strand self-complementation (Walczak, M. J., Puorger, C., Glockshuber, R. & Wider, G., 2014). As such, we generated modified MrkA carrier proteins to self-complement by translationally fusing a hexaglycine linker and a duplicated MrkA N-terminal donor strand to the C-terminus of the MrkA protein. The flexible hexaglycine linker allows for the duplicated n-terminal donor strand to maintain stability of monomeric MrkA. In addition, we replaced the native signal peptide of MrkA with the DsbA signal peptide. The MrkA constructs were further modified to contain a glycine-glycine-glycine-serine linker and PglS_{ADP1}-dependent, O-linked glycosylation recognition motif that consists of either the ComP₁₁₀₂₆₄Δ28 fragment:

(SEQ ID NO: 8)
AYTDYTVRSRVTEGLTTASAMKATVSENIMNAGGTSMPSSGNCTGVTQIA
SGASAATTNVASAQCSDSDGVITVTMTDKAKGVSIKLTPSFSSTGSVGWK
CTTSSDKKYVPSECRGT

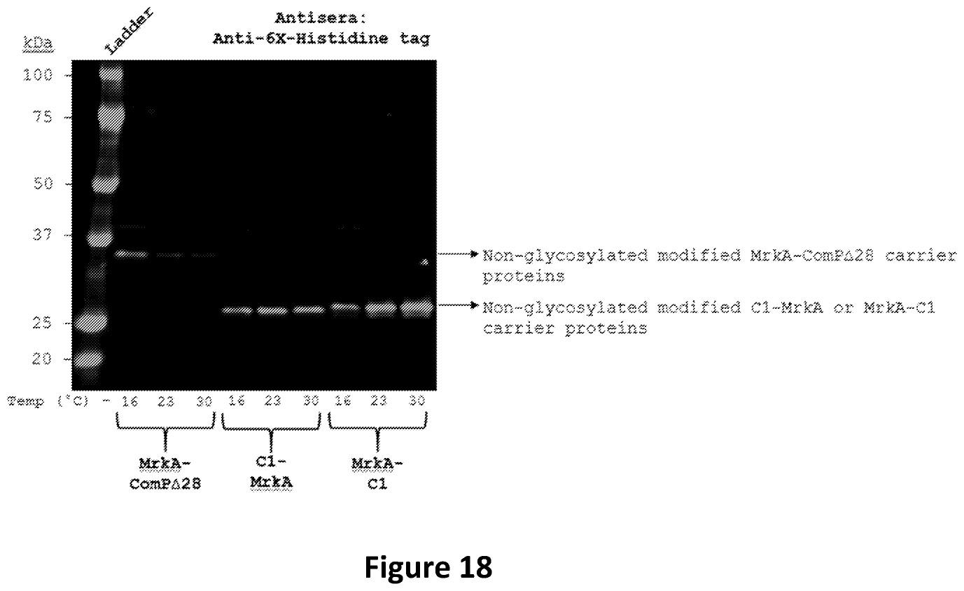

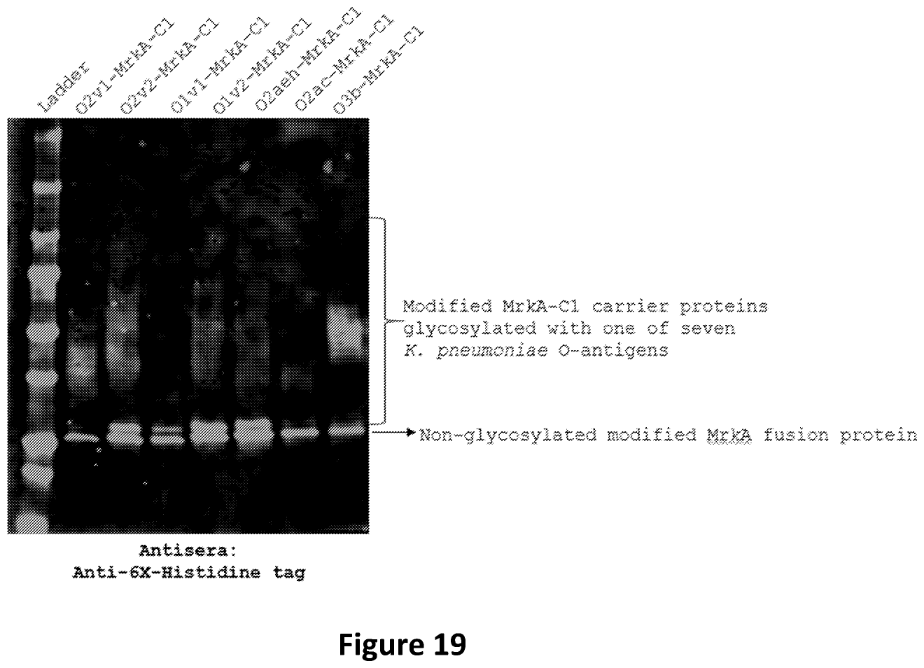

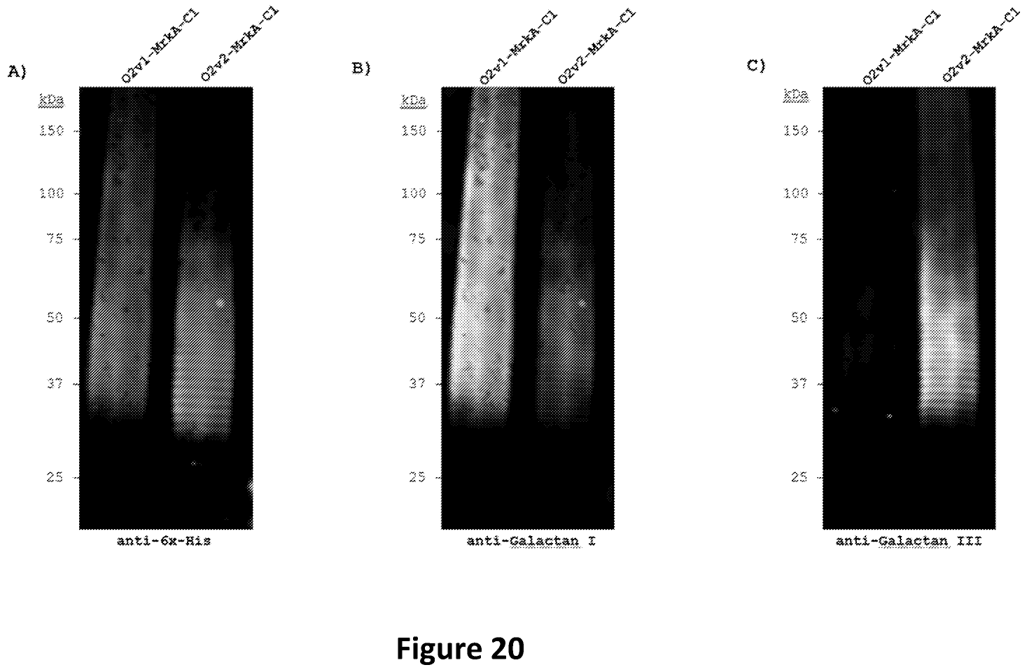

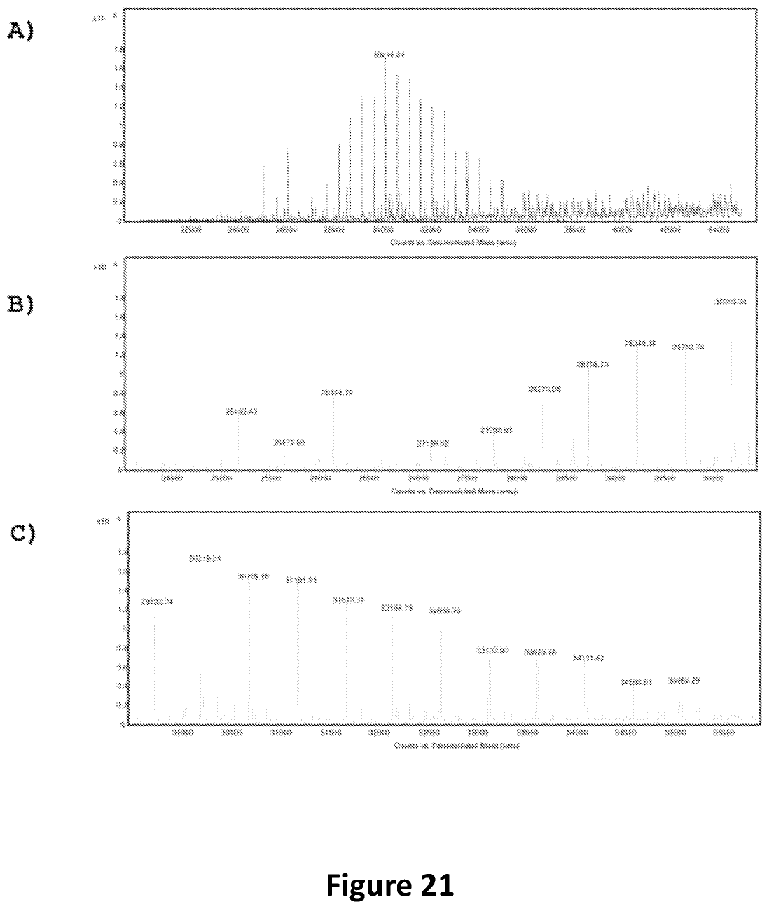

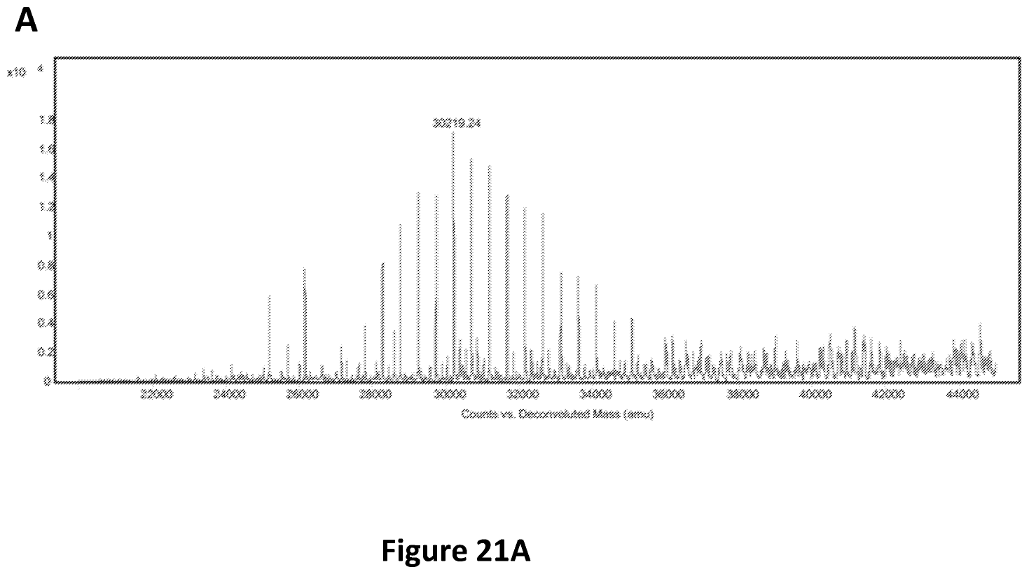

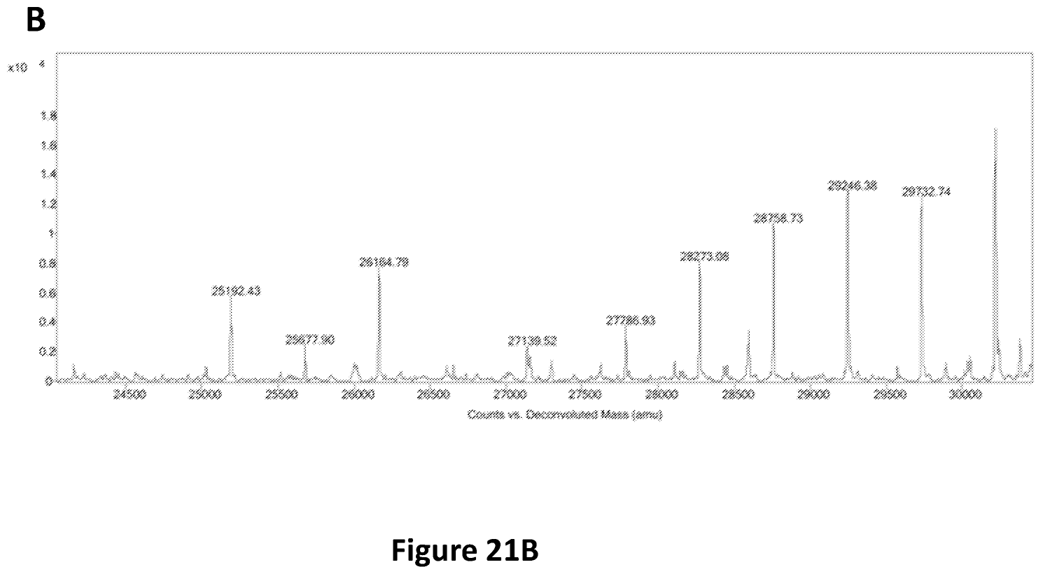

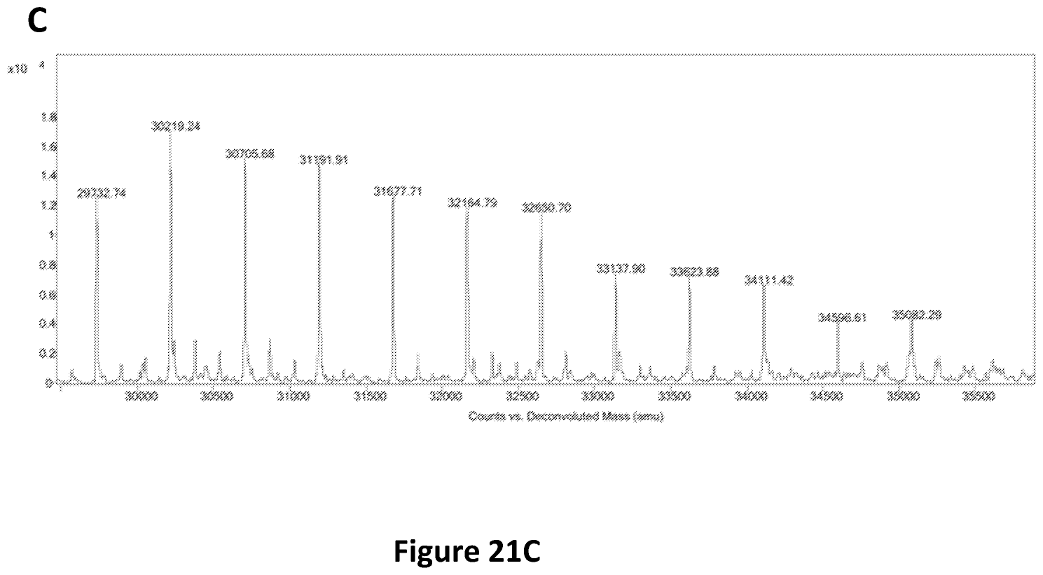

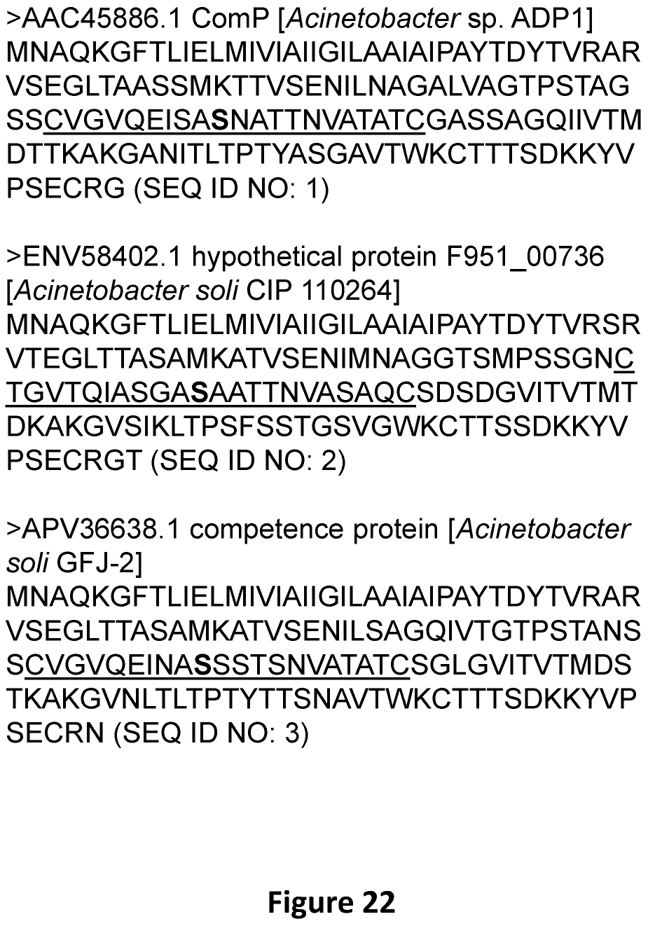

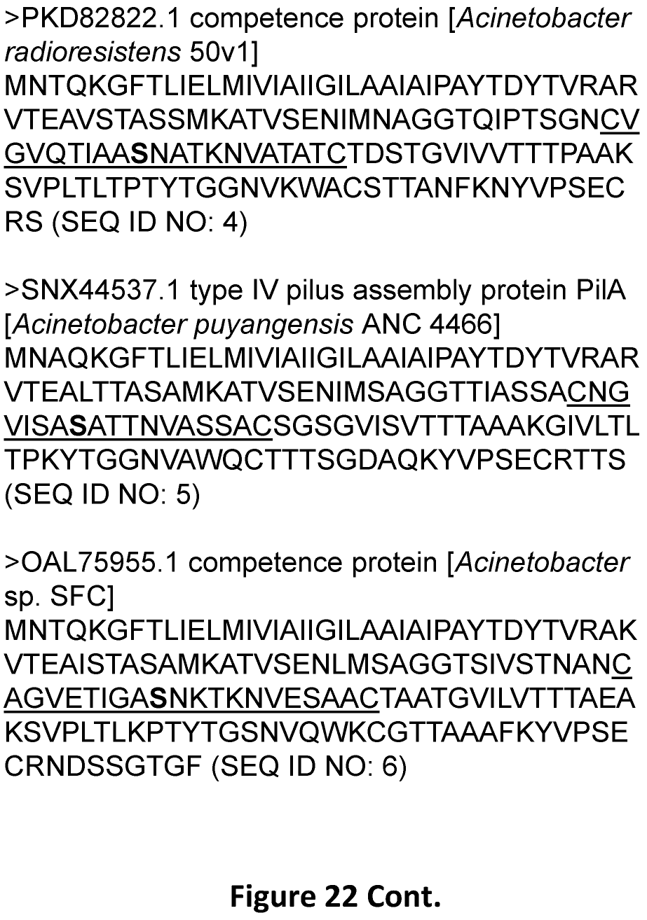

or the C1 fragment of ComP₁₁₀₂₆₄: GNCTGVTQIASGASAATTNVASAQC (SEQ ID NO: 32). Last all modified MrkA carrier proteins contain a hexahistidine tag. FIG. 15, FIG. 16, and FIG. 17 show schematics for the modified MrkA carrier proteins and the FASTA amino acid sequence for exemplary modified MrkA carrier proteins used in this study. First, we demonstrated the modified MrkA carrier protein could be expressed as a stable, soluble product in its monomeric form and be directed to the periplasm. To this end, we expressed the modified MrkA carrier protein in E. coli HSTO8 cells (Stellar Cells from Takara bio) at multiple temperatures and subsequently extracted periplasmic contents using an osmotic shock protocol. As seen in FIG. 18, the modified MrkA carrier protein containing the ComPΔ28 tag was poorly expressed or unstable as determined from western blot probing for the protein with anti-His antisera. Both the C1-MrkA and MrkA-C1 modified carrier proteins were detectable by western blot with the MrkA-C1 modified carrier protein appearing to be the most efficiently express construct at 30° C. After validating monomeric stability and periplasmic localization, we next combined the modified MrkA carrier protein with a plasmid expressing PglS_{ADP1} and a plasmid expressing a K. pneumoniae O-antigen in the CLM24 strain of E. coli. As seen in FIG. 19, the modified MrkA-C1 carrier protein appeared to be glycosylated with at least the O2v1, the O2v2, the O1v2, the O2aeh, the O2ac, and the O3 b O-antigens as determined by western blotting probing for anti-His immunoreactivity as indicated by the higher molecular weight laddering detected above the non-glycosylated form. Next, we purified the O2v1-MrkA and the O2v2-MrkA modified carrier proteins and further analyzed these glycoproteins via SDS-PAGE and western blotting. As seen in FIG. 20A, the O2v1-MrkA and the O2v2 -MrkA modified carrier proteins exhibited a laddering like electrophoretic mobility when probed with the anti-his antisera via western blot. Furthermore, the same samples were examined via western blot probing with anti-sera specific for D-galactan I or D-galactan III epitopes. As seen in FIG. 20b, the O2v1-MrkA modified carrier protein was more immunoreactive with the anti-D-galactan I antisera in comparison to O2v2-MrkA. This is expected as there are likely some unmodified O2v1 (O2a) repeating units within the O2v2 (O2afg) antigen. However, when the O2v1-MrkA and the O2v2 -MrkA modified carrier protein samples were probed with the anti-D-galactan-III antisera as shown in FIG. 20C, only the O2v2-MrkA modified carrier protein displayed immunoreactivity confirming that the D-galactan-III epitope is not present in the O2v1 antigen (O2a).

The breadth and scope of the present disclosure should not be limited by any of the above-described exemplary embodiments, but should be defined only in accordance with the following claims and their equivalents.

The present invention can be defined in any of the following numbered embodiment paragraphs:

1. A bioconjugate comprising a K. pneumoniae O-antigen covalently linked to a fusion protein, wherein the fusion protein comprises a ComP protein or a glycosylation tag fragment thereof,

optionally, wherein the O-antigen has not been derivatized by:
i) being subject to oxidation/reduction procedures;
ii) activated with 1-Cyano-4-Dimethylaminopyridine Tetrafluoroborate (CDAP);
iii) the addition of primary amines; and/or
iv) the addition of diamine spacer molecules, further optionally, wherein the O-antigen is underivatized;

optionally, wherein the O-antigen is a native O-antigen; and/or

optionally, wherein the bioconjugate is immunogenic.

2. The bioconjugate of Embodiment 1, wherein:

the K. pneumoniae O-antigen is selected from the group consisting of O1, O2, O3, O4, O5, O7, O8 and O12;

the K. pneumoniae O-antigen is selected from the group consisting of O1v1, O1v2, O2v1, O2v2, O3, O3a, and O3b; and/or

the K. pneumoniae O-antigen is selected from the group consisting of O1afg, O2afg, O2aeh, and O2ac.

3. The bioconjugate of Embodiment 1 or 2, wherein the fusion protein comprises a ComP protein or a glycosylation tag fragment thereof attached to a heterologous carrier protein;

optionally, wherein the ComP protein or a glycosylation tag fragment thereof is attached to the heterologous carrier protein via an amino acid linker;

optionally, wherein the ComP protein or a glycosylation tag fragment thereof is located in the fusion protein C-terminal to the heterologous carrier protein;

optionally, wherein the ComP protein or a glycosylation tag fragment thereof is located in the fusion protein N-terminal to the heterologous carrier protein; and/or

optionally, wherein the fusion protein comprises a signal peptide.

4. The bioconjugate of Embodiment 3, wherein the fusion protein comprises a Pseudomonas aeruginosa exotoxin A (EPA) carrier protein, a CRM₁₉₇ carrier protein, a tetanus toxin C fragment carrier protein, or a K. pneumoniae MrkA carrier protein;

optionally, wherein the MrkA carrier protein comprises a modified MrkA variant that is self-complemented by translationally fusing a hexaglycine linker and a duplicated MrkA N-terminal donor strand to the C-terminus of the MrkA protein;

optionally, wherein the MrkA carrier protein comprises a native MrkA signal peptide or comprises a DsbA protein signal peptide in place of the MrkA native signal peptide; and/or

optionally, wherein the MrkA carrier protein comprises a glycine-glycine-glycine-serine linker linking it to ComP protein or a glycosylation tag fragment thereof.

5. The bioconjugate of any one of Embodiments 1 to 4, wherein the glycosylation tag fragment of ComP comprises or consists of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2]; SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO: 58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7]; SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11];

optionally, wherein the ComP glycosylation tag does not comprise a methionine residue in a position corresponding to the conserved methionine residue at position 104 of SEQ ID NO: [2] (ComP110264: ENV58402.1); and/or

optionally, wherein the amino acid sequence of the ComP glycosylation tag does not extend in the C-terminus direction beyond the amino acid residue corresponding to position 103 of SEQ ID NO: [2] (ComP110264: ENV58402.1).

6. The bioconjugate of Embodiment 5, wherein the glycosylation tag fragment of ComP comprises or consists of SEQ ID NO: 32 [C1].

7. The bioconjugate of any one of Embodiments 1 to 6, wherein the fusion protein comprises:

SEQ ID NO: 122 (DsbASP-ComP₁₁₀₂₆₄C1_fragment-GGGS-MrkA-GGGGGG-donor_strand);

SEQ ID NO: 124 (DsbASP-MrkA-GGGGGG-donor_strand-GGGS-ComP₁₀₂₆₄C1_fragment);

SEQ ID NO: 121 (DsbASP-ComP₁₁₀₂₆₄C1_fragment-GGGS-MrkA-GGGGGG-donor_strand-His); or

SEQ ID NO: 123 (DsbASP-MrkA-GGGGGG-donor_strand-GGGS-ComP₁₁₀₂₆₄C1_fragment-His).

8. The bioconjugate of any one of Embodiments 1 to 7, wherein the bioconjugate is a conjugate vaccine.

9. The bioconjugate of any one of Embodiments 1 to 7, for use as a conjugate vaccine.

10. The bioconjugate of any one of Embodiments 1 to 9, wherein the bioconjugate is produced in vivo; optionally in a bacterial cell.

11. A conjugate vaccine composition comprising the bioconjugate of any one of Embodiments 1 to 10.

12. The conjugate vaccine composition of Embodiment 11, wherein the conjugate vaccine composition is a multivalent vaccine comprising at least two, three, four, five, six, or seven of the bioconjugates, each comprising a different K. pneumoniae O-antigen.

13. The conjugate vaccine composition of Embodiment 12, wherein the conjugate vaccine is a multivalent vaccine comprising seven of the bioconjugates each comprising a different K. pneumoniae O-antigen.

14. The conjugate vaccine composition of Embodiment 13, comprising:
(i) a bioconjugate comprising an O1v1 antigen;
(ii) a bioconjugate comprising an O1v2 antigen;
(iii) a bioconjugate comprising an O2v1 antigen;
(iv) a bioconjugate comprising an O2v2 antigen;
(v) a bioconjugate comprising an O3 antigen;
(vi) a bioconjugate comprising an O3b antigen; and
(vii) a bioconjugate comprising an O5 antigen.

15. The conjugate vaccine composition of any one of Embodiments 11 to 14, further comprising an adjuvant.

16. A fusion protein comprising ComP or a glycosylation tag fragment thereof and a Pseudomonas aeruginosa exotoxin A (EPA) carrier protein, a CRM₁₉₇ carrier protein, a tetanus toxin C fragment carrier protein, or a K. pneumoniae MrkA carrier protein;

optionally, wherein the MrkA carrier protein comprises a modified MrkA variant that is self-complemented by translationally fusing a hexaglycine linker and a duplicated MrkA N-terminal donor strand to the C-terminus of the MrkA protein;

optionally, wherein the MrkA carrier protein comprises a native MrkA signal peptide or comprises a DsbA protein signal peptide in place of the MrkA native signal peptide; and/or

optionally, wherein the MrkA carrier protein comprises a glycine-glycine-glycine-serine linker linking it to ComP protein or a glycosylation tag fragment thereof.

17. The fusion protein of Embodiment 16, wherein the fusion protein is covalently linked to a K. pneumoniae O-antigen;

optionally, wherein the O-antigen has not been derivatized by:
i) being subject to oxidation/reduction procedures;
ii) activated with 1-Cyano-4-Dimethylaminopyridine Tetrafluoroborate (CDAP);
iii) the addition of primary amines; and/or
iv) the addition of diamine spacer molecules, further optionally, wherein the O-antigen is underivatized;

optionally, wherein the O-antigen is a native O-antigen; and/or

optionally, wherein the bioconjugate is immunogenic.

18. The fusion protein of Embodiment 17, wherein:

the K. pneumoniae O-antigen is selected from the group consisting of O1, O2, O3, O4, O5, O7, O8 and O12;

the K. pneumoniae O-antigen is selected from the group consisting of O1v1, O1v2, O2v1, O2v2, O3, O3a, and O3b; and/or

the K. pneumoniae O-antigen is selected from the group consisting of O1afg, O2afg, O2aeh, and O2ac.

19. A method of producing a bioconjugate, the method comprising covalently linking a K. pneumoniae O-antigen to a fusion protein with a PglS oligosaccharyltransferase (OTase),

wherein the fusion protein comprises a ComP protein or a glycosylation tag fragment thereof;

optionally, wherein the ComP protein or glycosylation tag fragment thereof is linked to a heterologous carrier protein.

20. A method of inducing a host immune response against K. pneumoniae, the method comprising administering to a subject in need of the immune response an effective amount of the conjugate vaccine composition of any one of Embodiments 11 to 15;

optionally, wherein the subject is a human.

21. The method of Embodiment 20, wherein the immune response is an antibody response.

22. The method of Embodiment 20, wherein the immune response is selected from the group consisting of an innate response, an adaptive response, a humoral response, an antibody response, cell mediated response, a B cell response, a T cell response, cytokine upregulation or downregulation, immune system cross-talk, and a combination of two or more of said immune responses.

23. The method of Embodiment 22, wherein the immune response is selected from the group consisting of an innate response, a humoral response, an antibody response, a T cell response, and a combination of two or more of said immune responses.

24. A method of preventing or treating a K. pneumoniae infection in a subject comprising administering to a subject in need thereof the bioconjugate of any one of Embodiments 1 to 10; optionally, wherein the subject is a human.

25. Use of the bioconjugate of any one of Embodiment s 1 to 10, the conjugate vaccine of any one of Embodiments 11 to 15, or the fusion protein of any one of Embodiments 16 to 18 to induce a host immune response against K. pneumoniae, prevent a K. pneumoniae infection, and/or treat a K. pneumoniae infection.

26. A method of producing a conjugate vaccine against K. pneumoniae infection, the method comprising:

(a) isolating the bioconjugate of any one of Embodiments 1 to 10; and

(b) combining the isolated bioconjugate with an adjuvant.

DSBA_{SP}-EPAΔE553-GGGS-COMP₁₁₀₂₆₄Δ28-HIS
(SEQ ID NO: 117)
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLETFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDISFSTRGTQNWTVERL
LQAHRQLEERGYVFVGYHGTFLEAAQSIVFGGVRARSQDLDAIWRGFYI
AGDPALAYGYAQDQEPDARGRIRNGALLRVYVPRSSLPGFYRTGLTLAA
PEAAGEVERLIGHPLPLRLDAITGPEEEGGRLTILGWPLAERTVVIPSA
IPTDPRNVGGDLDPSSIPDKEQAISALPDYASQPGKPPREDLKGGGSAY
TDYTVRSRVTEGLTTASAMKATVSENIMNAGGTSMPSSGNCTGVTQIAS
GASAATTNVASAQCSDSDGVITVTMTDKAKGVSIKLTPSFSSTGSVGWK
CTTSSDKKYVPSECRGTHHHHHH
DSBA_{SP}-EPAΔE553-GGGS-COMP₁₁₀₂₆₄28
(SEQ ID NO: 118)
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLETFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDISFSTRGTQNWTVERL
LQAHRQLEERGYVFVGYHGTFLEAAQSIVFGGVRARSQDLDAIWRGFYI
AGDPALAYGYAQDQEPDARGRIRNGALLRVYVPRSSLPGFYRTGLTLAA
PEAAGEVERLIGHPLPLRLDAITGPEEEGGRLTILGWPLAERTVVIPSA
IPTDPRNVGGDLDPSSIPDKEQAISALPDYASQPGKPPREDLKGGGSAY
TDYTVRSRVTEGLTTASAMKATVSENIMNAGGTSMPSSGNCTGVTQIAS
GASAATTNVASAQCSDSDGVITVTMTDKAKGVSIKLTPSFSSTGSVGWK
CTTSSDKKYVPSECRGT
DSBA_{SP}-MRKA-GGGGGG-DONOR_STRAND-GGGS-COMP₁₁₀₂₆₄Δ28-
HIS
(SEQ ID NO: 119)
MKKIWLALAGLVLAFSASAADTNVGGGQVNFFGKVTDVSCTVSVNGQGS
DANVYLSPVTLTEVKAAAADTYLKPKSFTIDVSNCQAADGTKQDDVSKL
GVNWTGGNLLAGATSKQQGYLANTEASGAQNIQLVLSTDNATALTNKII
PGDSTQPKAKGDASAVADGARFTYYVGYATSAPTTVTTGVVNSYATYEI
TYQGGGGGGADTNVGGGQVNFFGKVTDVSGGGSAYTDYTVRSRVTEGLT
TASAMKATVSENIMNAGGTSMPSSGNCTGVTQIASGASAATTNVASAQC
SDSDGVITVTMTDKAKGVSIKLTPSFSSTGSVGWKCTTSSDKKYVPSEC
RGTHHHHHH
DSBA_{SP}-MRKA-GGGGGG-DONOR_STRAND-GGGS-COMP₁₁₀₂₆₄Δ28
(SEQ ID NO: 120)
MKKIWLALAGLVLAFSASAADTNVGGGQVNFFGKVTDVSCTVSVNGQGS
DANVYLSPVTLTEVKAAAADTYLKPKSFTIDVSNCQAADGTKQDDVSKL
GVNWTGGNLLAGATSKQQGYLANTEASGAQNIQLVLSTDNATALTNKII
PGDSTQPKAKGDASAVADGARFTYYVGYATSAPTTVTTGVVNSYATYEI
TYQGGGGGGADTNVGGGQVNFFGKVTDVSGGGSAYTDYTVRSRVTEGLT
TASAMKATVSENIMNAGGTSMPSSGNCTGVTQIASGASAATTNVASAQC
SDSDGVITVTMTDKAKGVSIKLTPSFSSTGSVGWKCTTSSDKKYVPSEC
RGT
DSBA_{SP}-COMP₁₁₀₂₆₄C1_FRAGMENT-GGGS-MRKA-GGGGGG-
DONOR_STRAND-HIS
(SEQ ID NO: 121)
MKKIWLALAGLVLAFSASAGNCTGVTQIASGASAATTNVASAQCGGGSA
DTNVGGGQVNFFGKVTDVSCTVSVNGQGSDANVYLSPVTLTEVKAAAAD
TYLKPKSFTIDVSNCQAADGTKQDDVSKLGVNWTGGNLLAGATSKQQGY
LANTEASGAQNIQLVLSTDNATALTNKIIPGDSTQPKAKGDASAVADGA
RFTYYVGYATSAPTTVTTGVVNSYATYEITYQGGGGGGADTNVGGGQVN
FFGKVTDVSGSGHHHHHH
DSBA_{SP}-COMP₁₁₀₂₆₄C1_FRAGMENT-GGGS-MRKA-GGGGGG-
DONOR_STRAND
(SEQ ID NO: 122)
MKKIWLALAGLVLAFSASAGNCTGVTQIASGASAATTNVASAQCGGGSA
DTNVGGGQVNFFGKVTDVSCTVSVNGQGSDANVYLSPVTLTEVKAAAAD
TYLKPKSFTIDVSNCQAADGTKQDDVSKLGVNWTGGNLLAGATSKQQGY
LANTEASGAQNIQLVLSTDNATALTNKIIPGDSTQPKAKGDASAVADGA
RFTYYVGYATSAPTTVTTGVVNSYATYEITYQGGGGGGADTNVGGGQVN
FFGKVTDVSGSG
DSBA_{SP}-MRKA-GGGGGG-DONOR_STRAND-GGGS-
COMP₁₁₀₂₆₄C1_FRAGMENT-HIS
(SEQ ID NO: 123)
MKKIWLALAGLVLAFSASAADTNVGGGQVNFFGKVTDVSCTVSVNGQGS
DANVYLSPVTLTEVKAAAADTYLKPKSFTIDVSNCQAADGTKQDDVSKL
GVNWTGGNLLAGATSKQQGYLANTEASGAQNIQLVLSTDNATALTNKII
PGDSTQPKAKGDASAVADGARFTYYVGYATSAPTTVTTGVVNSYATYEI
TYQGGGGGGADTNVGGGQVNFFGKVTDVSGGGSGNCTGVTQIASGASAA
TTNVASAQCHHHHHH
DSBA_{SP}-MRKA-GGGGGG-DONOR_STRAND-GGGS-
COMP₁₁₀₂₆₄C1_FRAGMENT
(SEQ ID NO: 124)
MKKIWLALAGLVLAFSASAADTNVGGGQVNFFGKVTDVSCTVSVNGQGS
DANVYLSPVTLTEVKAAAADTYLKPKSFTIDVSNCQAADGTKQDDVSKL
GVNWTGGNLLAGATSKQQGYLANTEASGAQNIQLVLSTDNATALTNKII
PGDSTQPKAKGDASAVADGARFTYYVGYATSAPTTVTTGVVNSYATYEI
TYQGGGGGGADTNVGGGQVNFFGKVTDVSGGGSGNCTGVTQIASGASAA
TTNVASAQC

TABLE 1

| ID | SEQ ID NO: | ComP₁₁₀₂₆₄ fragments |
|---|---|---|
| A1 | 30 | ₆₇SSGNCTGVTQIASGASAATTNVASA₉₁ |
| B1 | 31 | ₆₈SGNCTGVTQIASGASAATINVASAQ₉₂ |
| C1 | 32 | ₆₉GNCTGVTQIASGASAATTNVASAQC₉₃ |
| D1 | 33 | ₇₀NCTGVTQIASGASAATTNVASAQCS₉₄ |
| E1 | 34 | ₇₁CTGVTQIASGASAATTNVASAQCSD₉₅ |
| F1 | 35 | ₇₂TGVTQIASGASAATINVASAQCSDS₉₆ |
| G1 | 36 | ₇₃GVTQIASGASAATTNVASAQCSDSD₉₇ |
| H1 | 37 | ₇₄VTQIASGASAATTNVASAQCSDSDG₉₈ |
| A2 | 38 | ₇₅TQIASGASAATTNVASAQCSDSDGV₉₉ |
| C2 | 39 | ₆₂GTSMPSSGNCTGVTQIASGASAATTNVASA₉₁ |
| D2 | 40 | ₆₃TSMPSSGNCTGVTQIASGASAATTNVASAQ₉₂ |
| E2 | 41 | ₆₄SMPSSGNCTGVTQIASGASAATINVASAQC₉₃ |
| F2 | 42 | ₆₅MPSSGNCTGVTQIASGASAATTNVASAQCS₉₄ |
| G2 | 43 | ₆₆PSSGNCTGVTQIASGASAATTNVASAQCSD₉₅ |
| H2 | 44 | ₆₇SSGNCTGVTQIASGASAATINVASAQCSDS₉₆ |
| A3 | 45 | ₆₈SGNCTGVTQIASGASAATINVASAQCSDSD₉₇ |
| B3 | 46 | ₆₉GNCTGVTQIASGASAATINVASAQCSDSDG₉₈ |
| C3 | 47 | ₇₀NCTGVTQIASGASAATTNVASAQCSDSDGV₉₉ |
| E3 | 48 | ₇₂TGVTQIASGASAATTNVASAQCSDSDGVIT₁₀₁ |
| F3 | 49 | ₇₃GVTQIASGASAATTNVASAQCSDSDGVITV₁₀₂ |
| G3 | 50 | ₇₄VTQIASGASAATTNVASAQCSDSDGVITVT₁₀₃ |
| H3 | 51 | ₇₅TQIASGASAATTNVASAQCSDSDGVITVTM₁₀₄ |
| A4 | 52 | ₇₆QIASGASAATTNVASAQCSDSDGVITVTMT₁₀₅ |
| B4 | 53 | ₅₇IMNAGGTSMPSSGNCTGVTQIASGASAATTNVASA₉₁ |
| C4 | 54 | ₅₈MNAGGTSMPSSGNCTGVTQIASGASAATTNVASAQ₉₂ |
| D4 | 55 | ₅₉NAGGISMPSSGNCTGVTQIASGASAATTNVASAQC₉₃ |
| E4 | 56 | ₆₀AGGISMPSSGNCTGVTQIASGASAATTINVASAQCS₉₄ |
| F4 | 57 | ₆₁GGTSMPSSGNCTGVTQIASGASAATTNVASAQCSD₉₅ |
| G4 | 58 | ₆₂GTSMPSSGNCTGVTQIASGASAATINVASAQCSDS₉₆ |
| A5 | 59 | ₆₄SMPSSGNCTGVTQIASGASAATTNVASAQCSDSDG₉₈ |
| B5 | 60 | ₆₅MPSSGNCTGVTQIASGASAATTNVASAQCSDSDGV₉₉ |
| D5 | 61 | ₆₇SSGNCTGVTQIASGASAATTNVASAQCSDSDGVIT₁₀₁ |
| E5 | 62 | ₆₈SGNCTGVTQIASGASAATTNVASAQCSDSDGVITV₁₀₂ |
| F5 | 63 | ₆₉GNCTGVTQIASGASAATTNVASAQCSDSDGVITVT₁₀₃ |
| G5 | 64 | ₇₀NCTGVTQIASGASAATTNVASAQCSDSDGVITVTM₁₀₄ |
| H5 | 65 | ₇₁CTGVTQIASGASAATTNVASAQCSDSDGVITVTMT₁₀₅ |
| A6 | 66 | ₇₂TGVTQIASGASAATTNVASAQCSDSDGVITVTMTD₁₀₆ |
| B6 | 67 | ₇₃GVTQIASGASAATTNVASAQCSDSDGVITVTMTDK₁₀₇ |
| C6 | 68 | ₇₄VTQIASGASAATTNVASAQCSDSDGVITVTMTDKA₁₀₈ |
| D6 | 69 | ₇₅TQIASGASAATTNVASAQCSDSDGVITVTMTDKAK₁₀₉ |
| F6 | 70 | ₅₂TVSENIMNAGGTSMPSSGNCTGVTQIASGASAATINVASA₉₁ |
| G6 | 71 | ₅₃VSENIMNAGGTSMPSSGNCTGVTQIASGASAATTNVASAQ₉₂ |
| H6 | 72 | ₅₄SENIMNAGGTSMPSSGNCTGVTOIASGASAATTNVASAQC₉₃ |
| B7 | 73 | ₅₆NIMNAGGTSMPSSGNCTGVTQIASGASAATTNVASAQCSD₉₅ |
| C7 | 74 | ₅₇IMNAGGTSMPSSGNCTGVTQIASGASAATTNVASAQCSDS₉₆ |
| D7 | 75 | ₅₈MNAGGTSMPSSGNCTGVTQIASGASAATTNVASAQCSDSD₉₇ |
| E7 | 76 | ₅₉NAGGTSMPSSGNCTGVTQIASGASAATINVASAQCSDSDG₉₈ |
| E7 | 77 | ₆₀AGGTSMPSSGNCTGVTOIASGASAATTNVASAQCSDSDGV₉₉ |
| A8 | 78 | ₆₃TSMPSSGNCTGVTQIASGASAATTNVASAQCSDSDGVITV₁₀₂ |
| B8 | 79 | ₆₄SMPSSGNCTGVTQIASGASAATTNVASAQCSDSDGVITVT₁₀₃ |
| C8 | 80 | ₆₅MPSSGNCTGVTQIASGASAATTNVASAQCSDSDGVITVTM₁₀₄ |
| D8 | 81 | ₆₆PSSGNCTGVTQIASGASAATTNVASAQCSDSDGVITVTMT₁₀₅ |
| E8 | 82 | ₆₇SSGNCTGVTQIASGASAATTNVASAQCSDSDGVITVTMTD₁₀₆ |
| F8 | 83 | ₆₈SGNCTGVTQIASGASAATTNVASAQCSDSDGVITVTMTDK₁₀₇ |
| G8 | 84 | ₆₉GNCTGVTQIASGASAATTNVASAQCSDSDGVITVTMTDKA₁₀₈ |
| H8 | 85 | ₇₀NCTGVTQIASGASAATTNVASAQCSDSDGVITVTMTDKAK₁₀₉ |
| A9 | 86 | ₇₁CTGVTQIASGASAATTNVASAQCSDSDGVITVTMTDKAKG₁₁₀ |
| B9 | 87 | ₇₂TGVTQIASGASAATTNVASAQCSDSDGVITVTMTDKAKGV₁₁₁ |
| C9 | 88 | ₇₃GVTQIASGASAATTNVASAQCSDSDGVITVTMTDKAKGVS₁₁₂ |
| D9 | 89 | ₇₄VTQIASGASAATTNVASAQCSDSDGVITVTMTDKAKGVSI₁₁₃ |
| E9 | 90 | ₇₅TQIASGASAATTNVASAQCSDSDGVITVTMTDKAKGVSIK₁₁₄ |
| H9 | 91 | ₄₈AMKATVSENIMNAGGTSMPSSGNCTGVTQIASGASAATTNVASAQ₉₂ |
| A10 | 92 | ₄₉MKATVSENIMNAGGTSMPSSGNCTGVTQIASGASAATTNVASAQC₉₃ |
| B10 | 93 | ₅₀KATVSENIMNAGGTSMPSSGNCTGVTQIASGASAATTNVASAQCS₉₄ |
| C10 | 94 | ₅₁ATVSENIMNAGGTSMPSSGNCTGVTQIASGASAATTNVASAQCSD₉₅ |
| D10 | 95 | ₅₂TVSENIMNAGGTSMPSSGNCTGVTQIASGASAATINVASAQCSDS₉₆ |
| F10 | 96 | ₅₄SENIMNAGGTSMPSSGNCTGVTQIASGASAATTNVASAQCSDSDG₉₈ |
| G10 | 97 | ₅₅ENIMNAGGTSMPSSGNCTGVTQIASGASAATTNVASAQCSDSDGV₉₉ |
| H10 | 98 | ₅₆NIMNAGGTSMPSSGNCTGVTQIASGASAATTNVASAQCSDSDGVI₁₀₀ |
| A11 | 99 | ₅₇IMNAGGTSMPSSGNCTGVTQIASGASAATTNVASAQCSDSDGVIT₁₀₁ |
| B11 | 100 | ₅₈MNAGGTSMPSSGNCTGVTQIASGASAATTNVASAQCSDSDGVITV₁₀₂ |
| C11 | 101 | ₅₉NAGGTSMPSSGNCTGVTQIASGASAATTNVASAQCSDSDGVITVT₁₀₃ |
| D11 | 102 | ₆₀AGGTSMPSSGNCTGVTQIASGASAATTNVASAQCSDSDGVITVTM₁₀₄ |
| E11 | 103 | ₆₁GGTSMPSSGNCTGVTQIASGASAATTNVASAQCSDSDGVITVTMT₁₀₅ |
| F11 | 104 | ₆₂GTSMPSSGNCTGVTQIASGASAATTNVASAQCSDSDGVITVTMTD₁₀₆ |
| H11 | 105 | ₆₄SMPSSGNCTGVTQIASGASAATTNVASAQCSDSDGVITVTMTDKA₁₀₈ |
| A12 | 106 | ₆₅MPSSGNCTGVTQIASGASAATTNVASAQCSDSDGVITVTMTDKAK₁₀₉ |
| B12 | 107 | ₆₆PSSGNCTGVTQIASGASAATTNVASAQCSDSDGVITVTMTDKAKG₁₁₀ |
| C12 | 108 | ₆₇SSGNCTGVTQIASGASAATTNVASAQCSDSDGVITVTMTDKAKGV₁₁₁ |
| D12 | 109 | ₆₈SGNCTGVTQIASGASAATTNVASAQCSDSDGVITVTMTDKAKGVS₁₁₂ |
| E12 | 110 | ₆₉GNCTGVTQIASGASAATINVASAQCSDSDGVITVTMTDKAKGVSI₁₁₃ |
| F12 | 111 | ₇₀NCTGVTQIASGASAATTNVASAQCSDSDGVITVTMTDKAKGVSIK₁₁₄ |
| G12 | 112 | ₇₁CTGVTQIASGASAATTNVASAQCSDSDGVITVTMTDKAKGVSIKL₁₁₅ |
| H12 | 113 | ₇₂TGVTQIASGASAATTNVASAQCSDSDGVITVTMTDKAKGVSIKLT₁₁₆ |

### REFERENCES

1. Resistance, I. C. G. o. A. NO TIME TO WAIT: SECURING THE FUTURE FROM DRUG-RESISTANT INFECTIONS, <https://www.who.int/docs/default-source/documents/no -time-to-wait-securing-the-future-from-drug-resistant-infections-en.pdf?sfvrsn=5b424d7_6>(2019).

2. CDC. ANTIBIOTIC RESISTANCE THREATS IN THE UNITED STATES 2019, <https://www.cdc.gov/drugresistance/pdf/threats-report/2019-ar-threats-report-508.pdf>(2019).

3. CDC. ANTIBIOTIC RESISTANCE THREATS in the United States, 2013, <https://www.cdc.gov/drugresistance/pdf/ar-threats-2013-508.pdf>(2013).

4. Magill, S. S. et al. Changes in Prevalence of Health Care-Associated Infections in U.S. Hospitals. N Engl J Med 379, 1732-1744, doi:10.1056/NEJMoa1801550 (2018).

5. Cassini, A. et al. Attributable deaths and disability-adjusted life-years caused by infections with antibiotic-resistant bacteria in the EU and the European Economic Area in 2015: a population-level modelling analysis. Lancet Infect Dis 19, 56-66, doi:10.1016/51473-3099(18)30605-4 (2019).

6. Policy, N. I. o. H. 0. o. S. CHILDHOOD Hib VACCINES: NEARLY ELIMINATING THE THREAT OF BACTERIAL MENINGITIS, <https://www.nih.gov/sites/default/files/about -nih/impact/childhood-hib-vaccines-case-study.pdf>.

7. Daniels, C. C., Rogers, P. D. & Shelton, C. M. A Review of Pneumococcal Vaccines: Current Polysaccharide Vaccine Recommendations and Future Protein Antigens. J Pediatr Pharmacol Ther 21, 27-35, doi:10.5863/1551-6776-21.1.27 (2016).

8. Hampton, L. M. et al. Prevention of antibiotic-nonsusceptible Streptococcus pneumoniae with conjugate vaccines. J Infect Dis 205, 401-411, doi:10.1093/infdis/jir755 (2012).

9. Tomczyk, S. et al. Prevention of Antibiotic-Nonsusceptible Invasive Pneumococcal Disease With the 13-Valent Pneumococcal Conjugate Vaccine. Clin Infect Dis 62, 1119-1125, doi:10.1093/cid/ciw067 (2016).

10. Cohen, R., Cohen, J. F., Chalumeau, M. & Levy, C. Impact of pneumococcal conjugate vaccines for children in high- and non-high-income countries. Expert Rev Vaccines 16, 625-640, doi:10.1080/14760584.2017.1320221 (2017).

11. Avci, F. Y., Li, X., Tsuji, M. & Kasper, D. L. A mechanism for glycoconjugate vaccine activation of the adaptive immune system and its implications for vaccine design. Nat Med 17, 1602-1609, doi:10.1038/nm.2535 (2011).

12. Rappuoli, R., De Gregorio, E. & Costantino, P. On the mechanisms of conjugate vaccines. Proc Natl Acad Sci U S A 116, 14-16, doi:10.1073/pnas.1819612116 (2019).

13. Frasch, C. E. Preparation of bacterial polysaccharide-protein conjugates: analytical and manufacturing challenges. Vaccine 27, 6468-6470, doi:10.1016/j.vaccine.2009.06.013 (2009).

14. Pfizer. Pfizer 2015 Annual Report: Manufacturing and Supply Chain. 5 (Pfizer.com, 2015).

15. Feldman, M. F. et al. Engineering N-linked protein glycosylation with diverse O antigen lipopolysaccharide structures in Escherichia coli. Proc Natl Acad Sci U S A 102, 3016-3021, doi:10.1073/pnas.0500044102 (2005).

16. Harding, C. M. & Feldman, M. F. Glycoengineering bioconjugate vaccines, therapeutics, and diagnostics in E. coli. Glycobiology 29, 519-529, doi:10.1093/glycob/cwz031 (2019).

17. Follador, R. et al. The diversity of Klebsiella pneumoniae surface polysaccharides. Microb Genom 2, e000073, doi:10.1099/mgen.0.000073 (2016).

18. Whitfield, C. & Trent, M. S. Biosynthesis and export of bacterial lipopolysaccharides. Annu Rev Biochem 83, 99-128, doi:10.1146/annurev-biochem-060713-035600 (2014).

19. Pan, Y. J. et al. Genetic analysis of capsular polysaccharide synthesis gene clusters in 79 capsular types of Klebsiella spp. Sci Rep 5, 15573, doi:10.1038/srep15573 (2015).

20. Choi, M. et al. The Diversity of Lipopolysaccharide (0) and Capsular Polysaccharide (K) Antigens of Invasive Klebsiella pneumoniae in a Multi-Country Collection. Front Microbiol 11, 1249, doi:10.3389/fmicb.2020.01249 (2020).

21. Clarke, B. R. et al. Molecular basis for the structural diversity in serogroup O2-antigen polysaccharides in Klebsiella pneumoniae. J Biol Chem 293, 4666-4679, doi:10.1074/jbc.RA117.000646 (2018).

22. Clarke, B. R. et al. Role of Rfe and RfbF in the initiation of biosynthesis of D-galactan I, the lipopolysaccharide O antigen from Klebsiella pneumoniae serotype O1. J Bacteriol 177, 5411-5418, doi:10.1128/jb.177.19.5411-5418.1995 (1995).

23. Wick, R. R., Heinz, E., Holt, K. E. & Wyres, K. L. Kaptive Web: User-Friendly Capsule and Lipopolysaccharide Serotype Prediction for Klebsiella Genomes. J Clin Microbiol 56, doi:10.1128/JCM.00197-18 (2018).

24. Clarke, B. R. & Whitfield, C. Molecular cloning of the rib region of Klebsiella pneumoniae serotype O1:K20: the rib gene cluster is responsible for synthesis of the D-galactan I O polysaccharide. J Bacteriol 174, 4614-4621, doi:10.1128/jb.174.14.4614-4621.1992 (1992).

25. Guan, S., Clarke, A. J. & Whitfield, C. Functional analysis of the galactosyltransferases required for biosynthesis of D-galactan I, a component of the lipopolysaccharide O1 antigen of Klebsiella pneumoniae. J Bacteriol 183, 3318-3327, doi:10.1128/JB.183.11.3318-3327.2001 (2001).

26. Szijarto, V. et al. Both clades of the epidemic KPC-producing Klebsiella pneumoniae clone ST258 share a modified galactan O-antigen type. Int J Med Microbiol 306, 89-98, doi:10.1016/j.ijmm 2015.12.002 (2016).

27. Hsieh, P. F. et al. D-galactan II is an immunodominant antigen in O1 lipopolysaccharide and affects virulence in Klebsiella pneumoniae: implication in vaccine design. Front Microbiol 5, 608, doi:10.3389/fmicb.2014.00608 (2014).

28. Kelly, S. D. et al. Klebsiella pneumoniae O1 and O2ac antigens provide prototypes for an unusual strategy for polysaccharide antigen diversification. J Biol Chem 294, 10863-10876, doi:10.1074/jbc.RA119.008969 (2019).

29. Stojkovic, K. et al. Identification of d-Galactan-III As Part of the Lipopolysaccharide of Klebsiella pneumoniae Serotype O1. Front Microbiol 8, 684, doi:10.3389/fmicb.2017.00684 (2017).

30. Greenfield, L. K. et al. Biosynthesis of the polymannose lipopolysaccharide O-antigens from Escherichia coli serotypes O8 and O9a requires a unique combination of single- and multiple-active site mannosyltransferases. J Biol Chem 287, 35078-35091, doi:10.1074/jbc.M112.401000 (2012).

31. Vinogradov, E. et al. Structures of lipopolysaccharides from Klebsiella pneumoniae. Eluicidation of the structure of the linkage region between core and polysaccharide O chain and identification of the residues at the non-reducing termini of the O chains. J Biol Chem 277, 25070-25081, doi:10.1074/jbc.M202683200 (2002).

32. Guachalla, L. M. et al. Discovery of monoclonal antibodies cross-reactive to novel subserotypes of K. pneumoniae O3. Sci Rep 7, 6635, doi:10.1038/s41598-017-06682-2 (2017).

33. Kubler-Kielb, J., Whitfield, C., Katzenellenbogen, E. & Vinogradov, E. Identification of the methyl phosphate substituent at the non-reducing terminal mannose residue of the O-specific polysaccharides of Klebsiella pneumoniae O3, Hafnia alvei PCM 1223 and Escherichia coli O9/O9a LPS. Carbohydr Res 347, 186-188, doi:10.1016/j.carres.2011.11.019 (2012).

34. Artyszuk, D. et al. The Impact of Insertion Sequences on O-Serotype Phenotype and Its O-Locus-Based Prediction in Klebsiella pneumoniae O2 and O1. Int J Mol Sci 21, doi:10.3390/ijms21186572 (2020).

35. Wyres, K. L. et al. Genomic surveillance for hypervirulence and multi-drug resistance in invasive Klebsiella pneumoniae from South and Southeast Asia. Genome Med 12, 11, doi:10.1186/513073-019-0706-y (2020).

36. Feldman, M. F. et al. A promising bioconjugate vaccine against hypervirulent Klebsiella pneumoniae. Proc Natl Acad Sci U S A 116, 18655-18663, doi:10.1073/pnas.1907833116 (2019).

37. Faridmoayer, A., Fentabil, M. A., Mills, D. C., Klassen, J. S. & Feldman, M. F. Functional characterization of bacterial oligosaccharyltransferases involved in O-linked protein glycosylation. J Bacteriol 189, 8088-8098, doi:10.1128/JB.01318-07 (2007).

38. McCallum, K. L., Schoenhals, G., Laakso, D., Clarke, B. & Whitfield, C. A high-molecular-weight fraction of smooth lipopolysaccharide in Klebsiella serotype O1:K20 contains a unique O-antigen epitope and determines resistance to nonspecific serum killing. Infect Immun 57, 3816-3822, doi:10.1128/IAI.57.12.3816-3822.1989 (1989).

39. Saeki, A. et al. Isolation of rfb gene clusters directing the synthesis of O polysaccharides consisting of mannose homopolymers and serological analysis of lipopolysaccharides. Microbiol Immunol 37, 601-606, doi:10.1111/j.1348-0421.1993.tb01682.x (1993).

40. Hultgren, S. J., Normark, S. & Abraham, S. N. Chaperone-assisted assembly and molecular architecture of adhesive pili. Annu Rev Microbiol 45, 383-415, doi:10.1146/annurev.mi.45.100191.002123 (1991).

41. Langstraat, J., Bohse, M. & Clegg, S. Type 3 fimbrial shaft (MrkA) of Klebsiella pneumoniae, but not the fimbrial adhesio (MrkD), facilitates biofilm formation. Infect Immun 69, 5805-5812, doi:10.1128/iai.69.9.5805-5812.2001 (2001).

42. Wang, Q. et al. Target-Agnostic Identification of Functional Monoclonal Antibodies Against Klebsiella pneumoniae Multimeric MrkA Fimbrial Subunit. J Infect Dis 213, 1800-1808, doi:10.1093/infdis/jiw021 (2016).

43. Walczak, M. J., Puorger, C., Glockshuber, R. & Wider, G. Intramolecular donor strand complementation in the E. coli type 1 pilus subunit FimA explains the existence of FimA monomers as off-pathway products of pilus assembly that inhibit host cell apoptosis. J Mol Biol 426, 542-549, doi:10.1016/j jmb.2013.10.029 (2014).

What is claimed is:

1. A bioconjugate comprising a K. pneumoniae O-antigen covalently linked to a fusion protein, wherein the fusion protein comprises a ComP protein or a glycosylation tag fragment thereof,
optionally, wherein the O-antigen has not been derivatized by:
i) being subject to oxidation/reduction procedures;
ii) activated with 1-Cyano-4-Dimethylaminopyridine Tetrafluoroborate (CDAP);
iii) the addition of primary amines; and/or
iv) the addition of diamine spacer molecules, further optionally, wherein the O-antigen is underivatized;
optionally, wherein the O-antigen is a native O-antigen; and/or
optionally, wherein the bioconjugate is immunogenic.

2. The bioconjugate of claim 1, wherein:
the K. pneumoniae O-antigen is selected from the group consisting of O1, O2, O3, O4, O5, O7, O8 and O12;
the K. pneumoniae O-antigen is selected from the group consisting of O1v1, O1v2, O2v1, O2v2, O3, O3a, and O3b; and/or
the K. pneumoniae O-antigen is selected from the group consisting of O1afg, O2afg, O2aeh, and O2ac.

3. The bioconjugate of claim 1, wherein the fusion protein comprises a ComP protein or a glycosylation tag fragment thereof attached to a heterologous carrier protein;
optionally, wherein the ComP protein or a glycosylation tag fragment thereof is attached to the heterologous carrier protein via an amino acid linker;
optionally, wherein the ComP protein or a glycosylation tag fragment thereof is located in the fusion protein C-terminal to the heterologous carrier protein;
optionally, wherein the ComP protein or a glycosylation tag fragment thereof is located in the fusion protein N-terminal to the heterologous carrier protein; and/or
optionally, wherein the fusion protein comprises a signal peptide.

4. The bioconjugate of claim 3, wherein the fusion protein comprises a Pseudomonas aeruginosa exotoxin A (EPA) carrier protein, a CRM197 carrier protein, a tetanus toxin C fragment carrier protein, or a K. pneumoniae MrkA carrier protein;
optionally, wherein the MrkA carrier protein comprises a modified MrkA variant that is self-complemented by translationally fusing a hexaglycine linker and a duplicated MrkA N-terminal donor strand to the C-terminus of the MrkA protein;
optionally, wherein the MrkA carrier protein comprises a native MrkA signal peptide or comprises a DsbA protein signal peptide in place of the MrkA native signal peptide; and/or optionally, wherein the MrkA carrier protein comprises a glycine-glycine-glycine-serine linker linking it to ComP protein or a glycosylation tag fragment thereof.

5. The bioconjugate of claim 1, wherein the glycosylation tag fragment of ComP comprises or consists of an amino acid sequence selected from the group consisting of: SEQ ID NO: 32 [C1]; SEQ ID NO: 33 [D1]; SEQ ID NO: 34 [E1]; SEQ ID NO: 41 [E2]; SEQ ID NO: 42 [F2];
SEQ ID NO: 43 [G2]; SEQ ID NO: 44 [H2]; SEQ ID NO: 45 [A3]; SEQ ID NO: 46 [B3]; SEQ ID NO: 47 [C3]; SEQ ID NO: 55 [D4]; SEQ ID NO: 56 [E4]; SEQ ID NO: 57 [F4]; SEQ ID NO:
58 [G4]; SEQ ID NO: 59 [A5]; SEQ ID NO: 60 [B5]; SEQ ID NO: 61 [D5]; SEQ ID NO: 62 [E5]; SEQ ID NO: 63 [F5]; SEQ ID NO: 72 [H6]; SEQ ID NO: 73 [B7]; SEQ ID NO: 74 [C7];
SEQ ID NO: 75 [D7]; SEQ ID NO: 76 [E7]; SEQ ID NO: 77 [F7]; SEQ ID NO: 78 [A8]; SEQ ID NO: 79 [B8]; SEQ ID NO: 92 [A10]; SEQ ID NO: 93 [B10]; SEQ ID NO: 94 [C10]; SEQ ID NO: 95 [D10]; SEQ ID NO: 96 [F10]; SEQ ID NO: 97 [G10]; SEQ ID NO: 98 [H10]; SEQ ID NO: 99 [A11]; SEQ ID NO: 100 [B11]; and SEQ ID NO: 101 [C11];
optionally, wherein the ComP glycosylation tag does not comprise a methionine residue in a position corresponding to the conserved methionine residue at position 104 of SEQ ID NO: [2](ComP110264: ENV58402.1); and/or
optionally, wherein the amino acid sequence of the ComP glycosylation tag does not extend in the C-terminus direction beyond the amino acid residue corresponding to position 103 of SEQ ID NO: [2](ComP110264: ENV58402.1).

6. The bioconjugate of claim 5, wherein the glycosylation tag fragment of ComP comprises or consists of SEQ ID NO: 32 [C1].

7. The bioconjugate of claim 1, wherein the fusion protein comprises:
SEQ ID NO: 122 (DsbASP-ComP₁₁₀₂₆₄C1_fragment-GGGS-MrkA-GGGGGG-donor_strand);
SEQ ID NO: 124 (DsbASP-MrkA-GGGGGG-donor_strand-GGGS-ComP₁₁₀₂₆₄C1_fragment);
SEQ ID NO: 121 (DsbASP-ComP₁₁₀₂₆₄C1_fragment-GGGS-MrkA-GGGGGG-donor_strand-His); or
SEQ ID NO: 123 (DsbASP-MrkA-GGGGGG-donor_strand-GGGS-ComP₁₁₀₂₆₄C1_fragment-His).

8. The bioconjugate of claim 1, wherein the bioconjugate is a conjugate vaccine.

9. The bioconjugate of claim 1, for use as a conjugate vaccine.

10. The bioconjugate of claim 1, wherein the bioconjugate is produced in vivo; optionally in a bacterial cell.

11. A conjugate vaccine composition comprising the bioconjugate of claim 1.

12. The conjugate vaccine composition of claim 11, wherein the conjugate vaccine composition is a multivalent vaccine comprising at least two, three, four, five, six, or seven of the bioconjugates, each comprising a different K. pneumoniae O-antigen.

13. The conjugate vaccine composition of claim 12, wherein the conjugate vaccine is a multivalent vaccine comprising seven of the bioconjugates each comprising a different K. pneumoniae O-antigen.

14. The conjugate vaccine composition of claim 13, comprising:
(i) a bioconjugate comprising an O1v1 antigen;
(ii) a bioconjugate comprising an O1v2 antigen;
(iii) a bioconjugate comprising an O2v1 antigen;
(iv) a bioconjugate comprising an O2v2 antigen;
(v) a bioconjugate comprising an O3 antigen;
(vi) a bioconjugate comprising an O3 b antigen; and
(vii) a bioconjugate comprising an O5 antigen.

15. The conjugate vaccine composition of claim 11, further comprising an adjuvant.

16. A fusion protein comprising ComP or a glycosylation tag fragment thereof and a Pseudomonas aeruginosa exotoxin A (EPA) carrier protein, a CRM₁₉₇ carrier protein, a tetanus toxin C fragment carrier protein, or a K. pneumoniae MrkA carrier protein;
optionally, wherein the MrkA carrier protein comprises a modified MrkA variant that is self-complemented by translationally fusing a hexaglycine linker and a duplicated MrkA N-terminal donor strand to the C-terminus of the MrkA protein;
optionally, wherein the MrkA carrier protein comprises a native MrkA signal peptide or comprises a DsbA protein signal peptide in place of the MrkA native signal peptide; and/or
optionally, wherein the MrkA carrier protein comprises a glycine-glycine-glycine-serine linker linking it to ComP protein or a glycosylation tag fragment thereof.

17. The fusion protein of claim 16, wherein the fusion protein is covalently linked to a K. pneumoniae O-antigen;
optionally, wherein the O-antigen has not been derivatized by:
i) being subject to oxidation/reduction procedures;
ii) activated with 1-Cyano-4-Dimethylaminopyridine Tetrafluoroborate (CDAP);
iii) the addition of primary amines; and/or
iv) the addition of diamine spacer molecules, further optionally, wherein the O-antigen is underivatized;
optionally, wherein the O-antigen is a native O-antigen; and/or
optionally, wherein the bioconjugate is immunogenic.

18. The fusion protein of claim 17, wherein:
the K. pneumoniae O-antigen is selected from the group consisting of O1, O2, O3, O4, O5, O7, O8 and O12;
the K. pneumoniae O-antigen is selected from the group consisting of O1v1, O1v2, O2v1, O2v2, O3, O3a, and O3b; and/or
the K. pneumoniae O-antigen is selected from the group consisting of O1afg, O2afg, O2aeh, and O2ac.

19. A method of producing a bioconjugate, the method comprising covalently linking a K. pneumoniae O-antigen to a fusion protein with a PglS oligosaccharyltransferase (OTase),
wherein the fusion protein comprises a ComP protein or a glycosylation tag fragment thereof;
optionally, wherein the ComP protein or glycosylation tag fragment thereof is linked to a heterologous carrier protein.

20. A method of inducing a host immune response against K. pneumoniae, the method comprising administering to a subject in need of the immune response an effective amount of the conjugate vaccine composition of any one of claims 11 to 15;
optionally, wherein the subject is a human.

21. The method of claim 20, wherein the immune response is an antibody response.

22. The method of claim 20, wherein the immune response is selected from the group consisting of an innate response, an adaptive response, a humoral response, an antibody response, cell mediated response, a B cell response, a T cell response, cytokine upregulation or downregulation, immune system cross-talk, and a combination of two or more of said immune responses.

23. The method of claim 22, wherein the immune response is selected from the group consisting of an innate response, a humoral response, an antibody response, a T cell response, and a combination of two or more of said immune responses.

24. A method of preventing or treating a K. pneumoniae infection in a subject comprising administering to a subject in need thereof the bioconjugate of any one of claims 1 to 10;
optionally, wherein the subject is a human.

25. Use of the bioconjugate of any one of claims 1 to 10, the conjugate vaccine of any one of claims 11 to 15, or the fusion protein of any one of claims 16 to 18 to induce a host immune response against K. pneumoniae, prevent a K. pneumoniae infection, and/or treat a K. pneumoniae infection.

26. A method of producing a conjugate vaccine against K. pneumoniae infection, the method comprising:
(a) isolating the bioconjugate of any one of claims 1 to 10; and
(b) combining the isolated bioconjugate with an adjuvant.


SEQUENCE LISTING (124 sequences)

SEQ ID NO: 1 - protein, 147 aa, Acinetobacter sp.
MNAQKGFTLIELMIVIAIIGILAAIAIPAYTDYTVRARVSEGLTAASSMKTTVSENILNAGALVAGTPSTAGSSCVGVQEISASNATTNVATATCGASSAGQIIVTMDTTKAKGANITLTPTYASGAVTWKCTTTSDKKYVPSECRG

SEQ ID NO: 2 - protein, 145 aa, Acinetobacter soli
MNAQKGFTLIELMIVIAIIGILAAIAIPAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGT

SEQ ID NO: 3 - protein, 146 aa, Acinetobacter soli
MNAQKGFTLIELMIVIAIIGILAAIAIPAYTDYTVRARVSEGLTTASAMKATVSENILSAGQIVTGTPSTANSSCVGVQEINASSSTSNVATATCSGLGVITVTMDSTKAKGVNLTLTPTYTTSNAVTWKCTTTSDKKYVPSECRN

SEQ ID NO: 4 - protein, 142 aa, Acinetobacter radioresistens
MNTQKGFTLIELMIVIAIIGILAAIAIPAYTDYTVRARVTEAVSTASSMKATVSENIMNAGGTQIPTSGNCVGVQTIAASNATKNVATATCTDSTGVIVVTTTPAAKSVPLTLTPTYTGGNVKWACSTTANFKNYVPSECRS

SEQ ID NO: 5 - protein, 140 aa, Acinetobacter puyangensis
MNAQKGFTLIELMIVIAIIGILAAIAIPAYTDYTVRARVTEALTTASAMKATVSENIMSAGGTTIASSACNGVISASATTNVASSACSGSGVISVTTTAAAKGIVLTLTPKYTGGNVAWQCTTTSGDAQKYVPSECRTTS

SEQ ID NO: 6 - protein, 150 aa, Acinetobacter sp.
MNTQKGFTLIELMIVIAIIGILAAIAIPAYTDYTVRAKVTEAISTASAMKATVSENLMSAGGTSIVSTNANCAGVETIGASNKTKNVESAACTAATGVILVTTTAEAKSVPLTLKPTYTGSNVQWKCGTTAAAFKYVPSECRNDSSGTGF

SEQ ID NO: 7 - protein, 119 aa, Acinetobacter sp.
AYTDYTVRARVSEGLTAASSMKTTVSENILNAGALVAGTPSTAGSSCVGVQEISASNATTNVATATCGASSAGQIIVTMDTTKAKGANITLTPTYASGAVTWKCTTTSDKKYVPSECRG

SEQ ID NO: 8 - protein, 117 aa, Acinetobacter soli
AYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGT

SEQ ID NO: 9 - protein, 118 aa, Acinetobacter soli
AYTDYTVRARVSEGLTTASAMKATVSENILSAGQIVTGTPSTANSSCVGVQEINASSSTSNVATATCSGLGVITVTMDSTKAKGVNLTLTPTYTTSNAVTWKCTTTSDKKYVPSECRN

SEQ ID NO: 10 - protein, 114 aa, Acinetobacter radioresistens
AYTDYTVRARVTEAVSTASSMKATVSENIMNAGGTQIPTSGNCVGVQTIAASNATKNVATATCTDSTGVIVVTTTPAAKSVPLTLTPTYTGGNVKWACSTTANFKNYVPSECRS

SEQ ID NO: 11 - protein, 112 aa, Acinetobacter puyangensis
AYTDYTVRARVTEALTTASAMKATVSENIMSAGGTTIASSACNGVISASATTNVASSACSGSGVISVTTTAAAKGIVLTLTPKYTGGNVAWQCTTTSGDAQKYVPSECRTTS

SEQ ID NO: 12 - protein, 122 aa, Acinetobacter sp.
AYTDYTVRAKVTEAISTASAMKATVSENLMSAGGTSIVSTNANCAGVETIGASNKTKNVESAACTAATGVILVTTTAEAKSVPLTLKPTYTGSNVQWKCGTTAAAFKYVPSECRNDSSGTGF

SEQ ID NO: 13 - protein, 21 aa, Unknown
  Description of Unknown ComP sequence
CVGVQEISASNATTNVATATC

SEQ ID NO: 14 - protein, 23 aa, Unknown
  Description of Unknown ComP sequence
CTGVTQIASGASAATTNVASAQC

SEQ ID NO: 15 - protein, 21 aa, Unknown
  Description of Unknown ComP sequence
CVGVQEINASSSTSNVATATC

SEQ ID NO: 16 - protein, 21 aa, Unknown
  Description of Unknown ComP sequence
CAGVETIGASNKTKNVESAAC

SEQ ID NO: 17 - protein, 21 aa, Unknown
  Description of Unknown ComP sequence
CVGVQTIAASNATKNVATATC

SEQ ID NO: 18 - protein, 335 aa, Artificial Sequence
  Description of Artificial Sequence Synthetic polypeptide
MKKIWLALAGLVLAFSASAAQYEDGKQYTTLEKPVAGAPQVLEFFSFFCPHCYQFEEVLHISDNVKKKLPEGVKMTKYHVNFMGGDLGKDLTQAWAVAMALGVEDKVTVPLFEGVQKTQTIRSASDIRDVFINAGIKGEEYDAAWNSFVVKSLVAQQEKAAADVQLRGVPAMFVNGKYQLNPQGMDTSNMDVFVQQYADTVKYLSEKKGGGSAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGTHHHHHH

SEQ ID NO: 19 - protein, 519 aa, Artificial Sequence
  Description of Artificial Sequence Synthetic polypeptide
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNAAAAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGTHHHHHH

SEQ ID NO: 20 - protein, 758 aa, Artificial Sequence
  Description of Artificial Sequence Synthetic polypeptide
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLTILGWPLAERTVVIPSAIPTDPRNVGGDLDPSSIPDKEQAISALPDYASQPGKPPREDLKGGGSAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGTHHHHHH

SEQ ID NO: 21 - protein, 5 aa, Unknown
  Description of Unknown N-glycosylation bacterial sequence
  MISC_FEATURE (2)..(2): Any amino acid except Pro
DXNST

SEQ ID NO: 22 - protein, 14 aa, Unknown
  Description of Unknown Glycopeptide sequence
ISASNATTNVATAT

SEQ ID NO: 23 - protein, 4 aa, Artificial Sequence
  Description of Artificial Sequence Synthetic peptide
GGGS

SEQ ID NO: 24 - protein, 3 aa, Artificial Sequence
  Description of Artificial Sequence Synthetic peptide
AAA

SEQ ID NO: 25 - protein, 5 aa, Artificial Sequence
  Description of Artificial Sequence Synthetic peptide
PAPAP

SEQ ID NO: 26 - protein, 21 aa, Acinetobacter baylyi
CVGVQEISASNATTNVATATC

SEQ ID NO: 27 - protein, 44 aa, Artificial Sequence
  Description of Artificial Sequence Synthetic polypeptide
  MISC_FEATURE (1)..(1): V, A, or no amino acid
  MISC_FEATURE (2)..(2): A, G, T, or no amino acid
  MISC_FEATURE (5)..(5): P, S, or Q
  MISC_FEATURE (6)..(6): S, M, or I
  MISC_FEATURE (7)..(7): T, P, or V
  MISC_FEATURE (8)..(8): A, S, or T
  MISC_FEATURE (9)..(9): G, N, S, or T
  MISC_FEATURE (10)..(10): N or no amino acid
  MISC_FEATURE (11)..(11): S, G, or A
  MISC_FEATURE (12)..(12): S or N
  MISC_FEATURE (14)..(14): V, T, or A
  MISC_FEATURE (17)..(17): Q, T, or E
  MISC_FEATURE (18)..(18): E, Q, or T
  MISC_FEATURE (20)..(20): S, N, A, or G
  MISC_FEATURE (21)..(21): S or no amino acid
  MISC_FEATURE (22)..(22): G or no amino acid
  MISC_FEATURE (25)..(25): N, S, or A
  MISC_FEATURE (26)..(26): A, S, or K
  MISC_FEATURE (28)..(28): T, S, or K
  MISC_FEATURE (31)..(31): A or E
  MISC_FEATURE (32)..(32): T or S
  MISC_FEATURE (34)..(34): T, Q, or A
  MISC_FEATURE (36)..(36): G, S, or T
  MISC_FEATURE (37)..(37): A, G, or D
  MISC_FEATURE (38)..(38): S, L, or A
  MISC_FEATURE (39)..(39): S, G, D, or T
  MISC_FEATURE (40)..(40): A, V, or G
  MISC_FEATURE (41)..(41): G, I, or V
  MISC_FEATURE (42)..(42): Q, T, or I
  MISC_FEATURE (43)..(43): I, V, T, or L
  MISC_FEATURE (44)..(44): I, T, or V
XXGTXXXXXNXXCXGVXXIXSGASXXTXNVXXAXCXXXXXXXXX

SEQ ID NO: 28 - protein, 23 aa, Artificial Sequence
  Description of Artificial Sequence Synthetic peptide
  MISC_FEATURE (2)..(2): V,T, or A
  MISC_FEATURE (5)..(5): Q, T, or E
  MISC_FEATURE (6)..(6): E, Q, or T
  MISC_FEATURE (8)..(8): S, N, A, or G
  MISC_FEATURE (9)..(9): S or no amino acid
  MISC_FEATURE (10)..(10): G or no amino acid
  MISC_FEATURE (13)..(13): N, S, or A
  MISC_FEATURE (14)..(14): A, S, or K
  MISC_FEATURE (16)..(16): T, S, or K
  MISC_FEATURE (19)..(19): A or E
  MISC_FEATURE (20)..(20): T or S
  MISC_FEATURE (22)..(22): T, Q, or A
CXGVXXIXSGASXXTXNVXXAXC

SEQ ID NO: 29 - protein, 117 aa, Acinetobacter soli; identical to SEQ ID NO: 8

SEQ ID NO: 30 - protein, 25 aa, Acinetobacter soli
SSGNCTGVTQIASGASAATTNVASA

SEQ ID NO: 31 - protein, 25 aa, Acinetobacter soli
SGNCTGVTQIASGASAATTNVASAQ

SEQ ID NO: 32 - protein, 25 aa, Acinetobacter soli
GNCTGVTQIASGASAATTNVASAQC

SEQ ID NO: 33 - protein, 25 aa, Acinetobacter soli
NCTGVTQIASGASAATTNVASAQCS

SEQ ID NO: 34 - protein, 25 aa, Acinetobacter soli
CTGVTQIASGASAATTNVASAQCSD

SEQ ID NO: 35 - protein, 25 aa, Acinetobacter soli
TGVTQIASGASAATTNVASAQCSDS

SEQ ID NO: 36 - protein, 25 aa, Acinetobacter soli
GVTQIASGASAATTNVASAQCSDSD

SEQ ID NO: 37 - protein, 25 aa, Acinetobacter soli
VTQIASGASAATTNVASAQCSDSDG

SEQ ID NO: 38 - protein, 25 aa, Acinetobacter soli
TQIASGASAATTNVASAQCSDSDGV

SEQ ID NO: 39 - protein, 30 aa, Acinetobacter soli
GTSMPSSGNCTGVTQIASGASAATTNVASA

SEQ ID NO: 40 - protein, 30 aa, Acinetobacter soli
TSMPSSGNCTGVTQIASGASAATTNVASAQ

SEQ ID NO: 41 - protein, 30 aa, Acinetobacter soli
SMPSSGNCTGVTQIASGASAATTNVASAQC

SEQ ID NO: 42 - protein, 30 aa, Acinetobacter soli
MPSSGNCTGVTQIASGASAATTNVASAQCS

SEQ ID NO: 43 - protein, 30 aa, Acinetobacter soli
PSSGNCTGVTQIASGASAATTNVASAQCSD

SEQ ID NO: 44 - protein, 30 aa, Acinetobacter soli
SSGNCTGVTQIASGASAATTNVASAQCSDS

SEQ ID NO: 45 - protein, 30 aa, Acinetobacter soli
SGNCTGVTQIASGASAATTNVASAQCSDSD

SEQ ID NO: 46 - protein, 30 aa, Acinetobacter soli
GNCTGVTQIASGASAATTNVASAQCSDSDG

SEQ ID NO: 47 - protein, 30 aa, Acinetobacter soli
NCTGVTQIASGASAATTNVASAQCSDSDGV

SEQ ID NO: 48 - protein, 30 aa, Acinetobacter soli
TGVTQIASGASAATTNVASAQCSDSDGVIT

SEQ ID NO: 49 - protein, 30 aa, Acinetobacter soli
GVTQIASGASAATTNVASAQCSDSDGVITV

SEQ ID NO: 50 - protein, 30 aa, Acinetobacter soli
VTQIASGASAATTNVASAQCSDSDGVITVT

SEQ ID NO: 51 - protein, 30 aa, Acinetobacter soli
TQIASGASAATTNVASAQCSDSDGVITVTM

SEQ ID NO: 52 - protein, 30 aa, Acinetobacter soli
QIASGASAATTNVASAQCSDSDGVITVTMT

SEQ ID NO: 53 - protein, 35 aa, Acinetobacter soli
IMNAGGTSMPSSGNCTGVTQIASGASAATTNVASA

SEQ ID NO: 54 - protein, 35 aa, Acinetobacter soli
MNAGGTSMPSSGNCTGVTQIASGASAATTNVASAQ

SEQ ID NO: 55 - protein, 35 aa, Acinetobacter soli
NAGGTSMPSSGNCTGVTQIASGASAATTNVASAQC

SEQ ID NO: 56 - protein, 35 aa, Acinetobacter soli
AGGTSMPSSGNCTGVTQIASGASAATTNVASAQCS

SEQ ID NO: 57 - protein, 35 aa, Acinetobacter soli
GGTSMPSSGNCTGVTQIASGASAATTNVASAQCSD

SEQ ID NO: 58 - protein, 35 aa, Acinetobacter soli
GTSMPSSGNCTGVTQIASGASAATTNVASAQCSDS

SEQ ID NO: 59 - protein, 35 aa, Acinetobacter soli
SMPSSGNCTGVTQIASGASAATTNVASAQCSDSDG

SEQ ID NO: 60 - protein, 35 aa, Acinetobacter soli
MPSSGNCTGVTQIASGASAATTNVASAQCSDSDGV

SEQ ID NO: 61 - protein, 35 aa, Acinetobacter soli
SSGNCTGVTQIASGASAATTNVASAQCSDSDGVIT

SEQ ID NO: 62 - protein, 35 aa, Acinetobacter soli
SGNCTGVTQIASGASAATTNVASAQCSDSDGVITV

SEQ ID NO: 63 - protein, 35 aa, Acinetobacter soli
GNCTGVTQIASGASAATTNVASAQCSDSDGVITVT

SEQ ID NO: 64 - protein, 35 aa, Acinetobacter soli
NCTGVTQIASGASAATTNVASAQCSDSDGVITVTM

SEQ ID NO: 65 - protein, 35 aa, Acinetobacter soli
CTGVTQIASGASAATTNVASAQCSDSDGVITVTMT

SEQ ID NO: 66 - protein, 35 aa, Acinetobacter soli
TGVTQIASGASAATTNVASAQCSDSDGVITVTMTD

SEQ ID NO: 67 - protein, 35 aa, Acinetobacter soli
GVTQIASGASAATTNVASAQCSDSDGVITVTMTDK

SEQ ID NO: 68 - protein, 35 aa, Acinetobacter soli
VTQIASGASAATTNVASAQCSDSDGVITVTMTDKA

SEQ ID NO: 69 - protein, 35 aa, Acinetobacter soli
TQIASGASAATTNVASAQCSDSDGVITVTMTDKAK

SEQ ID NO: 70 - protein, 40 aa, Acinetobacter soli
TVSENIMNAGGTSMPSSGNCTGVTQIASGASAATTNVASA

SEQ ID NO: 71 - protein, 40 aa, Acinetobacter soli
VSENIMNAGGTSMPSSGNCTGVTQIASGASAATTNVASAQ

SEQ ID NO: 72 - protein, 40 aa, Acinetobacter soli
SENIMNAGGTSMPSSGNCTGVTQIASGASAATTNVASAQC

SEQ ID NO: 73 - protein, 40 aa, Acinetobacter soli
NIMNAGGTSMPSSGNCTGVTQIASGASAATTNVASAQCSD

SEQ ID NO: 74 - protein, 40 aa, Acinetobacter soli
IMNAGGTSMPSSGNCTGVTQIASGASAATTNVASAQCSDS

SEQ ID NO: 75 - protein, 40 aa, Acinetobacter soli
MNAGGTSMPSSGNCTGVTQIASGASAATTNVASAQCSDSD

SEQ ID NO: 76 - protein, 40 aa, Acinetobacter soli
NAGGTSMPSSGNCTGVTQIASGASAATTNVASAQCSDSDG

SEQ ID NO: 77 - protein, 40 aa, Acinetobacter soli
AGGTSMPSSGNCTGVTQIASGASAATTNVASAQCSDSDGV

SEQ ID NO: 78 - protein, 40 aa, Acinetobacter soli
TSMPSSGNCTGVTQIASGASAATTNVASAQCSDSDGVITV

SEQ ID NO: 79 - protein, 40 aa, Acinetobacter soli
SMPSSGNCTGVTQIASGASAATTNVASAQCSDSDGVITVT

SEQ ID NO: 80 - protein, 40 aa, Acinetobacter soli
MPSSGNCTGVTQIASGASAATTNVASAQCSDSDGVITVTM

SEQ ID NO: 81 - protein, 40 aa, Acinetobacter soli
PSSGNCTGVTQIASGASAATTNVASAQCSDSDGVITVTMT

SEQ ID NO: 82 - protein, 40 aa, Acinetobacter soli
SSGNCTGVTQIASGASAATTNVASAQCSDSDGVITVTMTD

SEQ ID NO: 83 - protein, 40 aa, Acinetobacter soli
SGNCTGVTQIASGASAATTNVASAQCSDSDGVITVTMTDK

SEQ ID NO: 84 - protein, 40 aa, Acinetobacter soli
GNCTGVTQIASGASAATTNVASAQCSDSDGVITVTMTDKA

SEQ ID NO: 85 - protein, 40 aa, Acinetobacter soli
NCTGVTQIASGASAATTNVASAQCSDSDGVITVTMTDKAK

SEQ ID NO: 86 - protein, 40 aa, Acinetobacter soli
CTGVTQIASGASAATTNVASAQCSDSDGVITVTMTDKAKG

SEQ ID NO: 87 - protein, 40 aa, Acinetobacter soli
TGVTQIASGASAATTNVASAQCSDSDGVITVTMTDKAKGV

SEQ ID NO: 88 - protein, 40 aa, Acinetobacter soli
GVTQIASGASAATTNVASAQCSDSDGVITVTMTDKAKGVS

SEQ ID NO: 89 - protein, 40 aa, Acinetobacter soli
VTQIASGASAATTNVASAQCSDSDGVITVTMTDKAKGVSI

SEQ ID NO: 90 - protein, 40 aa, Acinetobacter soli
TQIASGASAATTNVASAQCSDSDGVITVTMTDKAKGVSIK

SEQ ID NO: 91 - protein, 45 aa, Acinetobacter soli
AMKATVSENIMNAGGTSMPSSGNCTGVTQIASGASAATTNVASAQ

SEQ ID NO: 92 - protein, 45 aa, Acinetobacter soli
MKATVSENIMNAGGTSMPSSGNCTGVTQIASGASAATTNVASAQC

SEQ ID NO: 93 - protein, 45 aa, Acinetobacter soli
KATVSENIMNAGGTSMPSSGNCTGVTQIASGASAATTNVASAQCS

SEQ ID NO: 94 - protein, 45 aa, Acinetobacter soli
ATVSENIMNAGGTSMPSSGNCTGVTQIASGASAATTNVASAQCSD

SEQ ID NO: 95 - protein, 45 aa, Acinetobacter soli
TVSENIMNAGGTSMPSSGNCTGVTQIASGASAATTNVASAQCSDS

SEQ ID NO: 96 - protein, 45 aa, Acinetobacter soli
SENIMNAGGTSMPSSGNCTGVTQIASGASAATTNVASAQCSDSDG

SEQ ID NO: 97 - protein, 45 aa, Acinetobacter soli
ENIMNAGGTSMPSSGNCTGVTQIASGASAATTNVASAQCSDSDGV

SEQ ID NO: 98 - protein, 45 aa, Acinetobacter soli
NIMNAGGTSMPSSGNCTGVTQIASGASAATTNVASAQCSDSDGVI

SEQ ID NO: 99 - protein, 45 aa, Acinetobacter soli
IMNAGGTSMPSSGNCTGVTQIASGASAATTNVASAQCSDSDGVIT

SEQ ID NO: 100 - protein, 45 aa, Acinetobacter soli
MNAGGTSMPSSGNCTGVTQIASGASAATTNVASAQCSDSDGVITV

SEQ ID NO: 101 - protein, 45 aa, Acinetobacter soli
NAGGTSMPSSGNCTGVTQIASGASAATTNVASAQCSDSDGVITVT

SEQ ID NO: 102 - protein, 45 aa, Acinetobacter soli
AGGTSMPSSGNCTGVTQIASGASAATTNVASAQCSDSDGVITVTM

SEQ ID NO: 103 - protein, 45 aa, Acinetobacter soli
GGTSMPSSGNCTGVTQIASGASAATTNVASAQCSDSDGVITVTMT

SEQ ID NO: 104 - protein, 45 aa, Acinetobacter soli
GTSMPSSGNCTGVTQIASGASAATTNVASAQCSDSDGVITVTMTD

SEQ ID NO: 105 - protein, 45 aa, Acinetobacter soli
SMPSSGNCTGVTQIASGASAATTNVASAQCSDSDGVITVTMTDKA

SEQ ID NO: 106 - protein, 45 aa, Acinetobacter soli
MPSSGNCTGVTQIASGASAATTNVASAQCSDSDGVITVTMTDKAK

SEQ ID NO: 107 - protein, 45 aa, Acinetobacter soli
PSSGNCTGVTQIASGASAATTNVASAQCSDSDGVITVTMTDKAKG

SEQ ID NO: 108 - protein, 45 aa, Acinetobacter soli
SSGNCTGVTQIASGASAATTNVASAQCSDSDGVITVTMTDKAKGV

SEQ ID NO: 109 - protein, 45 aa, Acinetobacter soli
SGNCTGVTQIASGASAATTNVASAQCSDSDGVITVTMTDKAKGVS

SEQ ID NO: 110 - protein, 45 aa, Acinetobacter soli
GNCTGVTQIASGASAATTNVASAQCSDSDGVITVTMTDKAKGVSI

SEQ ID NO: 111 - protein, 45 aa, Acinetobacter soli
NCTGVTQIASGASAATTNVASAQCSDSDGVITVTMTDKAKGVSIK

SEQ ID NO: 112 - protein, 45 aa, Acinetobacter soli
CTGVTQIASGASAATTNVASAQCSDSDGVITVTMTDKAKGVSIKL

SEQ ID NO: 113 - protein, 45 aa, Acinetobacter soli
TGVTQIASGASAATTNVASAQCSDSDGVITVTMTDKAKGVSIKLT

SEQ ID NO: 114 - protein, 6 aa, Artificial Sequence
  Description of Artificial Sequence Synthetic 6xHis tag
HHHHHH

SEQ ID NO: 115 - protein, 32 aa, Acinetobacter soli
SSGNCTGVTQIASGASAATTNVASAQCSDSDG

SEQ ID NO: 116 - protein, 5 aa, Artificial Sequence
  Description of Artificial Sequence Synthetic peptide
GGGGS

SEQ ID NO: 117 - protein, 758 aa, Artificial Sequence; identical to SEQ ID NO: 20
  Synthetic Fusion Protein

SEQ ID NO: 118 - protein, 752 aa, Artificial Sequence
  Synthetic Fusion Protein
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLTILGWPLAERTVVIPSAIPTDPRNVGGDLDPSSIPDKEQAISALPDYASQPGKPPREDLKGGGSAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGT

SEQ ID NO: 119 - protein, 352 aa, Artificial Sequence
  Synthetic Fusion Protein
MKKIWLALAGLVLAFSASAADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQGGGGGGADTNVGGGQVNFFGKVTDVSGGGSAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGTHHHHHH

SEQ ID NO: 120 - protein, 346 aa, Artificial Sequence
  Synthetic Fusion Protein
MKKIWLALAGLVLAFSASAADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQGGGGGGADTNVGGGQVNFFGKVTDVSGGGSAYTDYTVRSRVTEGLTTASAMKATVSENIMNAGGTSMPSSGNCTGVTQIASGASAATTNVASAQCSDSDGVITVTMTDKAKGVSIKLTPSFSSTGSVGWKCTTSSDKKYVPSECRGT

SEQ ID NO: 121 - protein, 263 aa, Artificial Sequence
  Synthetic Fusion Protein
MKKIWLALAGLVLAFSASAGNCTGVTQIASGASAATTNVASAQCGGGSADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQGGGGGGADTNVGGGQVNFFGKVTDVSGSGHHHHHH

SEQ ID NO: 122 - protein, 257 aa, Artificial Sequence
  Synthetic Fusion Protein
MKKIWLALAGLVLAFSASAGNCTGVTQIASGASAATTNVASAQCGGGSADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQGGGGGGADTNVGGGQVNFFGKVTDVSGSG

SEQ ID NO: 123 - protein, 260 aa, Artificial Sequence
  Synthetic Fusion Protein
MKKIWLALAGLVLAFSASAADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQGGGGGGADTNVGGGQVNFFGKVTDVSGGGSGNCTGVTQIASGASAATTNVASAQCHHHHHH

SEQ ID NO: 124 - protein, 254 aa, Artificial Sequence
  Synthetic Fusion Protein
MKKIWLALAGLVLAFSASAADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQGGGGGGADTNVGGGQVNFFGKVTDVSGGGSGNCTGVTQIASGASAATTNVASAQC